(19) United States
(12) Patent Application Publication
(10) Pub. No.: US 2023/0000896 A1
(43) Pub. Date: Jan. 5, 2023

(54) ALDH3A2 INHIBITION AND FERROPTOSIS INDUCTION FOR CANCER THERAPY

(71) Applicants: Rushdia Yusuf, Boston, MA (US); Shrikanta Chattopadhyay, Cambridge, MA (US); President and Fellows of Harvard College, Cambridge, MA (US); The Broad Institute, Inc., Cambridge, MA (US); The General Hospital Corporation, Boston, MA (US)

(72) Inventors: David T. Scadden, Cambridge, MA (US); Rushdia Yusuf, Boston, MA (US); Nick Van Gastel, Bertem (BE); Azeem S. Sharda, Medford, MA (US); Borja Saez, Pamplona (ES); Shrikanta Chattopadhyay, Cambridge, MA (US); Vasanthi S. Viswanathan, Arlington, MA (US)

(21) Appl. No.: 17/783,573

(22) PCT Filed: Dec. 9, 2020
(86) PCT No.: PCT/US2020/064136

§ 371 Date: Jun. 8, 2022

Related U.S. Application Data
(60) Provisional application No. 62/980,989, filed on Feb. 24, 2020.
(60) Provisional application No. 62/945,880, filed on Dec. 9, 2019.

(51) Int. Cl. A61K 31/713; A61P 35/00; A61K 31/381; A61K 31/437; A61K 31/496

(52) U.S. Cl. CPC A61K 31/713; A61P 35/00; A61K 31/381; A61K 31/437; A61K 31/496

(57) ABSTRACT

Disclosed herein are methods and compositions for inhibiting Aldh3a2 expression and activity to improve leukemia outcomes. Aldh3a2 depletion results in iron-dependent oxidative cell death of leukemia cells while sparing normal hematopoiesis.

### RELATED APPLICATION

This application claims the benefit of U.S. Provisional Application Ser. No. 62/980,989 filed Feb. 24, 2020 and U.S. Provisional Application Ser. No. 62/945,880, filed on Dec. 9, 2019. The entire teachings of the above applications are incorporated herein by reference in their entirety.

### BACKGROUND OF THE INVENTION

Acute myeloid leukemia (AML) is a highly lethal hematopoietic cancer that annually affects 20,000 people and claims 13,000 lives in the U.S. alone. Great strides have been made in understanding the cytogenetic changes and genetic mutations associated with AML. New therapeutic strategies are being tested, but the survival of AML patients remains poor. Even with full adoption of the currently available mutation-specific therapies, only a minority of AML patients can benefit. There is thus a need for new therapeutic strategies.

Drugs that are currently used to treat AML kill cancer cells efficiently in vitro, except when bone marrow stroma is present. It was therefore hypothesized that an ex vivo system mimicking the tissue microenvironment would be required to define distinctive, genotype-independent vulnerabilities of malignant cells. The metabolism of cancer cells and their normal tissue counterparts is distinct. Within the heterogeneous populations of some cancers, stem-like cells associated with treatment resistance may also have different metabolic dependencies. These are not readily identified by genotypic or transcriptomic analyses and may offer promising and largely unmined therapeutic possibilities.

The main treatment for most types of AML is chemotherapy. This might be followed by a stem cell transplant, high dose chemotherapy or targeted therapy upon relapse.

Chemotherapy treatment of AML is usually divided into 2 phases: ‘induction’ and ‘consolidation’. Induction is short and intensive, typically lasting about a week, and has the goal to clear the blood of leukemia cells and to reduce the number of blasts in the bone marrow to normal. After confirmation of a remission status, consolidation is given in cycles after the patient has recovered from induction and is meant to kill the residual leukemia cells. Conventional AML chemotherapy is based on intensive use of cytarabine or other nucleoside analogs in combination with anthracyclines such as daunorubicin, idarubicin or doxorubicin. Although chemotherapy regimens will induce complete remission in the majority of patients, relapse rates are very high. Relapsed AML is generally lethal with few opportunities for cure. Preventing relapse is therefore a key challenge in the treatment of AML.

Patients who fail the chemotherapy induction typically receive matched sibling or alternative donor hematopoietic cell transplantation, high dose chemotherapy or targeted therapy for patients with defined mutations or CD33-positive AML. Given their specificity, only a minority of AML patients can benefit from these targeted therapies, and the problem of therapy resistance and disease relapse remains.

### SUMMARY OF THE INVENTION

It has been surprisingly found that AML cells (e.g., leukemic stem cells) but not normal blood cells (e.g., granulocyte-monocyte progenitors) are susceptible to Aldh3a2 inhibition and that such inhibition in combination with induction of ferroptosis is lethal to cancer cells without inducing cell death of non-cancerous cells.

Some aspects of the disclosure are related to a method of inducing cell death of cancer cells in a population of cells comprising contacting the population of cells with an effective amount of a first agent inhibiting Aldh3a2 activity or expression and an effective amount of a second agent inducing ferroptosis. In some embodiments, the method also inhibits the growth of the cancer cells. In some embodiments, the method does not induce or does not substantially induce cell death of non-cancer cells. In some embodiments, the uncontacted cancer cells exhibit increased redox stress or excessive production of reactive oxygen species. In some embodiments, the cancer cells are leukemia cells. In some embodiments, the cancer cells are AML cells. In some embodiments, the cancer cells are human cancer cells.

In some embodiments, the first agent is a polypeptide, nucleic acid, or small molecule. In some embodiments, the first agent is a short hairpin RNA (shRNA) inhibiting Aldh3a2 expression. In some embodiments, the second agent is a polypeptide, nucleic acid, or small molecule. In some embodiments, the second agent is a glutathione peroxidase 4 (GPX4) inhibitor. In some embodiments, the GPX4 inhibitor is erastin, RSL3, ML162, or DPI10. In some embodiments, the method is performed in vivo, in vitro, or ex vivo. In some embodiments, the first agent and second agent are contacted with the population of cells simultaneously or sequentially.

Some aspects of the disclosure are related to an anti-cancer composition, comprising a first agent which inhibits Aldh3a2 activity or expression, and a second agent which induces ferroptosis. In some embodiments, the first agent is a short hairpin RNA (shRNA) inhibiting Aldh3a2 expression. In some embodiments, the second agent is a polypeptide, nucleic acid, or small molecule. In some embodiments, the second agent is a glutathione peroxidase 4 (GPX4) inhibitor. In some embodiments, the GPX4 inhibitor is erastin, RSL3, ML162, or DPI10. In certain aspects, the composition is formulated for administration by a mode selected from the group consisting of: topically, by injection, by intravenous injection, by inhalation, continuous release by depot or pump, and a combination thereof.

Some aspects of the disclosure are related to a method of treating cancer in a subject in need thereof, comprising administering to the subject an effective amount of a first agent inhibiting Aldh3a2 activity or expression and a chemotherapeutic regimen. In some embodiments, the chemotherapeutic regimen is an induction chemotherapy treatment regimen to the subject. In some embodiments, the induction chemotherapy comprises administering an antimetabolite agent and an anthracycline agent to the subject. In some embodiments, the cancer is leukemia. In some embodiments, the subject is human.

The practice of the present invention will typically employ, unless otherwise indicated, conventional techniques of cell biology, cell culture, molecular biology, transgenic biology, microbiology, recombinant nucleic acid (e.g., DNA) technology, immunology, and RNA interference (RNAi) which are within the skill of the art. Non-limiting descriptions of certain of these techniques are found in the following publications: Ausubel, F., et al., (eds.), Current Protocols in Molecular Biology, Current Protocols in Immunology, Current Protocols in Protein Science, and Current Protocols in Cell Biology, all John Wiley & Sons, N.Y., edition as of December 2008; Sambrook, Russell, and Sambrook, Molecular Cloning: A Laboratory Manual, 3rd ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988; Freshney, R. I., “Culture of Animal Cells, A Manual of Basic Technique”, 5th ed., John Wiley & Sons, Hoboken, N.J., 2005. Non-limiting information regarding therapeutic agents and human diseases is found in Goodman and Gilman's The Pharmacological Basis of Therapeutics, 11th Ed., McGraw Hill, 2005, Katzung, B. (ed.) Basic and Clinical Pharmacology, McGraw-Hill/Appleton & Lange; 10th ed. (2006) or 11th edition (July 2009). Non-limiting information regarding genes and genetic disorders is found in McKusick, V. A.: Mendelian Inheritance in Man. A Catalog of Human Genes and Genetic Disorders. Baltimore: Johns Hopkins University Press, 1998 (12th edition) or the more recent online database: Online Mendelian Inheritance in Man, OMIM™. McKusick-Nathans Institute of Genetic Medicine, Johns Hopkins University (Baltimore, Md.) and National Center for Biotechnology Information, National Library of Medicine (Bethesda, Md.), as of May 1, 2010, ncbi.nlm.nih.gov/omim/ and in Online Mendelian Inheritance in Animals (OMIA), a database of genes, inherited disorders and traits in animal species (other than human and mouse), at omia.angis.org.au/contact.shtml. All patents, patent applications, and other publications (e.g., scientific articles, books, websites, and databases) mentioned herein are incorporated by reference in their entirety. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.

The above discussed, and many other features and attendant advantages of the present inventions will become better understood by reference to the following detailed description of the invention in conjunction with the attached drawings.

### BRIEF DESCRIPTION OF THE DRAWINGS

FIGS. 1A-1D show that loss-of-function screen reveals metabolic vulnerabilities in MLL-AF9 driven AML. FIG. 1A shows a heat-map showing forty-five metabolic genes over-expressed in L-GMPs as compared to N-GMPs in the retrovirus based MLL-AF9 leukemia mode123. FIG. 1B shows the experimental workflow for the genetic depletion screen. FIG. 1C shows the distribution of the number of shRNA-modified L-GMPs per well relative to the number of L-GMPs per control well on the respective plate. Any gene for which three or more shRNAs decreased the number of L-GMPs below 1.5 standard deviation of the mean of L-GMPs in control wells, in technical duplicate, was counted as a hit as long as no more than one shRNA decreased numbers of N-GMPs below 1.5 standard deviation for the same gene. Aldh3a2 amongst ALL sh-RNAs is shown in red. FIG. 1D shows the number of L-GMPs were decreased below 1.5 SD in three wells carrying independent Aldh3a2 shRNAs in the screen.

FIGS. 2A-2E show that Aldh3a2 is essential for leukemia cells in vitro and in vivo. FIG. 2A shows relative Aldh3a2 expression in L-GMPs versus N-GMPs23. FIG. 2B shows validation of Aldh3a2 knockdown by two independent shRNAs (Aldh3a2-sh-1 and Aldh3a2-sh-2 from the screen) by Q-PCR. FIG. 2C shows Aldh3a2 knockdown with two independent shRNAs significantly decreases the number of L-GMPs in methylcellulose compared to L-GMPs infected with control shRNA. FIG. 2D shows L-GMPs were infected with shRNAs Aldh3a2-sh-1, Aldh3a2-sh-2 or control shRNA and injected into sublethally irradiated C57BL6/J mice for disease development. Kaplan-Meier survival curve of animals that developed leukemia is shown. FIG. 2E shows sublethally irradiated C57BL/6J mice were injected with Aldh3a2fl/fl:Mx1-cre+ (Aldh3a2-mut) and Aldh3a2fl/fl:Mx1-cre− (Aldh3a2-control) leukemia cells from primary leukemic mice. Forty eight hours after injection mice were injected with 3 doses of Poly(I):Poly(C) on alternate days and leukemia development was monitored. Kaplan-Meier survival curve of animals that developed leukemia is shown. Data are representative of at least 2 independent experiments; n=5 mice per genotype per experiment. Data are represented as mean+/−SD. nsP>0.05; *P<0.05; **P<0.01; ***P<0.001.

FIGS. 3A-3G illustrate that Aldh3a2 is dispensable for normal hematopoiesis. FIG. 3A shows Aldh3a2 knockdown with Aldh3a2-sh-1 shows no difference in the number of N-GMPs in methylcellulose compared to N-GMPs infected with control shRNA (n=3 wells per group). FIG. 3B depicts Aldh3a2−/− mice (KO) show one half of total aldehyde dehydrogenase enzyme activity in whole bone marrow compared to Aldh3a2+/+ mice (WT). FIGS. 3C-3E show BM analysis showing frequency of (3C) LKS CD48− CD150+ HSCs; (3D) committed myeloid (Granulocyte Monocyte or Macrophage Progenitors (GMPs), Common Myeloid Progenitors (CMP), Megakaryocyte Erythrocyte Progenitors (MEP) progenitors and (3E) B cells (B220) and myeloid cells (Macl) in Aldh3a2-WT and Aldh3a2-KO mice. FIGS. 3F-3G show relative peripheral blood (PB) reconstitution (3F) and contribution to B cells (B220), myeloid cells (Mac 1) and T cells (CD3) (3G), 20 weeks after transplantation, of recipient B6.SJL (CD45.1) mice transfused with whole BM cells from Aldh3a2-WT or KO mice (CD45.2+) competed with equal numbers of wild-type CD45.1 whole BM cells (n=10 recipients per group). Data are representative of at least 2 independent experiments; n=3 mice per genotype per experiment. Data are represented as mean+/−SD. nsP>0.05; *P<0.05; **P<0.01; ***P<0.001.

FIGS. 4A-4F show human leukemia is sensitive to ALDH3A2 depletion. FIG. 4A shows ALDH3A2 expression in pre-treatment AML patients with normal karyotype from the Meltezer database. Data was divided into ALDH3A2 high and ALDH3A2 low patients around the median of the distribution of ALDH3A2 expression. FIG. 4B shows Overall Survival (OS) in patients with high versus low ALDH3A2 expression. FIG. 4C shows relative ALDH3A2 expression in human AML cell lines. FIG. 4D shows the effective knockdown of ALDH3A2 expression in THP1 cells with two independent shRNAs (ALDH3A2-sh-A and ALDH3A2-sh-B) compared to control shRNA. FIG. 4E shows ALDH3A2 knockdown by two independent shRNAs decreases cell growth in five different AML cell lines as compared to cells infected with control shRNA. FIG. 4F shows ALDH3A2 knockdown by one or two independent shRNAs (depending on number of patient cells available) decreased the growth of three different primary AML samples as compared to cells infected with control shRNA. Data are representative of at least 2 independent experiments; n=3 replicates per cell line per experiment. Data are represented as mean+/−SD. nsP>0.05; *P<0.05; **P<0.01; ***P<0.001.

FIGS. 5A-5E illustrate Aldh3a2 depletion alters the redox state of cells. FIGS. 5A-5B show cellular ROS (5A) and lipid peroxidation (5B) levels in Aldh3a2-control and mutant leukemic stem and progenitor cells (LSPCs: Linlow and c-Kit+). FIGS. 5C-5D show cellular ROS (5C) and lipid peroxidation levels (5D) in Aldh3a2-control and mutant LSPCs upon 4HNE and 4HNE plus vitamin E treatment. FIG. 5E shows growth kinetics of Aldh3a2-control and mutant LSPCs treated with 4HNE. Data are representative of at least 2 independent experiments; n=3 replicates per cell line per experiment. Data are represented as mean+/−SD. nsP>0.05; *P<0.05; **P<0.01; ***P<0.001.

FIGS. 6A-6J show Aldh3a2 depletion causes death by ferroptosis. FIGS. 6A-6C demonstrate Aldh3a2 mutant leukemia cells show evidence of oxidative damage to DNA and protein as shown by increased levels of gamma-H2AX (6A) 8-OHDG (6B) and protein carbonylation (6C) in mutant versus control cells. FIGS. 6D-6E show caspase 3 activation (6D) and PARP cleavage (6E) in Aldh3a2-mutant versus control LSPCs. FIG. 6F shows the frequency of live Aldh3a2-control and mutant LSPCs three days after culture and treatment with DMSO and ZVAD reveal no rescue from cell death. FIG. 6G shows assessment of cell cycle in Aldh3a2-control and mutant leukemia cells reveals no difference in profiles. FIG. 6H shows the frequency of live Aldh3a2-control and mutant LSPCs three days after culture and treatment with DMSO and Ferrostatin reveals complete rescue of mutant cells from cell death pointing toward ferroptosis as mechanism of death of these cells. FIG. 6I shows the frequency of live Aldh3a2-control and mutant LSPCs three days after culture and treatment with DMSO and RSL3 reveal significantly increased susceptibility to death from this Gpx4 inhibitor showing susceptibility to ferroptosis. FIG. 6J shows sublethally irradiated C57BL/6J mice were injected with Aldh3a2-control and mutant leukemia cells, from primary leukemic mice, infected with lentivirus expressing Gpx4 or scrambled sRNA. Forty eight hours after injection mice were injected with 3 doses of Poly(I):Poly(C) on alternate days and leukemia development was monitored. Kaplan-Meier survival curve of animals that developed leukemia is shown. Data are representative of at least 2 independent experiments; n=3 replicates per cell line per experiment. Data are represented as mean+/−SD. nsP>0.05; *P<0.05; **P<0.01; ***P<0.001.

FIGS. 7A-7D demonstrate the chemical underpinnings and therapeutic implications of Aldh3a2 depletion. FIGS. 7A-7B show levels of endogenous C16 and C18 alcohols (7A) as well as total alcohol levels (7B) in Aldh3a2-control and mutant leukemic cells. FIG. 7C shows growth kinetics of Aldh3a2-control and mutant N-GMPs treated with 4-HNE. FIG. 7D shows sublethally irradiated C57BL/6J mice were injected with Aldh3a2-control and mutant leukemia cells from primary leukemic mice. Forty eight hours after injection mice were treated with 3 doses of Poly(I):Poly(C) on alternate days and cytarabine and doxorubicin as described. Leukemia development was monitored. Kaplan-Meier survival curve of animals that developed leukemia is shown. Data are representative of at least 2 independent experiments; n=3 replicates per cell line per experiment (except for measurement of alcohols where 2 replicates were used). Data are represented as mean+/−SD. nsP>0.05; *P<0.05; **P<0.01; ***P<0.001.

FIG. 8 shows metabolism focused screen evaluated L-GMPs and N-GMPs filters and the algorithm applied to the shRNA screen.

FIGS. 9A-9D show models used to investigate Aldh3a2 depletion in vitro and in vivo FIG. 9A shows representative images of a methylcellulose assay showing colony number and morphology in Aldh3a2 depleted and control L-GMPs. FIG. 9B shows a schema for generating Aldh3a2^{fl/fl} mice. FIGS. 9C-9D show Q-PCR results showing expression of Aldh3a2 (FALDH) splice variants V and N in Aldh3a2-control and mutant leukemic cells, relative to Actin-B.

FIGS. 10A-10B show Aldh3a2 depletion favors normal hematopoiesis in a conditional mouse model. FIG. 10A shows relative peripheral blood (PB) reconstitution after transplantation, of recipient B6.SJL (CD45.1) mice transfused with whole BM cells from Aldh3a2-control or mutant mice (CD45.2⁺) competed with equal numbers of wild-type CD45.1 whole BM cells (n=10 and 9 recipients per group respectively). FIG. 10B shows contribution of Aldh3a2-control and mutant HSPCs to peripheral blood B cells (B220), myeloid cells (Macl, Grl) and T cells (CD3) 24 weeks after transplantation (n=10 and 9 recipients per group respectively).

FIGS. 11A-11D show Aldh3a2 depletion in human AML cell lines with shRNA. FIGS. 11A-11D show the effective knockdown of ALDH3A2 expression in MOLM-14 (11A), NOMO-1 (11B), HL-60 (11C), and NB4 cells (11D) with two independent shRNAs (ALDH3A2-sh-A and ALDH3A2-sh-B) compared to control shRNA. Data are representative of at least 2 independent experiments; n=3 replicates per cell line per experiment. Data are represented as mean+/−SD. ^{ns}P>0.05; *P<0.05; **P<0.01; ***P<0.001.

FIGS. 12A-12H provide the biochemistry of Aldh3a2 depletion. FIG. 12A shows the fatty acid alcohol cycle depicting the role of Aldh3a2 in converting alcohols and aldehydes to fatty acids. Depletion of the enzyme can lead to a depletion of fatty acids and accumulation of alcohols and aldehydes. FIG. 12B shows the ratio of oxidized to reduced glutathione in ALDH3A2-depleted THP-1 cells. FIG. 12C shows the ratio of NADPH/NADP⁺ as measured in ALDH3A2-depleted THP-1 cells. FIG. 12D shows growth kinetics of ALDH3A2-depleted THP-1 cells treated with N-Acetyl-L-Cysteine (NAC). FIG. 12E depicts a schema showing that in cells cultured in [1,2-¹³C2]glucose, the oxidative pentose phosphate pathway (PPP) generates singly ¹³C labeled (M1) intermediates and glycolysis generates doubly ¹³C labeled (M2) intermediates. FIG. 12F shows the lactate M1/M2 ratio in ALDH3A2-depleted THP-1 cells. FIG. 12G shows fatty acid methyl ester (FAME) analysis in L-GMPs and Aldh3a2 depleted (Aldh3a2-sh-1) L-GMPs. FIG. 12H shows the frequency of live Aldh3a2-control and mutant LSPCs three days after culture and treatment with EtOH and varying concentrations of oleic acid reveal maximum rescue of cells from death at 125 μM of oleic acid. Data are representative of at least 2 independent experiments; n=3 replicates per cell line per experiment. Data are represented as mean+/−SD. ^{ns}P>0.05; *P<0.05; **P<0.01; ***P<0.001.

FIGS. 13A-13G show the role of lipid peroxidation and Acs13 depletion in Aldh3a2 depleted leukemia phenotype. FIGS. 13A-13E show lipidomics results for Aldh3a2-control and mutant leukemia cells. Ratios of major species of lipids (mutant/control values) are shown. Linoleic acid (18:2) containing phosphatidylcholine (13A), phosphatidylethanolamine (13B), cardiolipins (13C) and phosphatidic acid (13D) species were reduced and lysophospholipids (13E) (lacking one fatty acid side chain after oxidation) mainly in the lysophosphatidic acid and lysophosphatidylcholine classes were increased. FIG. 13F illustrates Q-PCR results showing Acs13 expression in Aldh3a2- control and mutant leukemia cells shows no difference in the two groups. FIG. 13G shows sublethally irradiated C57BL/6J mice were injected with Aldh3a2-control and mutant leukemia cells, from primary leukemic mice, infected with lentivirus expressing Acs13 or scrambled sRNA. Forty eight hours after injection mice were injected with 3 doses of Poly(I):Poly(C) on alternate days and leukemia development was monitored. Kaplan-Meier survival curve of animals that developed leukemia is shown.

FIGS. 14A-14C show role of ferroptosis and apoptosis in Aldh3a2 depleted leukemia phenotype. FIGS. 14A-14C show the frequency of live Aldh3a2-control and mutant LSPCs over three days of culture and treatment with DMSO (control), ZVAD (14A), Ferrostatin (14B) and RSL3 (14C).

### DETAILED DESCRIPTION OF THE INVENTION

It has been surprisingly found that leukemic cells, but not their normal myeloid counterparts, depended on the aldehyde dehydrogenase 3a2 (Aldh3a2), a metabolic enzyme that oxidizes long-chain aliphatic aldehydes to prevent lipid, protein, DNA, and cellular oxidative damage. Aldh3a2 is distinctive in its substrates and its predicted structure from other ALDH family members. Aldehydes are by-products of increased oxidative phosphorylation and nucleotide synthesis in cancer and generated from lipid peroxides underlying the non-caspase dependent form of cell death, ferroptosis. Leukemic cell dependence on Aldh3a2 was seen across multiple mouse and human myeloid leukemias and reduction in Aldh3a2 expression improved survival in murine AML models. Aldh3a2 inhibition was synthetically lethal with glutathione peroxidase-4 (GPX4) inhibition, a known trigger of ferroptosis that by itself minimally affects AML cells. Thus, disclosed herein are therapeutic anti-cancer strategies for inhibiting Aldh3a2 to exploit the distinctive metabolic state of malignant cells.

Aldh3a2 (NCBI Gene ID 224; MIM: 609523) is an aldehyde dehydrogenase 3 family member. Aldehyde dehydrogenase isozymes are thought to play a major role in the detoxification of aldehydes generated by alcohol metabolism and lipid peroxidation. This gene product catalyzes the oxidation of long-chain aliphatic aldehydes to fatty acid.

### Methods of Inducing Cell Death of Cancer Cells

Some aspects of the disclosure are related to a method of inducing cell death of cancer cells in a population of cells comprising contacting the population of cells with an effective amount of a first agent inhibiting Aldh3a2 activity or expression and an effective amount of a second agent inducing ferroptosis. In some embodiments, the method also inhibits the growth of the cancer cells. In some embodiments, the method does not induce or does not substantially induce cell death of non-cancer cells. In some embodiments, the uncontacted cancer cells exhibit increased redox stress or excessive production of reactive oxygen species. In some embodiments, the cancer cells are leukemia cells. In some embodiments, the cancer cells are AML cells. In some embodiments, the cancer cells are human cancer cells. In some specific embodiments, the method is used to treat cancer (e.g., leukemia) in a patient by administration of the first and second agent to the patient. In some embodiments, both the first and second agents are small molecules. In some embodiments, the first agent is a short hairpin RNA (shRNA) inhibiting Aldh3a2 expression (e.g., Aldh3a2-sh-1 and/or Aldh3a2-sh-2) and the second agent is a GPX4 inhibitor selected from erastin, RSL3, ML162, and DPI10.

AML is a genetically heterogeneous disease of blood stem and myeloid progenitor cells, characterized by the accumulation of malignant blasts in the bone marrow that severely impairs normal blood formation. In spite of the heterogeneous nature of AML, the various subtypes seem to share some common pathways leading to leukemogenesis, and the hierarchical nature of the disease is generally well established (Lane, et al., Blood 1150-1157 (2009)). AML is one of the best characterized malignancies from a genetic viewpoint. Numerous genetic transformation events leading to leukemia have been characterized (Marcucci, et al., J. Clinical Oncology 29, 475-486 (2011); Pui, et al., J. Clinical Oncology 29, 551-65 (2011); and Burnett, et al., J. Clinical Oncology 29, 487-94 (2011)).

In some embodiments, the method disclosed herein is performed in vivo in a patient presenting with leukemia. In some embodiments, the leukemia is acute myeloid leukemia. As used herein, “acute myeloid leukemia” encompasses all forms of acute myeloid leukemia and related neoplasms according to the World Health Organization (WHO) classification of myeloid neoplasms and acute leukemia, including all of the following subgroups in their relapsed or refractory state: Acute myeloid leukemia with recurrent genetic abnormalities, such as AML with t(8;21)(q22;q22); RUNX1-RUNX1T1, AML with inv(16)(p13.1q22) or t(16;16)(p13.1;q22); CBFB-MYH11, AML with t(9;11)(p22;q23); MLLT3-MLL, AML with t(6;9)(p23;q34); DEK-NUP214, AML with inv(3)(q21 q26.2) or t(3;3)(q21;q26.2); RPN1-EVI1, AML (megakaryoblastic) with t(1;22)(p13;q13); RBM15-MKL1, AML with mutated NPM1, AML with mutated CEBPA; AML with myelodysplasia-related changes; therapy-related myeloid neoplasms; AML, not otherwise specified, such as AML with minimal differentiation, AML without maturation, AML with maturation, acute myelomonocytic leukemia, acute monoblastic/monocytic leukemia, acute erythroid leukemia (e.g., pure erythroid leukemia, erythroleukemia, erythroid/myeloid), acute megakaryoblastic leukemia, acute basophilic leukemia, acute panmyelosis with myelofibrosis; myeloid sarcoma; myeloid proliferations related to Down syndrome, such as transient abnormal myelopoiesis or myeloid leukemia associated with Down syndrome; and blastic plasmacytoid dendritic cell neoplasm. In some embodiments, the leukemia is MLL-AML. In some embodiments, the leukemia has an Flt3 mutation. In some embodiments, the leukemia has an Flt3 mutation and the ferroptosis inhibitor is Sorafenib.

While certain aspects of the present inventions concern the selective induction of cell death of leukemic cells and related methods of treating leukemia (e.g., acute myeloid leukemia), it should be understood that the efficacy of the compositions and methods disclosed herein are not limited to leukemia cells, but rather extend broadly to all cancer cell types (e.g., solid and non-solid tumors). For example, in certain embodiments, the inventions disclosed herein relate to methods of inducing cell death of cancer cells in a population of cells, the methods comprising contacting the population of cells with an effective amount of a first agent inhibiting Aldh3a2 activity or expression and an effective amount of a second agent inducing ferroptosis, thereby inducing cell death in the cancer cells in the cell population. Similarly, in certain embodiments, the inventions disclosed herein are directed to an anti-cancer composition, comprising administering to a subject an effective amount of a first agent inhibiting Aldh3a2 activity or expression and an effective amount of a second agent inducing ferroptosis, thereby treating cancer in the subject.

In any of the foregoing embodiments, cell death of the cancer cells is selectively induced (or the cancer is treated) by the compositions and methods disclosed herein without inducing or not substantially inducing cell death in non-cancer cells in the population of cells. For example, in certain embodiments, cell death is induced in at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or as much as 100% of the cancer cells in the population of cells.

For example, in certain embodiments, cell death is not induced in at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or as much as 100% of the non-cancer cells in the population of cells (e.g., non-leukemic blood cells).

For example, in certain embodiments, the expression of Aldh3a2 or the activity of a gene product of Aldh3a2 is inhibited by contacting the cell (e.g., cancer cell, leukemia cell) with a first agent and is decreased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or as much as 100%.

For example, in certain embodiments, ferroptosis is induced by contacting the cell (e.g., cancer cell, leukemia cell) with a second agent and is increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or as much as 100%.

In some embodiments, the cancer cells comprise leukemia cells. In certain embodiments, the cancer cells are from cancers selected from colon cancer, breast cancer, ovarian cancer, lung cancer, prostate cancer; cancers of the oral cavity and pharynx, esophagus, stomach, small intestine, large intestine, colon, rectum, liver and biliary passages; pancreas, bone, connective tissue, skin, cervix, uterus, corpus endometrium, testis, bladder, kidney and other urinary tissues; cancers of the eye, brain, spinal cord, and meninges, including glioblastoma; cancers of the thyroid and other endocrine glands; Hodgkin's disease, non-Hodgkin's lymphomas, multiple myeloma, leukemias and lymphomas; adrenocarcinoma, angiosarcoma, astrocytoma, acoustic neuroma, anaplastic astrocytoma, basal cell carcinoma, blastoglioma, chondrosarcoma, choriocarcinoma, chordoma, craniopharyngioma, cutaneous melanoma, cystadenocarcinoma, endotheliosarcoma, embryonal carcinoma, ependymoma, Ewing's tumor, epithelial carcinoma, fibrosarcoma, gastric cancer, genitourinary tract cancers, glioblastoma multiforme, head and neck cancer, hemangioblastoma, hepatocellular carcinoma, hepatoma, Kaposi's sarcoma, large cell carcinoma, leiomyosarcoma, leukemias, liposarcoma, lymphatic system cancer, lymphomas, lymphangiosarcoma, lymphangioendotheliosarcoma, medullary thyroid carcinoma, medulloblastoma, meningioma mesothelioma, myelomas, myxosarcoma neuroblastoma, neurofibrosarcoma, oligodendroglioma, osteogenic sarcoma, epithelial ovarian cancer, papillary carcinoma, papillary adenocarcinomas, paraganglioma, parathyroid tumours, pheochromocytoma, pinealoma, plasmacytomas, retinoblastoma, rhabdomyosarcoma, sebaceous gland carcinoma, seminoma, skin cancers, melanoma, small cell lung carcinoma, non-small cell lung carcinoma, squamous cell carcinoma, sweat gland carcinoma, synovioma, thyroid cancer, uveal melanoma, and Wilm's tumor.

Oxidative metabolism has been shown to be dysregulated in blood cancers, gastro-intestinal cancers and tumors harboring K-Ras, B-Raf mutations or Myc activation. Thus, in some embodiments, the cancer cells are from cancers harboring these cancer indications.

In some embodiments, the first agent is a polypeptide, nucleic acid, or small molecule. In some embodiments, the first agent is a short hairpin RNA (shRNA) inhibiting Aldh3a2 expression. In some embodiments, the second agent is a polypeptide, nucleic acid, or small molecule. In some embodiments, the second agent is Erastin, DPI2, BSO, SAS, lanperisone, SRS, RSL3, DPI7, DPI10, FIN56, sorafenib, or artemisinin. In some embodiments, the second agent is a Nrf2, LSH, TFR1, or xCT modulator. In some embodiments, the second agent is a glutathione peroxidase 4 (GPX4) inhibitor. In some embodiments, the GPX4 inhibitor is erastin, RSL3, ML162, or DPI10. In certain aspects, the contacting of the cancer cells occurs in vitro or ex vivo. In certain aspects, the contacting occurs in vivo (e.g., wherein the in vivo contact is in a subject, such as a human subject suffering from acute myeloid leukemia). In some embodiments, the first agent and second agent are contacted with the population of cells simultaneously or sequentially.

As used herein, a “GPX4 inhibitor” is an agent that enhances non-caspase dependent cell death. The agents that enhance non-caspase dependent cell death (e.g., GPX4 inhibitors) are not limited and may be any suitable agent. See, e.g., Hangauer et al. Drug-tolerant persister cancer cells are vulnerable to GPX4 inhibition. Nature 2017 Nov. 9; 551 (7679): 247-250.

In some embodiments, the first agent is a short hairpin RNA (shRNA) inhibiting Aldh3a2 expression. In some embodiments, the second agent is a polypeptide, nucleic acid, or small molecule. In some embodiments, the second agent is a glutathione peroxidase 4 (GPX4) inhibitor. In some embodiments, the GPX4 inhibitor is erastin, RSL3, ML162, or DPI10.

In some embodiments, the expression of Aldh3a2 or the activity of a gene product of Aldh3a2 is inhibited or decreased by contacting the cell with an agent and/or utilizing any known gene editing technique (e.g., CRISPR, TALEN, or ZFN). “Contacting”, “contacting a cell” and similar terms as used herein, refer to any means of introducing an agent (e.g., a nucleic acid, peptide, antibody, small molecule, etc.) into a target cell, including chemical and physical means, whether directly or indirectly or whether the agent physically contacts the cell directly or is introduced into an environment in which the cell is present. Contacting is intended to encompass methods of exposing a cell, delivering to a cell, or “loading” a cell with an agent by viral or non-viral vectors, wherein such agent is bioactive upon delivery or wherein such agent is processed intracellularly to an active form. The method of delivery will be chosen for the particular agent and use (e.g., disease being treated). Parameters that affect delivery, as is known in the medical art, can include, inter alia, the cell type affected, and cellular location. In some embodiments, contacting includes administering the agent to a subject.

“Ferroptosis” is used herein to refer to a type of programmed cell death dependent on iron and characterized by the accumulation of lipid peroxides, and is genetically and biochemically distinct from other forms of regulated cell death such as apoptosis.

“Agent” is used herein to broadly refer to any substance, compound (e.g., molecule), supramolecular complex, material, or combination or mixture thereof that is used as the first agent or second agent. In some aspects, an agent can be represented by a chemical formula, chemical structure, or sequence. Example of agents, include, e.g., small molecules, polypeptides, nucleic acids (e.g., RNAi agents, antisense oligonucleotide, aptamers), lipids, polysaccharides, peptide mimetics, analogs, etc. In general, agents may be obtained using any suitable method known in the art. The ordinary skilled artisan will select an appropriate method based, e.g., on the nature of the agent. An agent may be at least partly purified. In some embodiments an agent may be provided as part of a composition, which may contain, e.g., a counter-ion, aqueous or non-aqueous diluent or carrier, buffer, preservative, or other ingredient, in addition to the agent, in various embodiments. In some embodiments an agent may be provided as a salt, ester, hydrate, or solvate. In some embodiments an agent is cell-permeable, e.g., within the range of typical agents that are taken up by cells and acts intracellularly, e.g., within mammalian cells. Certain compounds may exist in particular geometric or stereoisomeric forms. Such compounds, including cis- and trans-isomers, E- and Z-isomers, R- and S-enantiomers, diastereomers, (D)-isomers, (L)-isomers, (−)- and (+)-isomers, racemic mixtures thereof, and other mixtures thereof are encompassed by this disclosure in various embodiments unless otherwise indicated. Certain compounds may exist in a variety or protonation states, may have a variety of configurations, may exist as solvates (e.g., with water (i.e. hydrates) or common solvents) and/or may have different crystalline forms (e.g., polymorphs) or different tautomeric forms. Embodiments exhibiting such alternative protonation states, configurations, solvates, and forms are encompassed by the present disclosure where applicable.

An “analog” of a first agent refers to another agent that is structurally and/or functionally similar to the first agent. A “structural analog” of a first agent is an analog that is structurally similar to the first agent. Unless otherwise specified, the term “analog” as used herein refers to a structural analog. A structural analog of an agent may have substantially similar physical, chemical, biological, and/or pharmacological propert(ies) as the agent or may differ in at least one physical, chemical, biological, or pharmacological property. In some embodiments at least one such property differs in a manner that renders the analog more suitable for a purpose of interest. In some embodiments a structural analog of an agent differs from the agent in that at least one atom, functional group, or substructure of the agent is replaced by a different atom, functional group, or substructure in the analog. In some embodiments, a structural analog of an agent differs from the agent in that at least one hydrogen or substituent present in the agent is replaced by a different moiety (e.g., a different substituent) in the analog.

In some embodiments, the agent is a nucleic acid. The term “nucleic acid” refers to polynucleotides such as deoxyribonucleic acid (DNA) and ribonucleic acid (RNA). The terms “nucleic acid” and “polynucleotide” are used interchangeably herein and should be understood to include double-stranded polynucleotides, single-stranded (such as sense or antisense) polynucleotides, and partially double-stranded polynucleotides. A nucleic acid often comprises standard nucleotides typically found in naturally occurring DNA or RNA (which can include modifications such as methylated nucleobases), joined by phosphodiester bonds. In some embodiments a nucleic acid may comprise one or more non-standard nucleotides, which may be naturally occurring or non-naturally occurring (i.e., artificial; not found in nature) in various embodiments and/or may contain a modified sugar or modified backbone linkage. Nucleic acid modifications (e.g., base, sugar, and/or backbone modifications), non-standard nucleotides or nucleosides, etc., such as those known in the art as being useful in the context of RNA interference (RNAi), aptamer, CRISPR technology, polypeptide production, reprogramming, or antisense-based molecules for research or therapeutic purposes may be incorporated in various embodiments. Such modifications may, for example, increase stability (e.g., by reducing sensitivity to cleavage by nucleases), decrease clearance in vivo, increase cell uptake, or confer other properties that improve the translation, potency, efficacy, specificity, or otherwise render the nucleic acid more suitable for an intended use. Various non-limiting examples of nucleic acid modifications are described in, e.g., Deleavey G F, et al., Chemical modification of siRNA. Curr. Protoc. Nucleic Acid Chem. 2009; 39:16.3.1-16.3.22; Crooke, S T (ed.) Antisense drug technology: principles, strategies, and applications, Boca Raton: CRC Press, 2008; Kurreck, J. (ed.) Therapeutic oligonucleotides, RSC biomolecular sciences. Cambridge: Royal Society of Chemistry, 2008; U.S. Pat. Nos. 4,469,863; 5,536,821; 5,541,306; 5,637,683; 5,637,684; 5,700,922; 5,717,083; 5,719,262; 5,739,308; 5,773,601; 5,886,165; 5,929,226; 5,977,296; 6,140,482; 6,455,308 and/or in PCT application publications WO 00/56746 and WO 01/14398. Different modifications may be used in the two strands of a double-stranded nucleic acid. A nucleic acid may be modified uniformly or on only a portion thereof and/or may contain multiple different modifications. Where the length of a nucleic acid or nucleic acid region is given in terms of a number of nucleotides (nt) it should be understood that the number refers to the number of nucleotides in a single-stranded nucleic acid or in each strand of a double-stranded nucleic acid unless otherwise indicated. An “oligonucleotide” is a relatively short nucleic acid, typically between about 5 and about 100 nt long. In some embodiments, the nucleic acid codes for Aldh3a2 or functional variants thereof.

The term “RNA interference” (RNAi) encompasses processes in which a molecular complex known as an RNA-induced silencing complex (RISC) reduces gene expression in a sequence-specific manner in, e.g., eukaryotic cells, e.g., vertebrate cells, or in an appropriate in vitro system. RISC may incorporate a short nucleic acid strand (e.g., about 16-about 30 nucleotides (nt) in length) that pairs with and directs or “guides” sequence-specific degradation or translational repression of RNA (e.g., mRNA) to which the strand has complementarity. The short nucleic acid strand may be referred to as a “guide strand” or “antisense strand”. An RNA strand to which the guide strand has complementarity may be referred to as a “target RNA”. A guide strand may initially become associated with RISC components (in a complex sometimes termed the RISC loading complex) as part of a short double-stranded RNA (dsRNA), e.g., a short interfering RNA (siRNA). The other strand of the short dsRNA may be referred to as a “passenger strand” or “sense strand”. The complementarity of the structure formed by hybridization of a target RNA and the guide strand may be such that the strand can (i) guide cleavage of the target RNA in the RNA-induced silencing complex (RISC) and/or (ii) cause translational repression of the target RNA. Reduction of expression due to RNAi may be essentially complete (e.g., the amount of a gene product is reduced to background levels) or may be less than complete in various embodiments. For example, mRNA and/or protein level may be reduced by 50%, 60%, 70%, 75%, 80%, 85%, 90%, 95%, or more, in various embodiments. As known in the art, the complementarity between the guide strand and a target RNA need not be perfect (100%) but need only be sufficient to result in inhibition of gene expression. For example, in some embodiments 1, 2, 3, 4, 5, or more nucleotides of a guide strand may not be matched to a target RNA. “Not matched” or “unmatched” refers to a nucleotide that is mismatched (not complementary to the nucleotide located opposite it in a duplex, i.e., wherein Watson-Crick base pairing does not take place) or forms at least part of a bulge. Examples of mismatches include, without limitation, an A opposite a G or A, a C opposite an A or C, a U opposite a C or U, a G opposite a G. A bulge refers to a sequence of one or more nucleotides in a strand within a generally duplex region that are not located opposite to nucleotide(s) in the other strand. “Partly complementary” refers to less than perfect complementarity. In some embodiments a guide strand has at least about 80%, 85%, or 90%, e.g., at least about 91%, 92%, 93%, 94%, 95%, 96%, 97%, 98%, 99%, or 100% sequence complementarity to a target RNA over a continuous stretch of at least about 15 nt, e.g., between 15 nt and 30 nt, between 17 nt and 29 nt, between 18 nt and 25 nt, between 19 nt and 23 nt, of the target RNA. In some embodiments at least the seed region of a guide strand (the nucleotides in positions 2-7 or 2-8 of the guide strand) is perfectly complementary to a target RNA. In some embodiments, a guide strand and a target RNA sequence may form a duplex that contains no more than 1, 2, 3, or 4 mismatched or bulging nucleotides over a continuous stretch of at least 10 nt, e.g., between 10-30 nt. In some embodiments a guide strand and a target RNA sequence may form a duplex that contains no more than 1, 2, 3, 4, 5, or 6 mismatched or bulging nucleotides over a continuous stretch of at least 12 nt, e.g., between 10-30 nt. In some embodiments, a guide strand and a target RNA sequence may form a duplex that contains no more than 1, 2, 3, 4, 5, 6, 7, or 8 mismatched or bulging nts over a continuous stretch of at least 15 nt, e.g., between 10-30 nt. In some embodiments, a guide strand and a target RNA sequence may form a duplex that contains no mismatched or bulging nucleotides over a continuous stretch of at least 10 nt, e.g., between 10-30 nt. In some embodiments, between 10-30 nt is 10, 11, 12, 13, 14, 15, 16, 17, 18, 19, 20, 21, 22, 23, 24, 25, 26, 27, 28, 29, or 30 nt.

As used herein, the term “RNAi agent” encompasses nucleic acids that can be used to achieve RNAi in eukaryotic cells. Short interfering RNA (siRNA), short hairpin RNA (shRNA), and microRNA (miRNA) are examples of RNAi agents. siRNAs typically comprise two separate nucleic acid strands that are hybridized to each other to form a structure that contains a double stranded (duplex) portion at least 15 nt in length, e.g., about 15-about 30 nt long, e.g., between 17-27 nt long, e.g., between 18-25 nt long, e.g., between 19-23 nt long, e.g., 15, 16, 17, 18, 19, 20, 21, 22, 23, 24, 25, 26, 27, 28, 29, or 30 nucleotides. In some embodiments the strands of an siRNA are perfectly complementary to each other within the duplex portion. In some embodiments the duplex portion may contain one or more unmatched nucleotides, e.g., one or more mismatched (non-complementary) nucleotide pairs or bulged nucleotides. In some embodiments either or both strands of an siRNA may contain up to about 1, 2, 3, or 4 unmatched nucleotides within the duplex portion. In some embodiments a strand may have a length of between 15-35 nt, e.g., between 17-29 nt, e.g., 19-25 nt, e.g., 21-23 nt. Strands may be equal in length or may have different lengths in various embodiments. In some embodiments, strands may differ by 1-10 nt in length. A strand may have a 5′ phosphate group and/or a 3′ hydroxyl (—OH) group. Either or both strands of an siRNA may comprise a 3′ overhang of, e.g., about 1-10 nt (e.g., 1-5 nt, e.g., 2 nt). Overhangs may be the same length or different in lengths in various embodiments. In some embodiments an overhang may comprise or consist of deoxyribonucleotides, ribonucleotides, or modified nucleotides or modified ribonucleotides such as 2′-O-methylated nucleotides, or 2′-O-methyl-uridine. An overhang may be perfectly complementary, partly complementary, or not complementary to a target RNA in a hybrid formed by the guide strand and the target RNA in various embodiments.

shRNAs are nucleic acid molecules that comprise a stem-loop structure and a length typically between about 40-150 nt, e.g., about 50-100 nt, e.g., about 60-80 nt. A “stem-loop structure” (also referred to as a “hairpin” structure) refers to a nucleic acid having a secondary structure that includes a region of nucleotides which are known or predicted to form a double strand (stem portion; duplex) that is linked on one side by a region of (usually) predominantly single-stranded nucleotides (loop portion). Such structures are well known in the art and the term is used consistently with its meaning in the art. A guide strand sequence may be positioned in either arm of the stem, i.e., 5′ with respect to the loop or 3′ with respect to the loop in various embodiments. As is known in the art, the stem structure does not require exact base-pairing (perfect complementarity). Thus, the stem may include one or more unmatched residues or the base-pairing may be exact, i.e., it may not include any mismatches or bulges. In some embodiments the stem is between 15-30 nt, e.g., between 17-29 nt, e.g., between 19-25 nt. In some embodiments the stem is between 15-19 nt. In some embodiments the stem is between 19-30 nt. The primary sequence and number of nucleotides within the loop may vary. Examples of loop sequences include, e.g., UGGU; ACUCGAGA; UUCAAGAGA. In some embodiments a loop sequence found in a naturally occurring miRNA precursor molecule (e.g., a pre-miRNA) may be used. In some embodiments a loop sequence may be absent (in which case the termini of the duplex portion may be directly linked). In some embodiments a loop sequence may be at least partly self-complementary. In some embodiments the loop is between 1 and 20 nt in length, e.g., 1-15 nt, e.g., 4-9 nt. The shRNA structure may comprise a 5′ or 3′ overhang. As known in the art, an shRNA may undergo intracellular processing, e.g., by the ribonuclease (RNase) III family enzyme known as Dicer, to remove the loop and generate an siRNA.

Mature endogenous miRNAs are short (typically 18-24 nt, e.g., about 22 nt), single-stranded RNAs that are generated by intracellular processing from larger, endogenously encoded precursor RNA molecules termed miRNA precursors (see, e.g., Bartel, D., Cell. 116(2):281-97 (2004); Bartel D P. Cell. 136(2):215-33 (2009); Winter, J., et al., Nature Cell Biology 11: 228-234 (2009). Artificial miRNA may be designed to take advantage of the endogenous RNAi pathway in order to silence a target RNA of interest. The sequence of such artificial miRNA may be selected so that one or more bulges is present when the artificial miRNA is hybridized to its target sequence, mimicking the structure of naturally occurring miRNA:mRNA hybrids. Those of ordinary skill in the art are aware of how to design artificial miRNA.

An RNAi agent that contains a strand sufficiently complementary to an RNA of interest so as to result in reduced expression of the RNA of interest (e.g., as a result of degradation or repression of translation of the RNA) in a cell or in an in vitro system capable of mediating RNAi and/or that comprises a sequence that is at least 80%, 90%, 95%, or more (e.g., 100%) complementary to a sequence comprising at least 10, 12, 15, 17, or 19 consecutive nucleotides of an RNA of interest may be referred to as being “targeted to” the RNA of interest. An RNAi agent targeted to an RNA transcript may also be considered to be targeted to a gene from which the transcript is transcribed.

In some embodiments an RNAi agent is a vector (e.g., an expression vector) suitable for causing intracellular expression of one or more transcripts that give rise to a siRNA, shRNA, or miRNA in the cell. Such a vector may be referred to as an “RNAi vector”. An RNAi vector may comprise a template that, when transcribed, yields transcripts that may form a siRNA (e.g., as two separate strands that hybridize to each other), shRNA, or miRNA precursor (e.g., pri-miRNA or pre-mRNA).

An RNAi agent may be produced in any of a variety of ways in various embodiments. For example, nucleic acid strands may be chemically synthesized (e.g., using standard nucleic acid synthesis techniques) or may be produced in cells or using an in vitro transcription system. Strands may be allowed to hybridize (anneal) in an appropriate liquid composition (sometimes termed an “annealing buffer”). An RNAi vector may be produced using standard recombinant nucleic acid techniques.

In some embodiments, an agent inhibiting Aldh3a2 activity or expression enhances degradation of a nucleotide sequence (e.g., mRNA) coding for an Aldh3a2 gene product, or enhances degradation of an Aldh3a2 gene product. In some embodiments, degradation may be enhanced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or as much as 100%. In some embodiments, degradation may be enhanced by at least 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, 20-fold, 50-fold, 100-fold, up to and including, for example, the complete degradation of a nucleotide sequence (e.g., mRNA) coding for an Aldh3a2 gene product or Aldh3a2 gene product. In some embodiments, the agent enhances degradation of a nucleotide sequence (e.g., mRNA) coding for an Aldh3a2 gene product via RNA interference (RNAi). In some embodiments, the agent enhances degradation of an Aldh3a2 gene product by enhancing ubiquitination-proteasome degradation pathway (UPD pathway) dependent degradation. In some embodiments, UPD pathway degradation enhanced with an agent that specifically ubiquitinates an Aldh3a2 gene product.

Most protein degradation by the proteasome occurs via the ubiquitination-proteasome degradation pathway (UPD pathway), a multistep enzymatic cascade in eukaryotes in which ubiquitin is conjugated via a lysine residue to target proteins for destruction. Proteins tagged with lysine-linked chains of ubiquitin are marked for degradation in the proteasome. Proteasome-mediated protein degradation, e.g., via the UPD pathway, allows cells to eliminate excess and misfolded proteins and regulates various biological processes, such as cell proliferation.

In some embodiments, the agent is a small molecule. The term “small molecule” refers to an organic molecule that is less than about 2 kilodaltons (kDa) in mass. In some embodiments, the small molecule is less than about 1.5 kDa, or less than about 1 kDa. In some embodiments, the small molecule is less than about 800 daltons (Da), 600 Da, 500 Da, 400 Da, 300 Da, 200 Da, or 100 Da. Often, a small molecule has a mass of at least 50 Da. In some embodiments, a small molecule is non-polymeric. In some embodiments, a small molecule is not an amino acid. In some embodiments, a small molecule is not a nucleotide. In some embodiments, a small molecule is not a saccharide. In some embodiments, a small molecule contains multiple carbon-carbon bonds and can comprise one or more heteroatoms and/or one or more functional groups important for structural interaction with proteins (e.g., hydrogen bonding), e.g., an amine, carbonyl, hydroxyl, or carboxyl group, and in some embodiments at least two functional groups. Small molecules often comprise one or more cyclic carbon or heterocyclic structures and/or aromatic or polyaromatic structures, optionally substituted with one or more of the above functional groups.

In some embodiments, the agent is a protein or polypeptide. The term “polypeptide” refers to a polymer of amino acids linked by peptide bonds. A protein is a molecule comprising one or more polypeptides. A peptide is a relatively short polypeptide, typically between about 2 and 100 amino acids (aa) in length, e.g., between 4 and 60 aa; between 8 and 40 aa; between 10 and 30 aa. The terms “protein”, “polypeptide”, and “peptide” may be used interchangeably. In general, a polypeptide may contain only standard amino acids or may comprise one or more non-standard amino acids (which may be naturally occurring or non-naturally occurring amino acids) and/or amino acid analogs in various embodiments. A “standard amino acid” is any of the 20 L-amino acids that are commonly utilized in the synthesis of proteins by mammals and are encoded by the genetic code. A “non-standard amino acid” is an amino acid that is not commonly utilized in the synthesis of proteins by mammals. Non-standard amino acids include naturally occurring amino acids (other than the 20 standard amino acids) and non-naturally occurring amino acids. An amino acid, e.g., one or more of the amino acids in a polypeptide, may be modified, for example, by addition, e.g., covalent linkage, of a moiety such as an alkyl group, an alkanoyl group, a carbohydrate group, a phosphate group, a lipid, a polysaccharide, a halogen, a linker for conjugation, a protecting group, a small molecule (such as a fluorophore), etc.

In some embodiments, the agent is a peptide mimetic. The terms “mimetic,” “peptide mimetic” and “peptidomimetic” are used interchangeably herein, and generally refer to a peptide, partial peptide or non-peptide molecule that mimics the tertiary binding structure or activity of a selected native peptide or protein functional domain (e.g., binding motif or active site). These peptide mimetics include recombinantly or chemically modified peptides, as well as non-peptide agents such as small molecule drug mimetics.

In some embodiments, the agent is encoded by a synthetic RNA (e.g., modified mRNAs). The synthetic RNA can encode any suitable agent described herein. Synthetic RNAs, including modified RNAs are taught in WO 2017075406, which is herein incorporated by reference. In some embodiments, the agent is a synthetic RNA.

In some embodiments, the expression of Aldh3a2 or the activity of a gene product of Aldh3a2 is modulated utilizing any known gene editing technique (e.g., CRISPR, TALEN, ZFN, etc.). In some embodiments, the expression of Aldh3a2 or the activity of a gene product of Aldh3a2 is modulated utilizing CRISPR.

In some embodiments, a catalytically inactive site specific nuclease and an effector domain capable of attaching a DNA, RNA, or protein to the nucleotide sequence is used as the agent. In some embodiments, the catalytically inactive site specific nuclease dCas (e.g., dCas9 or Cpf1) is used as the agent. The agent may reduce or increase expression of Aldh3a2 (e.g., via modulating methylation of genomic DNA involved in expression of Aldh3a2) or reduce or increase activity of Aldh3a2 (e.g., by modifying the coding sequence for Aldh3a2). In some embodiments, the agent is a dCas-transcription activator domain fusion protein that enhances transcription of Aldh3a2 in the presence of the appropriate guide sequence. A variety of CRISPR associated (Cas) genes or proteins which are known in the art can be modified to make a catalytically inactive site specific nuclease, the choice of Cas protein will depend upon the particular conditions of the method (e.g., ncbi.nlm.nih.gov/gene/?term=cas9). Specific examples of Cas proteins include Cas1, Cas2, Cas3, Cas4, Cas5, Cas6, Cas7, Cas8, Cas9 and Cas10. In a particular aspect, the Cas nucleic acid or protein used in the methods is Cas9. In some embodiments a Cas protein, e.g., a Cas9 protein, may be from any of a variety of prokaryotic species. In some embodiments a particular Cas protein, e.g., a particular Cas9 protein, may be selected to recognize a particular protospacer-adjacent motif (PAM) sequence. In certain embodiments a Cas protein, e.g., a Cas9 protein, may be obtained from a bacteria or archaea or synthesized using known methods. In certain embodiments, a Cas protein may be from a gram positive bacteria or a gram negative bacteria. In certain embodiments, a Cas protein may be from a Streptococcus, (e.g., a S. pyogenes, a S. thermophilus) a Cryptococcus, a Corynebacterium, a Haemophilus, a Eubacterium, a Pasteurella, a Prevotella, a VeiUonella, or a Marinobacter. In some embodiments nucleic acids encoding two or more different Cas proteins, or two or more Cas proteins, may be introduced into a cell, zygote, embryo, or animal, e.g., to allow for recognition and modification of sites comprising the same, similar or different PAM motifs.

In some embodiments, the Cas protein is Cpf1 protein or a functional portion thereof. In some embodiments, the Cas protein is Cpf1 from any bacterial species or functional portion thereof. In certain embodiments, a Cpf1 protein is a Francisella novicida U112 protein or a functional portion thereof, an Acidaminococcus sp. BV3L6 protein or a functional portion thereof, or a Lachnospiraceae bacterium ND2006 protein or a function portion thereof. Cpf1 protein is a member of the type V CRISPR systems. Cpf1 protein is a polypeptide comprising about 1300 amino acids. Cpf1 contains a RuvC-like endonuclease domain.

In some embodiments, a Cas9 nickase may be generated by inactivating one or more of the Cas9 nuclease domains. In some embodiments, an amino acid substitution at residue 10 in the RuvC I domain of Cas9 converts the nuclease into a DNA nickase. For example, the aspartate at amino acid residue 10 can be substituted for alanine (Cong et al, Science, 339:819-823). Other amino acids mutations that create a catalytically inactive Cas9 protein include mutating at residue 10 and/or residue 840. Mutations at both residue 10 and residue 840 can create a catalytically inactive Cas9 protein, sometimes referred to herein as dCas9. For example, a D10A and a H840A Cas9 mutant is catalytically inactive.

As used herein an “effector domain” is a molecule (e.g., protein) that modulates the expression and/or activation of a genomic sequence (e.g., gene). The effector domain may have methylation activity or demethylation activity (e.g., DNA methylation or DNA demethylation activity). In some aspects, the effector domain targets one or both alleles of a gene. The effector domain can be introduced as a nucleic acid sequence and/or as a protein. In some aspects, the effector domain can be a constitutive or an inducible effector domain. In some aspects, a Cas (e.g., dCas) nucleic acid sequence or variant thereof and an effector domain nucleic acid sequence are introduced into a cell. In some aspects, the effector domain is fused to a molecule that associates with (e.g., binds to) Cas protein (e.g., the effector molecule is fused to an antibody or antigen binding fragment thereof that binds to Cas protein). In some aspects, a Cas (e.g., dCas) protein or variant thereof and an effector domain are fused or tethered creating a chimeric protein and are introduced into the cell as the chimeric protein. In some aspects, the Cas (e.g., dCas) protein and effector domain bind as a protein-protein interaction. In some aspects, the Cas (e.g., dCas) protein and effector domain are covalently linked. In some aspects, the effector domain associates non-covalently with the Cas (e.g., dCas) protein. In some aspects, a Cas (e.g., dCas) nucleic acid sequence and an effector domain nucleic acid sequence are introduced as separate sequences and/or proteins. In some aspects, the Cas (e.g., dCas) protein and effector domain are not fused or tethered.

In some embodiments, the catalytically inactive site specific nuclease can be guided to specific DNA sites by one or more RNA sequences (sgRNA) to modulate activity and/or expression of one or more genomic sequences (e.g., exert certain effects on transcription or chromatin organization, or bring specific kind of molecules into specific DNA loci, or act as sensor of local histone or DNA state). In specific aspects, fusions of a dCas9 tethered with all or a portion of an effector domain create chimeric proteins that can be guided to specific DNA sites by one or more RNA sequences to modulate or modify methylation or demethylation of one or more genomic sequences. As used herein, a “biologically active portion of an effector domain” is a portion that maintains the function (e.g. completely, partially, minimally) of an effector domain (e.g., a “minimal” or “core” domain). The fusion of the Cas9 (e.g., dCas9) with all or a portion of one or more effector domains created a chimeric protein.

Examples of effector domains include a transcription activation domain (e.g, Gal4, Oaf1, Leu3, Rtg3, Pho4, Gln3, Gcn4, p53, NFAT, NF-κB, or VP16 transcription activation domain), chromatin organizer domain, a remodeler domain, a histone modifier domain, a DNA modification domain, a RNA binding domain, a protein interaction input devices domain (Grunberg and Serrano, Nucleic Acids Research, 3 '8 (8): ‘2663-267’5 (2010)), and a protein interaction output device domain (Grunberg and Serrano, Nucleic Acids Research, 3 '8 (8): ‘2663-267’5 (2010)). In some aspects, the effector domain is a DNA modifier. Specific examples of DNA modifiers include 5hmc conversion from 5 mC such as Tet1 (Tet1CD); DNA demethylation by Tet1, ACID A, MBD4, Apobec1, Apobec2, Apobec3, Tdg, Gadd45a, Gadd45b, ROS1; DNA methylation by Dnmt1, Dnmt3a, Dnmt3b, CpG Methyltransferase M.SssI, and/or M.EcoHK31I. In specific aspects, an effector domain is Tet1. In other specific aspects, as effector domain is Dmnt3a. In some embodiments, dCas9 is fused to Tet1. In other embodiments, dCas9 is fused to Dnmt3a. Other examples of effector domains are described in PCT Application No. PCT/US2014/034387 and U.S. application Ser. No. 14/785,031, which are incorporated herein by reference in their entirety. Methods of using catalytically inactive site specific nuclease, effector domains for modifying a nucleotide sequence (e.g., genomic sequence), and sgRNA are taught in PCT/US2017/065918 filed 12 Dec. 2017, which is incorporated herein by reference.

### Methods of Treatment

Disclosed herein are methods of treating leukemia in a subject. Some aspects of the disclosure are related to a method of inducing cell death of cancer cells in a population of cells in vivo comprising administering to a subject in need thereof (e.g., a subject with cancer, a subject with leukemia) an effective amount of a first agent inhibiting Aldh3a2 activity or expression and an effective amount of a second agent inducing ferroptosis. In some embodiments, the method of treatment further comprises administration of a chemotherapeutic regimen or agent.

The agents are not limited and may be any agent described herein. In some embodiments, the agents comprise a polypeptide, amino acid, oligonucleotide, lipid, carbohydrate, hybrid molecule, peptide, nucleic acid, or small molecule.

Some aspects of the disclosure are related to a method of treating cancer in a subject in need thereof, comprising administering to the subject an effective amount of a first agent inhibiting Aldh3a2 activity or expression and a chemotherapeutic regimen. In some embodiments, the chemotherapeutic regimen is an induction chemotherapy treatment regimen to the subject. In some embodiments, the induction chemotherapy comprises administering an antimetabolite agent and an anthracycline agent to the subject. The cancer is not limited and may be any cancer described herein. In some embodiments, the cancer is leukemia. The subject or patient is not limited and may be any patient described herein. In some embodiments, the subject is human. The agent can be administered to the subject before the chemotherapy treatment regimen is administered to the subject, at the same time the chemotherapy treatment regimen is administered to the subject, after the chemotherapy treatment regimen is administered to the subject, or any combination of the above.

The terms “chemotherapy” and “chemotherapy regimen” refers to the use of drugs to treat cancer. A “chemotherapeutic agent” is used to connote a compound or composition that is administered in the treatment of cancer. Chemotherapeutic agents disclosed herein include, but are not limited to, alkylating agents such as thiotepa and cyclophosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytosine arabinoside, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenishers such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); taxoids, e.g. paclitaxel and docetaxel; chlorambucil; gemcitabine; 6-thioguanine; mercaptopurine; platinum analogs such as cisplatin and carboplatin; vinblastine; platinum; etoposide; ifosfamide; mitomycin C; mitoxantrone; vincristine; vinorelbine; navelbine; novantrone; teniposide; daunomycin; aminopterin; xeloda; ibandronate; CPT11; topoisomerase inhibitor RFS 2000; difluoromethylornithine; retinoic acid; esperamicins; capecitabine; and pharmaceutically acceptable salts, acids or derivatives of any of the above. Chemotherapeutic agents also include anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

In some embodiments, the chemotherapeutic agent is a topoisomerase inhibitor. Topoisomerase inhibitors are chemotherapy agents that interfere with the action of a topoisomerase enzyme (e.g., topoisomerase I or II). Topoisomerase inhibitors include, but are not limited to, doxorubicin HCl, daunorubicin citrate, mitoxantrone HCl, actinomycin D, etoposide, topotecan HCl, teniposide, and irinotecan, as well as pharmaceutically acceptable salts, acids, or derivatives of any of these.

In some embodiments, the chemotherapeutic agent is an anti-metabolite. An anti-metabolite is a chemical with a structure that is similar to a metabolite required for normal biochemical reactions, yet different enough to interfere with one or more normal functions of cells, such as cell division. Anti-metabolites include, but are not limited to, gemcitabine, fluorouracil, capecitabine, methotrexate sodium, ralitrexed, pemetrexed, tegafur, cytosine arabinoside, thioguanine, 5-azacytidine, 6-mercaptopurine, azathioprine, 6-thioguanine, pentostatin, fludarabine phosphate, and cladribine, as well as pharmaceutically acceptable salts, acids, or derivatives of any of these.

In certain embodiments, the chemotherapeutic agent is an antimitotic agent, including, but not limited to, agents that bind tubulin. In some embodiments, the agent is a taxane. In certain embodiments, the agent is paclitaxel or docetaxel, or a pharmaceutically acceptable salt, acid, or derivative of paclitaxel or docetaxel. In certain alternative embodiments, the antimitotic agent comprises a vinca alkaloid, such as vincristine, binblastine, vinorelbine, or vindesine, or pharmaceutically acceptable salts, acids, or derivatives thereof.

The chemotherapy treatment regimen can be administered to the subject over a period of hours, days, or months. In some embodiments more than one chemotherapeutic agent is administered to the subject. The chemotherapeutic agents can be administered at the same time throughout the period, or administered at different intervals within the period.

In certain embodiments the chemotherapy treatment regimen is an acute cytoreductive chemotherapy treatment regimen.

In certain embodiments the chemotherapy treatment regimen is an induction chemotherapy treatment regimen. The induction chemotherapy treatment regimen may be any regimen that is useful for inducing complete remission of acute myeloid leukemia in a subject. In some embodiments, the induction chemotherapy comprises administering an antimetabolite agent (e.g., cytarabine) and an anthracycline agent (e.g., doxorubicin) to the subject. In some embodiments, the antimetabolite agent comprises cytarabine. In some embodiments, the anthracycline agent comprises doxorubicin. The induction chemotherapy treatment regimen can be administered to the subject over a period of hours, days, or months. The chemotherapeutic agents used in the induction chemotherapy treatment regimen can be administered at the same time throughout the period, or administered at different intervals within the period. In some embodiments, the induction chemotherapy comprises administering cytarabine and doxorubicin to the subject for a period of 5 to 7 days. In some embodiments, the induction chemotherapy comprises administering cytarabine and doxorubicin to the subject for a period of 3 to 5 days, followed by administering cytarabine alone to the subject for a period of 2 to 3 days.

An “effective amount” or “effective dose” of an agent (or composition containing such an agent) refers to the amount sufficient to achieve a desired biological and/or pharmacological effect, e.g., when delivered to a cell or organism according to a selected administration form, route, and/or schedule. As will be appreciated by those of ordinary skill in this art, the absolute amount of a particular agent or composition that is effective may vary depending on such factors as the desired biological or pharmacological endpoint, the agent to be delivered, the target tissue, etc. Those of ordinary skill in the art will further understand that an “effective amount” may be contacted with cells or administered in a single dose, or through use of multiple doses, in various embodiments. A biological effect may be, e.g., reducing expression or activity of one or more gene products, reducing activity of a metabolic pathway or reaction, or reducing cell proliferation or survival of cells.

A single additional agent or multiple additional agents or treatment modalities may be co-administered (at the same or differing time points and/or via the same or differing routes of administration and/or on the same or a differing dosing schedule).

As used herein, a “subject” is a mammal, including but not limited to a primate (e.g., a human), rodent (e.g., mouse or rat) dog, cat, horse, cow, pig, sheep, goat, or chicken. Preferred subjects are human subjects. The human subject may be a pediatric or adult subject. In some embodiments, the subject is elderly. In some embodiments, the subject is at least about 40 years old, at least about 45 years old, at least about 50 years old, at least about 55 years old, at least about 60 years old, at least about 65 years old, at least about 70 years old, at least about 75 years old, at least about 80 years old, at least about 85 years old, or at least about 90 years old. In some embodiments, the subject has been diagnosed with, is suspected of having, or is at risk of having a disease or disorder described herein.

As used herein, “treatment” or “treating”, in reference to a subject, includes amelioration, cure, and/or maintenance of a cure (i.e., the prevention or delay of relapse and/or reducing the likelihood of recurrence) of a disorder. Treatment after a disorder has started aims to reduce, ameliorate or altogether eliminate the disorder, and/or its associated symptoms, to prevent it from becoming worse, to slow the rate of progression, or to prevent the disorder from re-occurring once it has been initially eliminated (i.e., to prevent a relapse). Treating encompasses administration of an agent that may not have an effect on the disorder by itself but increases the efficacy of a second agent administered to the subject. A suitable dose and therapeutic regimen may vary depending upon the specific agents used, the mode of delivery of the compound, and whether it is used alone or in combination.

As used herein, a therapeutic that “inhibits” or “prevents” a disorder or condition is a compound that, in a statistical sample, reduces the occurrence of the disorder or condition in the treated sample relative to an untreated control sample, or delays the onset or reduces the severity of one or more symptoms of the disorder or condition relative to the untreated control sample.

The dosage, administration schedule and method of administering the agents are not limited. In certain embodiments a reduced dose may be used when two or more agents are administered in combination either concomitantly or sequentially. The absolute amount will depend upon a variety of factors including other treatment(s), the number of doses and the individual patient parameters including age, physical condition, size and weight. These are factors well known to those of ordinary skill in the art and can be addressed with no more than routine experimentation. In some embodiments, a maximum tolerated dose may be used, that is, the highest safe and tolerable dose according to sound medical judgment.

As used herein, pharmaceutical compositions comprise one or more agents or compositions that have therapeutic utility, and a pharmaceutically acceptable carrier, e.g., a carrier that facilitates delivery of agents or compositions. Agents and pharmaceutical compositions disclosed herein may be administered by any suitable means such as topically, orally, intranasally, subcutaneously, intramuscularly, intravenously, intra-arterially, parenterally, intraperitoneally, intrathecally, intratracheally, ocularly, sublingually, vaginally, rectally, dermally, or as an aerosol.

Also disclosed herein are pharmaceutical compositions which may be administered topically using one or more pharmaceutically acceptable carriers or vehicles. Preferably, the pharmaceutical compositions disclosed herein can be applied directly to the skin of a subject. Also disclosed herein are topically administered compositions such as creams, ointments, serums, oils, lotions, gels, suspensions and/or solutions or the like that maintain a desired stability (e.g., remain stable at room temperature for at least two years or following exposure to freeze/thaw cycling).

Depending upon the type of condition to be treated, compounds of the invention may, for example, be inhaled, ingested or administered by systemic routes. Thus, a variety of administration modes, or routes, are available. The particular mode selected will typically depend on factors such as the particular compound selected, the particular condition being treated and the dosage required for therapeutic efficacy. The methods described herein, generally speaking, may be practiced using any mode of administration that is medically acceptable, meaning any mode that produces acceptable levels of efficacy without causing clinically unacceptable adverse effects. Preferred modes of administration are parenteral and oral routes. The term “parenteral” includes subcutaneous, intravenous, intramuscular, intraperitoneal, and intrasternal injection, or infusion techniques. In some embodiments, inhaled medications are of particular use because of the direct delivery to the lung, for example in lung cancer patients. Several types of metered dose inhalers are regularly used for administration by inhalation. These types of devices include metered dose inhalers (MDI), breath-actuated MDI, dry powder inhaler (DPI), spacer/holding chambers in combination with MDI, and nebulizers. In some embodiments, agents are delivered by pulmonary aerosol. Other appropriate routes will be apparent to one of ordinary skill in the art.

### Compositions

Some aspects of the disclosure are related to an anti-cancer composition, comprising a first agent which inhibits Aldh3a2 activity or expression, and a second agent which induces ferroptosis. The agent may be any agent, or combination of agents, disclosed herein. In some embodiments, the composition is configured to be administered to a subject as a single dose comprising both agents. In some embodiments, the composition is configured to be administered to a subject as two doses, each comprising one of the agents.

In some embodiments, the first agent is a short hairpin RNA (shRNA) inhibiting Aldh3a2 expression. In some embodiments, the second agent is a polypeptide, nucleic acid, or small molecule. In some embodiments, the second agent is Erastin, DPI2, BSO, SAS, lanperisone, SRS, RSL3, DPI7, DPI10, FIN56, sorafenib, or artemisinin. In some embodiments, the second agent is a Nrf2, LSH, TFR1, or xCT modulator. In some embodiments, the second agent is a glutathione peroxidase 4 (GPX4) inhibitor. In some embodiments, the GPX4 inhibitor is erastin, RSL3, ML162, or DPI10. In certain aspects, the composition is formulated for administration by a mode selected from the group consisting of: topically, by injection, by intravenous injection, by inhalation, continuous release by depot or pump, and a combination thereof.

In addition to the active agent(s), the pharmaceutical compositions typically comprise a pharmaceutically-acceptable carrier. The term “pharmaceutically-acceptable carrier”, as used herein, means one or more compatible solid or liquid vehicles, fillers, diluents, or encapsulating substances which are suitable for administration to a human or non-human animal. In preferred embodiments, a pharmaceutically-acceptable carrier is a non-toxic material that does not interfere with the effectiveness of the biological activity of the active ingredients. The term “compatible”, as used herein, means that the components of the pharmaceutical compositions are capable of being comingled with an agent, and with each other, in a manner such that there is no interaction which would substantially reduce the pharmaceutical efficacy of the pharmaceutical composition under ordinary use situations. Pharmaceutically-acceptable carriers should be of sufficiently high purity and sufficiently low toxicity to render them suitable for administration to the human or non-human animal being treated.

Some examples of substances which can serve as pharmaceutically-acceptable carriers are pyrogen-free water; isotonic saline; phosphate buffer solutions; sugars such as lactose, glucose, and sucrose; starches such as corn starch and potato starch; cellulose and its derivatives, such as sodium carboxymethylcellulose, ethylcellulose, cellulose acetate; powdered tragacanth; malt; gelatin; talc; stearic acid; magnesium stearate; calcium sulfate; vegetable oils such as peanut oil, cottonseed oil, sesame oil, olive oil, corn oil and oil of theobrama; polyols such as propylene glycol, glycerin, sorbitol, mannitol, and polyethylene glycol; sugar; alginic acid; cocoa butter (suppository base); emulsifiers, such as the Tweens; as well as other non-toxic compatible substances used in pharmaceutical formulation. Wetting agents and lubricants such as sodium lauryl sulfate, as well as coloring agents, flavoring agents, excipients, tableting agents, stabilizers, antioxidants, and preservatives, can also be present. It will be appreciated that a pharmaceutical composition can contain multiple different pharmaceutically acceptable carriers.

A pharmaceutically-acceptable carrier employed in conjunction with the compounds described herein is used at a concentration or amount sufficient to provide a practical size to dosage relationship. The pharmaceutically-acceptable carriers, in total, may, for example, comprise from about 60% to about 99.99999% by weight of the pharmaceutical compositions, e.g., from about 80% to about 99.99%, e.g., from about 90% to about 99.95%, from about 95% to about 99.9%, or from about 98% to about 99%.

Pharmaceutically-acceptable carriers suitable for the preparation of unit dosage forms for oral administration and topical application are well-known in the art. Their selection will depend on secondary considerations like taste, cost, and/or shelf stability, which are not critical for the purposes of the subject invention, and can be made without difficulty by a person skilled in the art.

Pharmaceutically acceptable compositions (including cosmetic preparations) can include diluents, fillers, salts, buffers, stabilizers, solubilizers and other materials which are well-known in the art. The choice of pharmaceutically-acceptable carrier to be used in conjunction with the compounds of the present invention is basically determined by the way the compound is to be administered. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof in certain embodiments. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts. It will also be understood that a compound can be provided as a pharmaceutically acceptable pro-drug, or an active metabolite can be used. Furthermore, it will be appreciated that agents may be modified, e.g., with targeting moieties, moieties that increase their uptake, biological half-life (e.g., pegylation), etc.

The agents may be administered in pharmaceutically acceptable solutions, which may routinely contain pharmaceutically acceptable concentrations of salt, buffering agents, preservatives, compatible carriers, adjuvants, and optionally other therapeutic ingredients.

The agents may be formulated into preparations in solid, semi-solid, liquid or gaseous forms such as tablets, capsules, powders, granules, ointments, solutions, depositories, inhalants and injections, and usual ways for oral, parenteral or surgical administration. The invention also embraces pharmaceutical compositions which are formulated for local administration, such as by implants.

Compositions suitable for oral administration may be presented as discrete units, such as capsules, tablets, lozenges, each containing a predetermined amount of the active agent. Other compositions include suspensions in aqueous liquids or non-aqueous liquids such as a syrup, elixir or an emulsion.

For oral administration, compositions can be formulated readily by combining the active agent(s) with pharmaceutically acceptable carriers well known in the art. Such carriers enable the agents to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethylcellulose, and/or polyvinylpyrrolidone (PVP). If desired, disintegrating agents may be added, such as the cross linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Pharmaceutical preparations which can be used orally include push fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration. For buccal administration, the compositions may take the form of tablets or lozenges formulated in conventional manner.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Lower doses will result from other forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of compounds.

In certain embodiments, the vehicle is a biocompatible microparticle or implant that is suitable for implantation into the mammalian recipient. Exemplary bioerodible implants that are useful in accordance with this method are described in PCT International Application Publication No. WO 95/24929, entitled “Polymeric Gene Delivery System”, which reports on a biodegradable polymeric matrix for containing a biological macromolecule. The polymeric matrix may be used to achieve sustained release of the agent in a subject. In some embodiments, an agent described herein may be encapsulated or dispersed within a biocompatible, preferably biodegradable polymeric matrix. The polymeric matrix may be in the form of a microparticle such as a microsphere (wherein the agent is dispersed throughout a solid polymeric matrix) or a microcapsule (wherein the agent is stored in the core of a polymeric shell). Other forms of polymeric matrix for containing the agent include films, coatings, gels, implants, and stents. The size and composition of the polymeric matrix device is selected to result in favorable release kinetics in the tissue into which the matrix device is implanted. The size of the polymeric matrix device further is selected according to the method of delivery which is to be used, typically injection into a tissue or administration of a suspension by aerosol into the nasal and/or pulmonary areas. The polymeric matrix composition can be selected to have both favorable degradation rates and also to be formed of a material which is bioadhesive, to further increase the effectiveness of transfer when the device is administered to a vascular, pulmonary, or other surface. The matrix composition also can be selected not to degrade, but rather, to release by diffusion over an extended period of time.

Both non-biodegradable and biodegradable polymeric matrices can be used to deliver the agents of the invention to the subject. Biodegradable matrices are preferred. Such polymers may be natural or synthetic polymers. Synthetic polymers are preferred. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multivalent ions or other polymers.

In general, the agents may be delivered using the bioerodible implant by way of diffusion, or more preferably, by degradation of the polymeric matrix. Exemplary synthetic polymers which can be used to form the biodegradable delivery system include: polyamides, polycarbonates, polyalkylenes, polyalkylene glycols, polyalkylene oxides, polyalkylene terepthalates, polyvinyl alcohols, polyvinyl ethers, polyvinyl esters, poly-vinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene and polyvinylpyrrolidone.

Examples of non-biodegradable polymers include ethylene vinyl acetate, poly(meth)acrylic acid, polyamides, copolymers and mixtures thereof.

Examples of biodegradable polymers include synthetic polymers such as polymers of lactic acid and glycolic acid, polyanhydrides, poly(ortho)esters, polyurethanes, poly(butic acid), poly(valeric acid), and poly(lactide-cocaprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

Bioadhesive polymers of particular interest include bioerodible hydrogels described by H. S. Sawhney, C. P. Pathak and J. A. Hubell in Macromolecules, 1993, 26, 581-587, the teachings of which are incorporated herein, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

Other delivery systems can include time-release, delayed release or sustained release delivery systems. Such systems can avoid repeated administrations of the peptide, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono- di- and tri-glycerides; hydrogel release systems; silastic systems; peptide based systems; wax coatings; compressed tablets using conventional binders and excipients; partially fused implants; and the like. Specific examples include, but are not limited to: (a) erosional systems in which the platelet reducing agent is contained in a form within a matrix such as those described in U.S. Pat. Nos. 4,452,775, 4,675,189, and 5,736,152 and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation. Liposomes, for example, which may comprise phospholipids or other lipids, are nontoxic, physiologically acceptable carriers that may be used in some embodiments. Liposomes can be prepared according to methods known to those skilled in the art. In some embodiments, for example, liposomes may be prepared as described in U.S. Pat. No. 4,522,811. Liposomes, including targeted liposomes, pegylated liposomes, and polymerized liposomes, are known in the art (see, e.g., Hansen C B, et al., Biochim Biophys Acta. 1239(2):133-44, 1995; Torchilin V P, et al., Biochim Biophys Acta, 1511(2):397-411, 2001; Ishida T, et al., FEBS Lett. 460(1):129-33, 1999). In some embodiments, a lipid-containing particle may be prepared as described in any of the following PCT application publications, or references therein: WO/2011/127255; WO/2010/080724; WO/2010/021865; WO/2010/014895; WO2010147655.

Long-term release, as used herein, means that the implant is constructed and arranged to deliver therapeutic levels of the active agent for at least 30 days, and preferably 60 days. Long-term sustained release implants are well-known to those of ordinary skill in the art and include some of the release systems described above.

In some embodiments, it may be advantageous to formulate oral or parenteral compositions in dosage unit form for ease of administration and uniformity of dosage. Unit dosage form as used herein refers to physically discrete units suited as unitary dosages for the subject to be treated; each unit containing a predetermined quantity of active compound calculated to produce the desired therapeutic effect in association with the required pharmaceutical carrier.

Generally, treatment of a subject can include a single treatment or, in many cases, can include a series of treatments. A pharmaceutical composition can be administered at various intervals and over different periods of time as required, e.g., multiple times per day, daily, every other day, once or more a week for between about 1 to 10 weeks, between 2 to 8 weeks, between about 3 to 7 weeks, about 4, 5, or 6 weeks, etc. It will be appreciated that multiple cycles of administration may be performed. Numerous variations are possible. The skilled artisan will appreciate that certain factors can influence the dosage and timing required to effectively treat a subject, including but not limited to the severity of the disease or disorder, previous treatments, the general health and/or age of the subject, and other diseases present.

### Methods of Screening

In some embodiments, an inhibitor of Aldh3a2 expression (e.g., an expression product of the Aldh3a2 gene) is identified by contacting a cell with a test agent and measuring the level of Aldh3a2 mRNA or protein. The agent is identified as an inhibitor of Aldh3a2 expression if the agent reduces the level of Aldh3a2 mRNA or protein as compared to a reference level (e.g., a control cell).

In some embodiments, an inducer of ferroptosis is identified by contacting a cell with a test agent and measuring the level of ferroptosis. The agent is identified as an inducer of ferroptosis if the agent increases the level of ferroptosis as compared to a reference level (e.g., a control cell).

In some embodiments, the methods of screening test agents described herein further comprise contacting the identified Aldh3a2 inhibitor or ferroptosis inducer with a test cell and measuring proliferation and/or survival of the contacted test cell as compared to a control cell not contacted with the identified inhibitor.

In some embodiments a method of screening one or more test agents to identify an inhibitor of Aldh3a2 expression or activity or a ferroptosis inducer comprises a high-throughput transport assay (e.g., in vitro transport assay). In some aspects an artificial membrane (e.g., a liposome) may be utilized. In other aspects a bacterial system may be utilized (e.g., Gram-negative bacteria such as E. coli or Gram-positive bacteria such as B. subtilis or Lactococcus lactis). In some embodiments, Aldh3a2 may be reconstituted into a liposome and one or more test agents are applied to Aldh3a2. In some embodiments bacterial Lactococcus lactis cells are grown to express Aldh3a2 and the cells are contacted with one or more test agents.

In certain embodiments of any method described herein, the survival or proliferation of cells, e.g., test cells and/or control cells, is determined by an assay selected from: a cell counting assay, a replication labeling assay, a cell membrane integrity assay, a cellular ATP-based viability assay, a mitochondrial reductase activity assay, a caspase activity assay, an Annexin V staining assay, a DNA content assay, a DNA degradation assay, and a nuclear fragmentation assay. Exemplary assays include BrdU, EdU, or H3-Thymidine incorporation assays; DNA content assays using a nucleic acid dye, such as Hoechst Dye, DAPI, actinomycin D, 7-aminoactinomycin D or propidium iodide; cellular metabolism assays such as AlamarBlue, MTT, XTT, and CellTitre Glo; nuclear fragmentation assays; cytoplasmic histone associated DNA fragmentation assay; PARP cleavage assay; TUNEL staining; and Annexin staining. In some embodiments, gene expression analysis (e.g., microarray, cDNA array, quantitative RT-PCR, RNAse protection assay, RNA-Seq) may be used to measure the expression of genes whose products mediate or are correlated with cell cycle, cell survival (or cell death, e.g., apoptosis), and/or cell proliferation, as an indication of the effect of an agent on cell viability or proliferation. Alternately or additionally, expression of proteins encoded by such genes may be measured. In other embodiments, the activity of a gene, such as those disclosed herein, can be assayed in a compound screen. In some embodiments, cells are modified to comprise an expression vector that includes a regulatory region of a gene whose products mediate or are correlated with cell cycle, cell survival (or cell death), and/or cell proliferation operably linked to a sequence that encodes a reporter gene product (e.g., a luciferase enzyme), wherein expression of the reporter gene is correlated with transcriptional activity of the gene. In such embodiments, assessment of reporter gene expression (e.g., luciferase activity) provides an indirect method for assessing cell survival or proliferation. Those of ordinary skill in the art are aware of genes whose products mediate or are correlated with cell cycle, cell survival (or cell death), and/or cell proliferation.

In some embodiments the activity of an agent (e.g., a test agent) can be tested by contacting test cells and control cells that are in a co-culture. Co-cultures enable selective evaluation of the properties (e.g., survival or proliferation) of two or more populations of cells (e.g., test and control cells) in contact with an agent in a common growth chamber. Typically, each population of cells grown in co-culture will have an identifying characteristic that is detectable and distinct from an identifying characteristic of the other population(s) of cells in the co-culture. In some embodiments, the identifying characteristic comprises a level of expression of a fluorescent protein or other reporter protein or a protein expressed at the cell surface that could be detected using an antibody. Numerous fluorescent proteins are known in the art and may be used. Such proteins include, e.g., green, blue, yellow, red, orange, and cyan fluorescent proteins (FP). In some embodiments, test cells and control cells express different, distinguishable FPs, e.g., a red FP and a green FP, or other pairs of FPs that have different emission spectra. Other reporter proteins include, e.g., enzymes such as luciferase, beta-galactosidase, alkaline phosphatase, etc. However, other identifying characteristics known in the art may be suitable, provided that the identifying characteristic enables measurement (e.g., by FACS or other suitable assay method) of the level of survival or proliferation of each of the two or more populations of cells in the co-culture. A cell can be modified to have an identifying characteristic using methods known in the art, e.g., by introducing into the cell a nucleic acid construct encoding an FP (or other detectable protein) operably linked to a promoter. In some embodiments, a nucleic acid construct that encodes an RNAi agent that reduces expression of Aldh3a2 and a nucleic acid construct that encodes a FP or other detectable protein are incorporated into the same vector. In some embodiments, they may be in different vectors. In some embodiments, the construct(s) may be integrated into the genome of the cell.

Compositions, e.g., co-cultures, comprising at least some test cells (e.g., between 1% and 99% test cells) and at least some control cells (e.g., between 1% and 99% control cells), are disclosed herein. In some embodiments the percentage of test cells is between 10% and 90%. In other embodiments the percentage of test cells is between 20% and 80%. In some embodiments the percentage of test cells is between 30% and 70%. In some embodiments the percentage of test cells is between 40% and 60%, e.g., about 50%. In some embodiments the composition further comprises a test agent.

In some embodiments, test cells and control cells are maintained in separate vessels (e.g., separate wells of a microwell plate) under substantially identical conditions.

Assay systems comprising test cells, control cells, and one or more test compounds, e.g., 10, 100, 1000, 10,000, or more test agents, wherein the cells and test agents are arranged in one or more vessels in a manner suitable for assessing effect of the test compound(s) on the cells, are aspects of the invention. Typically, the vessels contain a suitable tissue culture medium, and the test compounds are present in the tissue culture medium. One of skill in the art can select a medium and culture environment appropriate for culturing a particular cell type.

In various embodiments the number of test agents is at least 10; 100; 1000; 10,000; 100,000; 250,000; 500,000 or more. In some embodiments test agents are tested in individual vessels, e.g., individual wells of a multiwell plate (sometimes referred to as microwell or microtiter plate or dish). In some embodiments a multiwell plate of use in performing an assay or culturing or testing cells or agents has 6, 12, 24, 96, 384, or 1536 wells. Cells (test cells and/or control cells) can be contacted with one or more test agents for varying periods of time and/or at different concentrations. In certain embodiments cells are contacted with test agent(s) for between 1 hour and 20 days, e.g., for between 12 and 48 hours, between 48 hours and 5 days, e.g., about 3 days, between 2 and 5 days, between 5 days and 10 days, between 10 days and 20 days, or any intervening range or particular value. Cells can be contacted with a test agent during all or part of a culture period. Cells can be contacted transiently or continuously. Test agents can be added to culture media at the time of replenishing the media and/or between media changes. If desired, test agent can be removed prior to assessing growth and/or survival. In some embodiments a test agent is tested at 1, 2, 3, 5, 8, 10 or more concentrations. Concentrations of test agent may range, for example, between about 1 nM and about 100 μM. For example, concentrations 1 nM, 10 nM, 50 nM, 100 nM, 500 nM, 1 μM, 5 μM, 10 μM, 50 μM, 100 μM (or any subset of the foregoing) may be used.

In some embodiments of any aspect or embodiment in the present disclosure relating to cells, a population of cells, cell sample, or similar terms, the number of cells is between 10 and 10¹³ cells. In some embodiments the number of cells may be at least about 10³, 10⁴, 10⁵, 10⁶, 10⁷, 10⁸, 10⁹, 10¹⁰, 10¹¹, 10¹² cells, or more. In some embodiments, the number of cells is between 10⁵ and 10¹² cells, e.g., at least 10⁶, 10⁷, 10⁸, 10⁹, 10¹⁰, 10¹¹, up to about 10¹² or about 10¹³. In some embodiments a screen is performed using multiple populations of cells and/or is repeated multiple times. In some embodiments, the number of cells is between 10⁵ and 10¹² cells, e.g., at least 10⁶, 10⁷, 10⁸, 10⁹, 10¹⁰, 10¹¹, up to about 10¹². In some embodiments smaller numbers of cells are of use, e.g., between 1-10⁴ cells. In some embodiments a population of cells is contained in an individual vessel, e.g., a culture vessel such as a culture plate, flask, or well. In some embodiments a population of cells is contained in multiple vessels. In some embodiments two or more cell populations are pooled to form a larger population.

In some embodiments, each of one or more test cells is contacted with a different concentration of, and/or for a different duration with, a test agent than at least one other test cell; and/or each of the one or more control cells is contacted with a different concentration of, and/or for a different duration with, the test agent than at least one other control cell.

In some embodiments, a high throughput screen (HTS) is performed. A high throughput screen can utilize cell-free or cell-based assays. High throughput screens often involve testing large numbers of compounds with high efficiency, e.g., in parallel. For example, tens or hundreds of thousands of compounds can be routinely screened in short periods of time, e.g., hours to days. Often such screening is performed in multiwell plates containing, at least 96 wells or other vessels in which multiple physically separated cavities or depressions are present in a substrate. High throughput screens often involve use of automation, e.g., for liquid handling, imaging, data acquisition and processing, etc. Certain general principles and techniques that may be applied in embodiments of a HTS of the present invention are described in Macarrón R & Hertzberg R P. Design and implementation of high-throughput screening assays. Methods Mol Biol., 565:1-32, 2009 and/or An W F & Tolliday N J., Introduction: cell-based assays for high-throughput screening. Methods Mol Biol. 486:1-12, 2009, and/or references in either of these. Useful methods are also disclosed in High Throughput Screening: Methods and Protocols (Methods in Molecular Biology) by William P. Janzen (2002) and High-Throughput Screening in Drug Discovery (Methods and Principles in Medicinal Chemistry) (2006) by Jorg Hüser.

The term “hit” generally refers to an agent that achieves an effect of interest in a screen or assay, e.g., an agent that has at least a predetermined level of modulating effect on cell survival, cell proliferation, gene expression, protein activity, or other parameter of interest being measured in the screen or assay. Test agents that are identified as hits in a screen may be selected for further testing, development, or modification. In some embodiments a test agent is retested using the same assay or different assays. Additional amounts of the test agent may be synthesized or otherwise obtained, if desired. Physical testing or computational approaches can be used to determine or predict one or more physicochemical, pharmacokinetic and/or pharmacodynamic properties of compounds identified in a screen. For example, solubility, absorption, distribution, metabolism, and excretion (ADME) parameters can be experimentally determined or predicted. Such information can be used, e.g., to select hits for further testing, development, or modification. For example, small molecules having characteristics typical of “drug-like” molecules can be selected and/or small molecules having one or more unfavorable characteristics can be avoided or modified to reduce or eliminate such unfavorable characteristic(s).

Additional compounds, e.g., analogs, that have a desired activity can be identified or designed based on compounds identified in a screen. In some embodiments structures of hit compounds are examined to identify a pharmacophore, which can be used to design additional compounds. An additional compound may, for example, have one or more altered, e.g., improved, physicochemical, pharmacokinetic (e.g., absorption, distribution, metabolism and/or excretion) and/or pharmacodynamic properties as compared with an initial hit or may have approximately the same properties but a different structure. For example, a compound may have higher affinity for the molecular target of interest, lower affinity for a non-target molecule, greater solubility (e.g., increased aqueous solubility), increased stability, increased bioavailability, oral bioavailability, and/or reduced side effect(s), modified onset of therapeutic action and/or duration of effect. An improved property is generally a property that renders a compound more readily usable or more useful for one or more intended uses. Improvement can be accomplished through empirical modification of the hit structure (e.g., synthesizing compounds with related structures and testing them in cell-free or cell-based assays or in non-human animals) and/or using computational approaches. Such modification can make use of established principles of medicinal chemistry to predictably alter one or more properties. An analog that has one or more improved properties may be identified and used in a composition or method described herein. In some embodiments a molecular target of a hit compound is identified or known. In some embodiments, additional compounds that act on the same molecular target may be identified empirically (e.g., through screening a compound library) or designed.

Data or results from testing an agent or performing a screen may be stored or electronically transmitted. Such information may be stored on a tangible medium, which may be a computer-readable medium, paper, etc. In some embodiments a method of identifying or testing an agent comprises storing and/or electronically transmitting information indicating that a test agent has one or more propert(ies) of interest or indicating that a test agent is a “hit” in a particular screen, or indicating the particular result achieved using a test agent. A list of hits from a screen may be generated and stored or transmitted. Hits may be ranked or divided into two or more groups based on activity, structural similarity, or other characteristics.

Once a candidate agent is identified, additional agents, e.g., analogs, may be generated based on it. An additional agent, may, for example, have increased cell uptake, increased potency, increased stability, greater solubility, or any improved property. In some embodiments a labeled form of the agent is generated. The labeled agent may be used, e.g., to directly measure binding of an agent to a molecular target in a cell. In some embodiments, a molecular target of an agent identified as described herein may be identified. An agent may be used as an affinity reagent to isolate a molecular target. An assay to identify the molecular target, e.g., using methods such as mass spectrometry, may be performed. Once a molecular target is identified, one or more additional screens maybe performed to identify agents that act specifically on that target.

Any of a wide variety of agents may be used as a test agent in various embodiments. For example, a test agent may be a small molecule, polypeptide, peptide, amino acid, nucleic acid, oligonucleotide, lipid, carbohydrate, or hybrid molecule. In some embodiments a nucleic acid used as a test agent comprises a siRNA, shRNA, antisense oligonucleotide, aptamer, or random oligonucleotide. In some embodiments a test agent is cell permeable or provided in a form or with an appropriate carrier or vector to allow it to enter cells.

Agents can be obtained from natural sources or produced synthetically. Agents may be at least partially pure or may be present in extracts or other types of mixtures. Extracts or fractions thereof can be produced from, e.g., plants, animals, microorganisms, marine organisms, fermentation broths (e.g., soil, bacterial or fungal fermentation broths), etc. In some embodiments, a compound collection (“library”) is tested. A compound library may comprise natural products and/or compounds generated using non-directed or directed synthetic organic chemistry. In some embodiments a library is a small molecule library, peptide library, peptoid library, cDNA library, oligonucleotide library, or display library (e.g., a phage display library). In some embodiments a library comprises agents of two or more of the foregoing types. In some embodiments oligonucleotides in an oligonucleotide library comprise siRNAs, shRNAs, antisense oligonucleotides, aptamers, or random oligonucleotides.

A library may comprise, e.g., between 100 and 500,000 compounds, or more. In some embodiments a library comprises at least 10,000, at least 50,000, at least 100,000, or at least 250,000 compounds. In some embodiments compounds of a compound library are arrayed in multiwell plates. They may be dissolved in a solvent (e.g., DMSO) or provided in dry form, e.g., as a powder or solid. Collections of synthetic, semi-synthetic, and/or naturally occurring compounds may be tested. Compound libraries can comprise structurally related, structurally diverse, or structurally unrelated compounds. Compounds may be artificial (having a structure invented by man and not found in nature) or naturally occurring. In some embodiments compounds that have been identified as “hits” or “leads” in a drug discovery program and/or analogs thereof. In some embodiments a library may be focused (e.g., composed primarily of compounds having the same core structure, derived from the same precursor, or having at least one biochemical activity in common). Compound libraries are available from a number of commercial vendors such as Tocris BioScience, Nanosyn, BioFocus, and from government entities such as the U.S. National Institutes of Health (NIH). In some embodiments a test agent is not an agent that is found in a cell culture medium known or used in the art, e.g., for culturing vertebrate, e.g., mammalian cells, e.g., an agent provided for purposes of culturing the cells. In some embodiments, if the agent is one that is found in a cell culture medium known or used in the art, the agent may be used at a different, e.g., higher, concentration when used as a test agent in a method or composition described herein.

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more nucleic acids, polypeptides, cells, species or types of organism, disorders, subjects, or combinations thereof, can be excluded.

Where the claims or description relate to a composition of matter, e.g., a nucleic acid, polypeptide, cell, or non-human transgenic animal, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”. “Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated”.

The terms “decrease,” “reduce,” “reduced,” “reduction,” “decrease,” and “inhibit” are all used herein generally to mean a decrease by a statistically significant amount relative to a reference. However, for avoidance of doubt, “reduce,” “reduction” or “decrease” or “inhibit” typically means a decrease by at least 10% as compared to a reference level and can include, for example, a decrease by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, up to and including, for example, the complete absence of the given entity or parameter as compared to the reference level, or any decrease between 10-99% as compared to the absence of a given treatment.

The terms “increased,” “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90%, or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or more as compared to a reference level.

As used herein the term “comprising” or “comprises” is used in reference to compositions, methods, and respective component(s) thereof, that are essential to the method or composition, yet open to the inclusion of unspecified elements, whether essential or not.

The term “consisting of” refers to compositions, methods, and respective components thereof as described herein, which are exclusive of any element not recited in that description of the embodiment.

### EXAMPLES

Metabolic alterations in cancer represent convergent effects of oncogenic mutations. It was hypothesized that a metabolism-restricted genetic screen, comparing normal primary mouse hematopoietic cells and their malignant counterparts in an ex vivo system mimicking the bone marrow microenvironment, would define distinctive vulnerabilities in AML.

Hematopoiesis is particularly well-suited for testing cell state-specific dependencies because of the reliable analytical tools available. Normal Granulocyte Macrophage Progenitors (N-GMPs) and their malignant counterpart Leukemia GMPs (L-GMPs) in the mixed lineage leukemia (MLL)-AF9 AML model provide such an opportunity.

A metabolism-restricted genetic screen was conducted comparing normal primary mouse hematopoietic cells and their malignant counterparts in the context of primary bone marrow stromal (BMS) co-cultures. MLL-AF9 mouse AML cells and their non-malignant counterpart (normal granulocyte-monocyte progenitors; N-GMPs) were transduced with shRNAs targeting 162 metabolic enzymes and co-cultured with bone marrow-derived stromal cells to approximate the niche influence on metabolism observed in vivo. Candidate metabolic vulnerabilities were selected based on the criteria that a minimum of 3 independent shRNA constructs targeting the same gene reduced cell number in AML cells but not GMPs. The top hit from this screen was the Aldh3a2. Proof-of-principle is provided that such a screen can identify unsuspected metabolic vulnerabilities in cancer stem/progenitor cells.

Aldh3a2 has a dependency of L-GMPs not seen in N-GMPs. It protects AML cells from oxidative cell death and Aldh3a2 inhibition improved leukemia outcomes in vivo without compromising normal hematopoiesis. It induces an iron-dependent form of cell death but is distinctive from peroxide cell killing (ferroptosis) induced by GPX4 inhibition. While GPX4 inhibition alone has only modest effects on AML, it is superadditive with Aldh3a2 inhibition in vitro and in vivo. Metabolically focused synthetic lethality is a potential treatment strategy.

In vivo metabolic dependencies of malignant (versus normal counterpart) cells can be defined by ex vivo screening. Aldh3a2 is synthetically lethal with GPX4 inhibition providing a combination therapy approach based solely on metabolic state and not specific oncogenic mutations.

### Example 1-Loss-of-Function Screen Reveals Aldh3a2 as a Metabolic Vulnerability in MLL-AF9 Driven AML

To assess metabolic dependencies in AML, the previously described MLL-AF9 retrovirally-induced AML model was used. This allows for isolation of leukemic granulocyte/monocyte progenitor cells (L-GMPs, also known as leukemic stem cells (LSCs)), which share the immunophenotype of normal GMPs (N-GMPs; Lin^{low}, Scal⁻, cKit⁺, CD34⁺, CD16/32⁺). The focus was on LSCs since they can survive induction chemotherapy and result in relapsed disease.

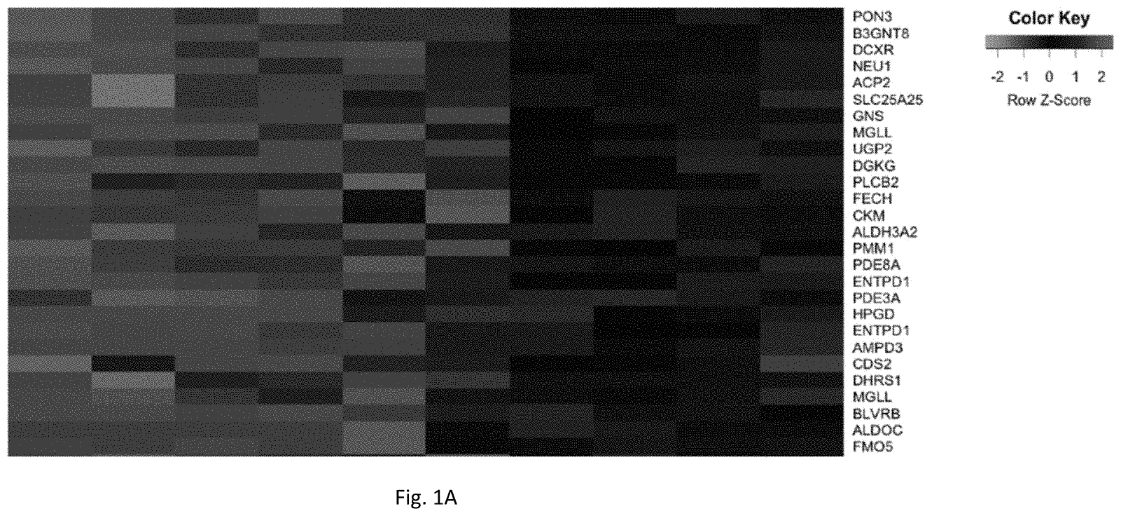

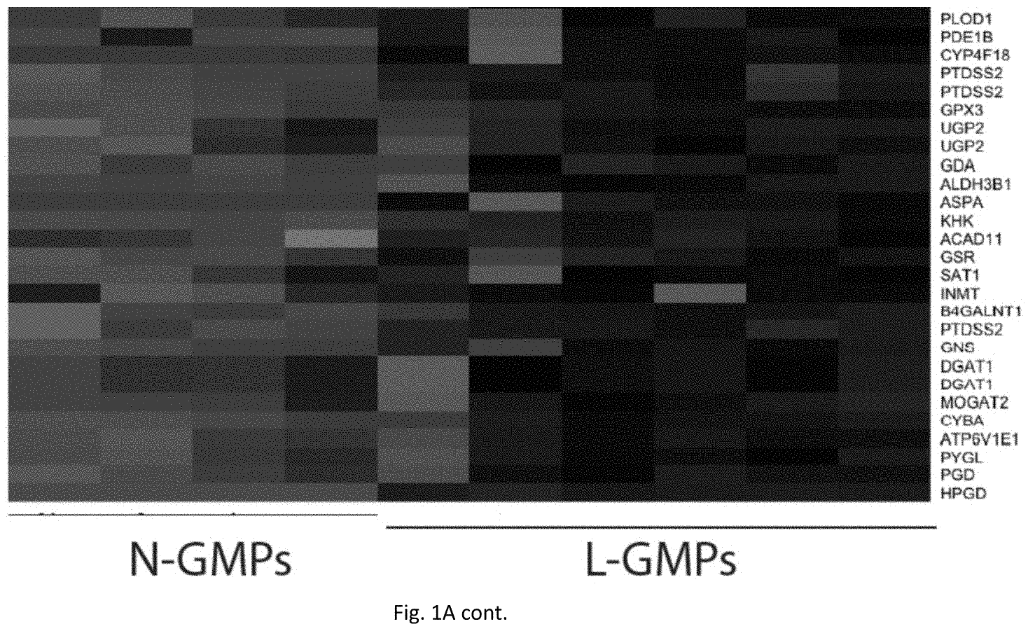

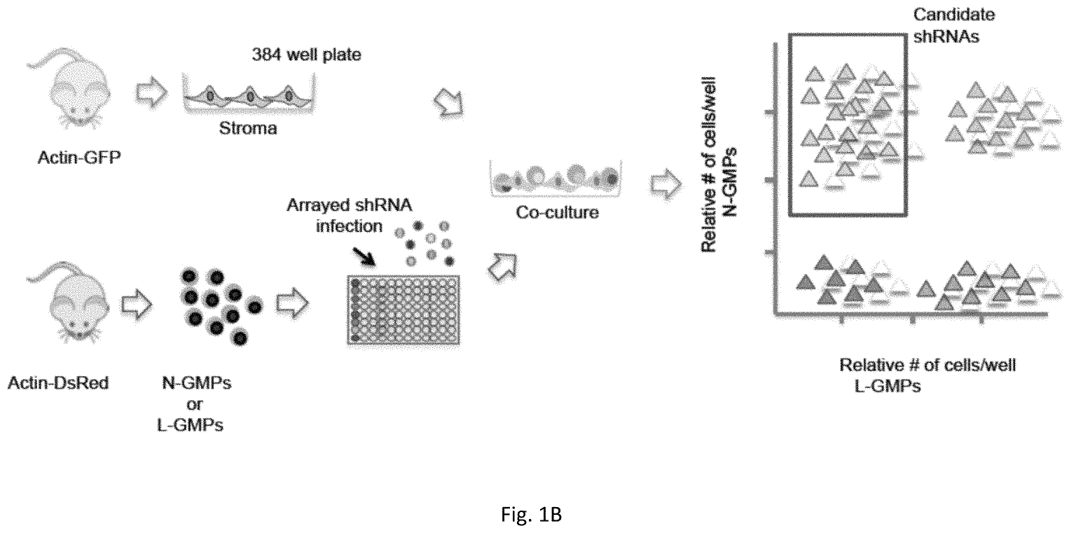

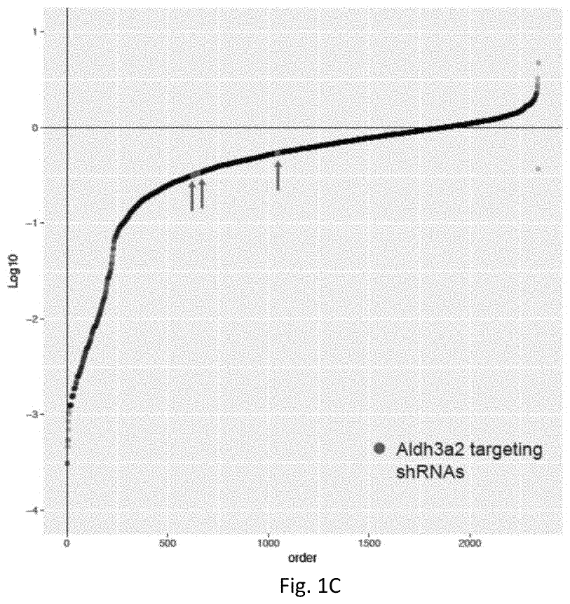

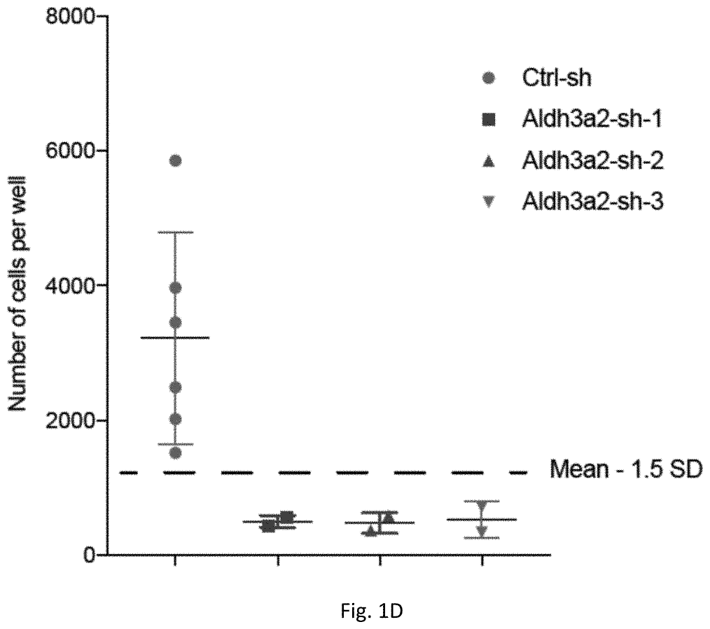

Bone marrow (BM) from actin-DsRed mice, treated with 5-flourouracil (5-FU) was isolated, retrovirally-transduced with MLL-AF9 (gift from Dr. Scott Armstrong) and transplanted into lethally-irradiated (9 Gy) C57BL/6J recipient mice (primary leukemia recipients). When these mice became moribund BM was isolated and injected into sublethally-irradiated (4.5 Gy) C57BL/6J mice (secondary leukemia recipients). When these mice became moribund L-GMPs were FACS sorted, plated in 384 well plates (800 cells/well) and transduced on an array format with shRNAs targeting 117 canonical rate limiting metabolic enzymes as selected from classic texts as well as 45 metabolic genes over-expressed in L-GMPs versus N-GMPs (FIG. 1A and Tables 1A and 1B below). N-GMPs from actin-DsRed mice were also transduced with the same shRNAs. An average of 5 shRNAs per gene obtained from the Broad Institute's RNAi Consortium were used. Puromycin selection (36 hours) was performed.

TABLE 1A
117 rate limiting enzymes curated from classic texts

| | Gene | Mouse |
|---|---|---|
| Gene name | Symbol | gene ID |
| 3-hydroxy-3-methylglutaryl-Coenzyme | Hmgcr | 15357 |
| A reductase | | |
| 6-phosphofructo-2-kinase/fructose-2,6- | Pfkfb1 | 18639 |
| biphosphatase 1 | | |
| 6-phosphofructo-2-kinase/fructose-2,6- | Pfkfb2 | 18640 |
| biphosphatase 2 | | |
| 6-phosphofructo-2-kinase/fructose-2,6- | Pfkfb3 | 170768 |
| biphosphatase 3 | | |
| 6-phosphofructo-2-kinase/fructose-2,6- | Pfkfb4 | 270198 |
| biphosphatase 4 | | |
| Acetyl-Coenzyme A acyltransferase 2 | Acaa2 | 52538 |
| (mitochondrial 3-oxoacyl-Coenzyme A thiolase) | | |
| Acetyl-Coenzyme A carboxylase alpha | Acaca | 107476 |
| Acetyl-Coenzyme A carboxylase beta | Acacb | 100705 |
| Adenylosuccinate synthetase like 1 | Adssl1 | 11565 |
| Adenylosuccinate synthetase, non muscle | Adss | 11566 |
| ADP-dependent glucokinase | Adpgk | 72141 |
| Arginase type II | Arg2 | 11847 |
| Arginase, liver | Arg1 | 11846 |
| Argininosuccinate synthetase 1 | Ass1 | 11898 |
| ATP citrate lyase | Acly | 104112 |
| ATPase inhibitory factor 1 | Atpif1 | 11983 |
| Brain glycogen phosphorylase | Pygp | 110078 |
| Carbamoyl-phosphate synthetase 1 | Cps1 | 227231 |
| Carbamoyl-phosphate synthetase 2, aspartate | Cad | 69719 |
| transcarbamylase, and dihydroorotase | | |
| Carnitine acetyl transferase | Crat | 12908 |
| Citrate lyase beta like | Clybl | 69634 |
| Citrate synthase | Cs | 12974 |
| Citrate synthase like | Csl | 71832 |
| Cytochrome c oxidase subunit IV isoform 1 | Cox4i1 | 12857 |
| Cytochrome c oxidase subunit IV isoform 2 | Cox4i2 | 84682 |
| Dihydrolipoamide S-acetyltransferase (E2 | Dlat | 235339 |
| component of pyruvate dehydrogenase complex) | | |
| Enoyl-CoenzymeA, hydratase/3-hydroxyacyl | Ehhadh | 74147 |
| coenzyme | | |
| Eph receptor B2 | Ephb2 | 13844 |
| Fructose bisphosphatase 1 | Fbp1 | 14121 |
| Fructose bisphosphatase 2 | Fbp2 | 14120 |
| fructose bisphosphatase 3 | Fbp3 | 14122 |
| Glucokinase | Gek | 103988 |
| Glucose 6 phosphatase, catalytic, 3 | G6pc3 | 68401 |
| Glucose 6-phosphatase, related sequence | G6pdrs | 107634 |
| glucose-6-phosphatase, catalytic | G6pc | 14377 |
| Glucose-6-phosphatase, catalytic, 2 | G6pc2 | 14378 |
| Glucose-6-phosphate dehydrogenase 2 | G6pd2 | 14380 |
| Glucose-6-phosphate dehydrogenase X-linked | G6pdx | 14381 |
| Glutamate-ammonia ligase (glutamine synthetase) | Glul | 14645 |
| Glutathione peroxidase 1 | Gpx1 | 14775 |
| Glutathione peroxidase 2 | Gpx2 | 14776 |
| Glutathione peroxidase 3 | Gpx3 | 14778 |
| Glutathione peroxidase 4 | Gpx4 | 625249 |
| Glutathione peroxidase 5 | Gpx5 | 14780 |
| Glutathione peroxidase 6 | Gpx6 | 75512 |
| Glutathione peroxidase 7 | Gpx7 | 67305 |
| Glutathione peroxidase 8 | Gpx8 | 69590 |
| Glycogen synthase1, muscle | Gys1 | 14936 |
| Glycogen synthase 2 | Gys2 | 232493 |
| Hadha hydroxyacyl-Coenzyme A dehydrogenase/ | Hadha | 97212 |
| 3-ketoacyl-Coenzyme A thiolase/enoyl-Coenzyme | | |
| A hydratase (trifunctional protein), alpha subunit | | |
| Heme oxygenase (decycling) 1 | Hmox1 | 15368 |
| Heme oxygenase (decycling) 2 | Hmox2 | 15369 |
| Hexokinase 1 | Hk1 | 15275 |
| Hexokinase 2 | Hk2 | 15277 |
| Hexokinase 3 | Hk3 | 212032 |
| Hexokinase-1 related sequence 1 | Hk1-rs1 | 110286 |
| Hydroxyacyl-Coenzyme A dehydrogenase/3- | Hadhb | 231086 |
| ketoacyl-Coenzyme A thiolase/enoyl-Coenzyme | | |
| A hydratase | | |
| (trifunctional protein), beta subunit | | |
| Inosine 5′-phosphate dehydrogenase 1 | Impdh1 | 23917 |
| Inosine 5′-phosphate dehydrogenase 2 | Impdh2 | 23918 |
| Isocitrate dehydrogenase 1 (NADP+), soluble | Idh1 | 15926 |
| Isocitrate dehydrogenase 2 (NADP+), | Idh2 | 269951 |
| mitochondrial | | |
| Isocitrate dehydrogenase 3 (NAD+) alpha | Idh3a | 67834 |
| Isocitrate dehydrogenase 3 (NAD+) beta | Idh3b | 170718 |
| Isocitrate dehydrogenase 3 (NAD+), gamma | Idh3g | 15929 |
| Ketohexokinase | Khk | 16548 |
| Lactate dehydrogenase A | Ldha | 16828 |
| Lactate dehydrogenase A-like 6B | Ldhal6b | 106557 |
| Lactate dehydrogenase B | Ldhb | 16832 |
| Lactate dehydrogenase C | Ldhc | 16833 |
| Lactate dehydrogenase D | Ldhd | 52815 |
| Lens protein with glutamine synthetase domain | Lgsn | 266744 |
| Liver glycogen phosphorylase | Pygl | 110095 |
| Malic enzyme 1, NADP(+)-dependent, cytosolic | Me1 | 17436 |
| Malic enzyme 2, NAD(+)-dependent, | Me2 | 107029 |
| mitochondrial | | |
| Malic enzyme 3, NADP(+)-dependent, | Me3 | 109264 |
| mitochondrial | | |
| Malic enzyme complex, mitochondrial | Mod2 | 110357 |
| Malic enzyme, related sequence | Mod-rs | 110087 |
| Mitogen-activated protein kinase 1 | Mapk1 | 26413 |
| Mitogen-activated protein 2 kinase 2 | Map2k2 | 26396 |
| Mitogen-activated protein 2 kinase 5 | Map2k5 | 23938 |
| mitogen-activated protein 2 kinase 2 | Map3k2 | 26405 |
| MLX interacting protein-like | Mlxipl | 58805 |
| Muscle glycogen phosphorylase | Pygm | 19309 |
| N-acetylglutamate synthase | Nags | 217214 |
| Ornithine transcarbamylase | Otc | 18416 |
| Oxoglutarate dehydrogenase (lipoamide) | Ogdh | 18293 |
| Phosphoenolpyruvate carboxykinase 1, cytosolic | Pck1 | 18534 |
| Phosphoenolpyruvate carboxykinase 2 | Pck2 | 74551 |
| Phosphofructokinase, liver, B-type | Pfkl | 18641 |
| Phosphofructokinase, muscle | Pfkm | 18642 |
| Phosphofructokinase, platelet | Pfkp | 56421 |
| Phosphofructokinase, polypeptide X | Pfkx | 18642 |
| Phosphogluconate dehydrogenase | Pgd | 110208 |
| Phosphoribosyl pyrophosphate amidotransferase | Ppat | 231327 |
| Phosphoribosyl pyrophosphate synthetase 1 | Prps1 | 19139 |
| Phosphoribosyl pyrophosphate synthetase 1-like 1 | Prps1l1 | 75456 |
| Phosphoribosyl pyrophosphate synthetase 2 | Prps2 | 110639 |
| Phosphoribosyl pyrophosphate synthetase- | Prpsap1 | 67763 |
| associated protein 1 | | |
| Protein kinase, cAMP dependent regulatory, type I | Prkar1b | 19085 |
| Protein kinase, cAMP dependent regulatory, | Prkar1a | 19084 |
| type I, alpha | | |
| Protein kinase, cAMP dependent regulatory, | Prkar2a | 19087 |
| type II alpha | | |
| Protein kinase, cAMP dependent regulatory, | Prkar2b | 19088 |
| type II beta | | |
| Protein kinase, cAMP dependent, catalytic, alpha | Prkaca | 18747 |
| Protein kinase, cAMP dependent, catalytic, beta | Prkacb | 18749 |
| Pyruvate carboxylase | Pcx | 18563 |
| Pyruvate dehydrogenase (lipoamide) beta | Pdhb | 68263 |
| Pyruvate dehydrogenase complex, component X | Pdhx | 27402 |
| Pyruvate dehydrogenase E1 alpha 1 | Pdha1 | 18597 |
| Pyruvate dehydrogenase E1 alpha 2 | Pdha2 | 18598 |
| Pyruvate kinase liver and red blood cell | Pklr | 18770 |
| Pyruvate kinase, muscle | Pkm2 | 18746 |
| Ribonucleotide reductase M1 | Rrm1 | 20133 |
| Ribonucleotide reductase M1, related sequence 13 | Rrm1- | 110820 |
| | rs13 | |
| Ribonucleotide reductase M2 | Rrm2 | 20135 |
| Ribonucleotide reductase M2 B | Rrm2b | 382985 |
| Solute carrier family 25 (mitochondrial carnitine/ | Slc25a20 | 57279 |
| acylcarnitine translocase), member 20 | | |
| UDP-glucose pyrophosphorylase 2 | Ugp2 | 216558 |

TABLE 1B
Genes over-expressed in L-GMPs versus N-GMPs (Krivstov et al)

| | | Mouse |
|---|---|---|
| | Gene | gene |
| Gene name | Symbol | ID |
| AcylcoA dehydrogenase 11 | Acad11 | 11370 |
| Lysosomal; acid phosphatase 2 | Acp2 | 11432 |
| Aldehyde dehydrogenase family 3, member A2 | Aldh3a2 | 11671 |
| Aldehyde dehydrogenase family 3, member B1 | Aldh3b1 | 67689 |
| aldolase C fructose bisphosphate | Aldoc | 11676 |
| adenosine monophosphate deaminase 3 | Ampd3 | 11717 |
| Aspartocyclase | Aspa | 11484 |
| ATPase, H+ transporting, lysosomal V1 subunit E1 | ATP6v1e1 | 11973 |
| UDP-GlcNAc:betaGal beta-1,3-N- | B3gnt8 | 232984 |
| acetylglucosaminyltransferase 8 | | |
| Beta-1,4-N-acetyl-galactosaminyl transferase 1 | B4galnt1 | 14421 |
| Biliverdin reductase B (flavin reductase (NADPH)) | Blvrb | 233016 |
| CDP-diacylglycerol synthase (phosphatidate | Cds2 | 110911 |
| cytidylyltransferase) 2 | | |
| Creatine kinase, muscle | Ckm | 12715 |
| Cytochrome b-245, alpha polypeptide | Cyba | 13057 |
| Cytochrome P450, family 4, subfamily f, | Cyp4f18 | 72054 |
| polypeptide 18 | | |
| Dicarbonyl L-xylulose reductase | Dcxr | 67880 |
| Diacylglycerol O-acyltransferase 1 | Dgat1 | 13350 |
| Diacylglycerol kinase, gamma | Dgkg | 110197 |
| Dehydrogenase/reductase (SDR family) member 1 | Dhrs1 | 52585 |
| Ectonucleoside triphosphate diphosphohydrolase 1 | Entpd1 | 12495 |
| Ferrochelatase | Fech | 14151 |
| Flavin containing monooxygenase 5 | Fmo5 | 14263 |
| Guanine deaminase | Gda | 14544 |
| Glucosamine (N-acetyl)-6-sulfatase | Gns | 75612 |
| Glutathione peroxidase 3 | Gpx3 | 14778 |
| Glutathione reductase | Gsr | 14782 |
| Hydroxyprostaglandin dehydrogenase 15 (NAD) | Hpgd | 15446 |
| Indolethylamine N-methyltransferase | Inmt | 21743 |
| Ketohexokinase | Khk | 16548 |
| Monoglyceride lipase | Mgll | 23945 |
| Monoacylglycerol O-acyltransferase 2 | Mogat2 | 233549 |
| Neuraminidase 1 | Neu1 | 18010 |
| Phosphodiesterase 1B, Ca2+-calmodulin dependent | Pde1b | 18574 |
| Phosphodiesterase 3A, cGMP inhibited | Pde3a | 54611 |
| Phosphodiesterase 8A | Pde8a | 18584 |
| Phosphogluconate dehydrogenase | Pgd | 110208 |
| Phospholipase C, beta 2 | Plcb2 | 18796 |
| Procollagen-lysine, 2-oxoglutarate 5-dioxygenase 1 | Plod1 | 18822 |
| Phosphomannomutase 2 | Pmm | 54128 |
| Paraoxonase 3 | Pon3 | 269823 |
| Phosphatidylserine synthase 2 | Ptdss2 | 27388 |
| Liver glycogen phosphorylase | Pygl | 110095 |
| Spermidine/spermine N1-acetyltransferase 1 | Sat1 | 20229 |
| Solute carrier family 25 (mitochondrial carrier, | Slc25a25 | 227731 |
| phosphate carrier), member 25 | | |
| UDP-glucose pyrophosphorylase 2 | Ugp2 | 216558 |

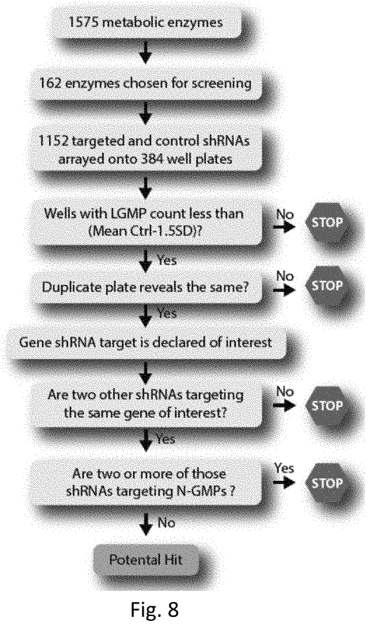

Stromal cells (CD105⁺, CD45⁻) from bone marrow (BM) of actin-GFP mice were plated at 2200 cells per well in 384 well plates. Transduced N-GMPs or L-GMPs were co-cultured with stroma to approximate the niche influence observed in vivo. Four days after stromal co-culture, cell content per well was evaluated by the high-content imaging system ImageXpress® Micro XLS and CellProfiler (FIG. 1B and FIG. 8). Changes in cell counts greater than 1.5 times the standard deviation above or below the mean number of cells infected with control virus were considered significant. Candidate metabolic vulnerabilities were selected based on the criteria that at least 3 independent shRNAs targeting the same gene reduced cell number in L-GMPs but not N-GMPs (FIGS. 1C-1D).

Six candidates were identified and Aldh3a2 was focused on. In recent years fatty acid metabolism has been investigated in AML and in normal hematopoiesis. However, little is known about fatty acid anabolism in LSCs; since Aldh3a2 is involved in long chain fatty acid metabolism this gene was chosen for further study.

### Example 2-Aldh3a2 is Essential for Leukemia Cells In Vitro and In Vivo

Aldehyde dehydrogenases comprise a family of enzymes that oxidize aldehydes to acids. Aldh3a2 is one member targeting longer chain aliphatic aldehydes.

Aldh3a2 is found in normal myeloid cells and in primary AML cells.

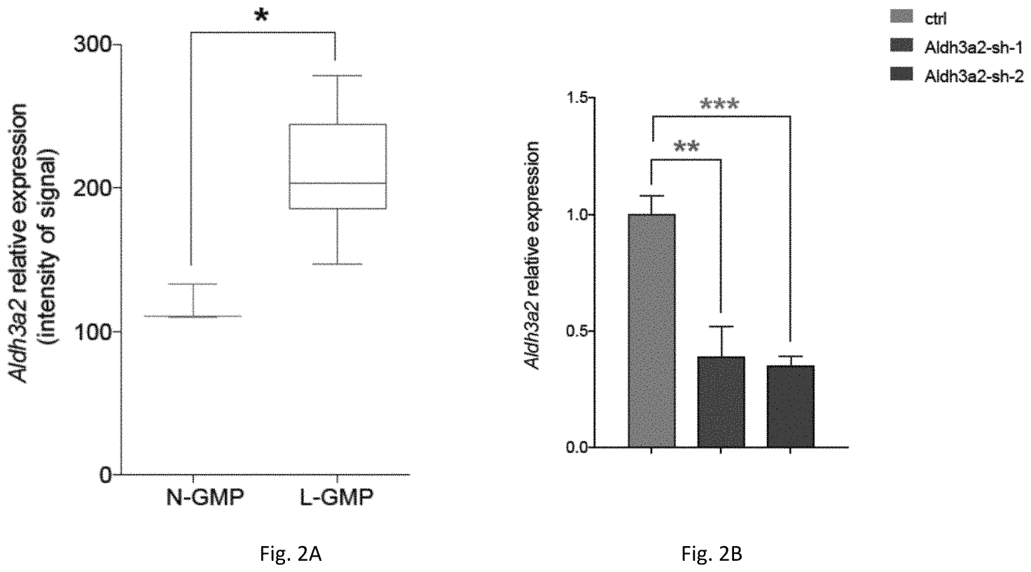

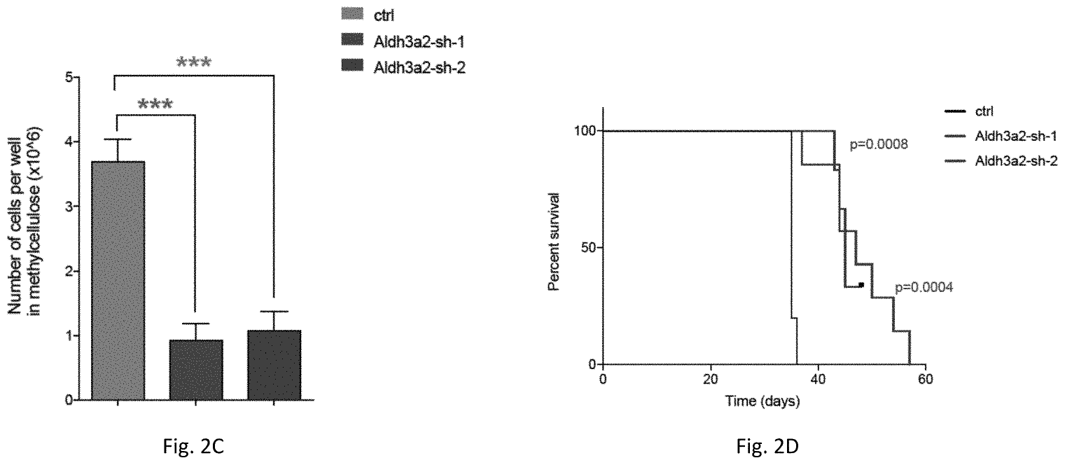

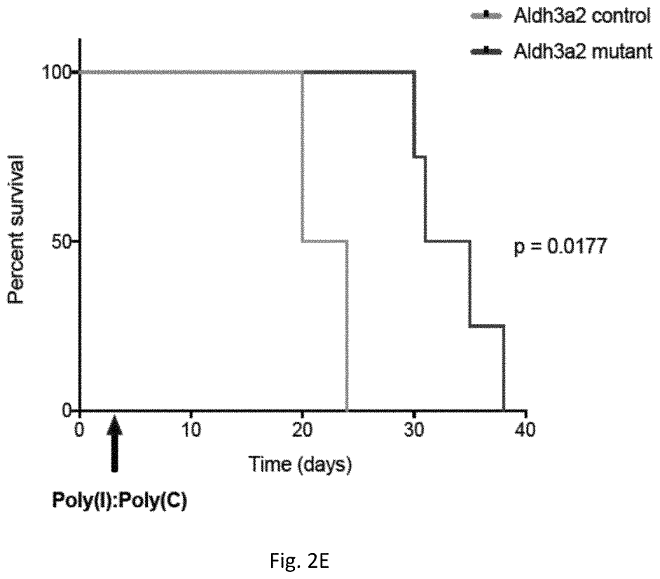

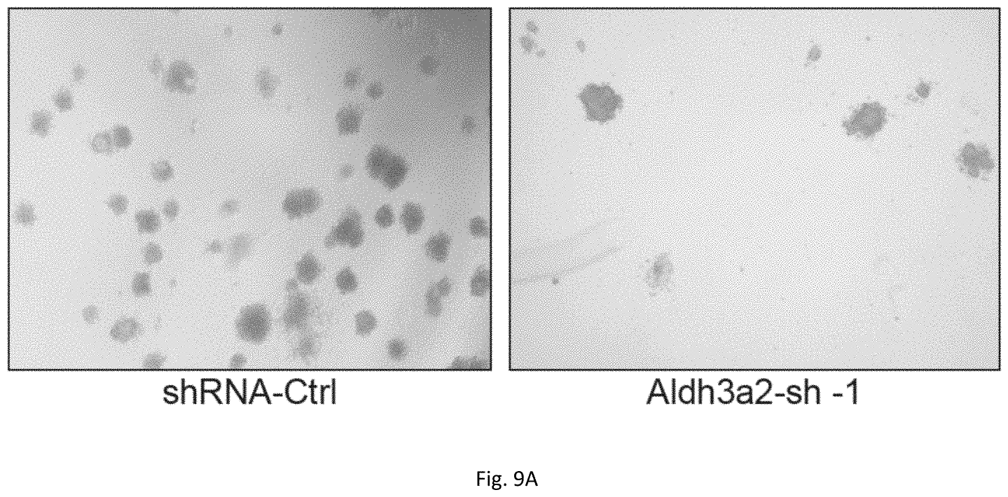

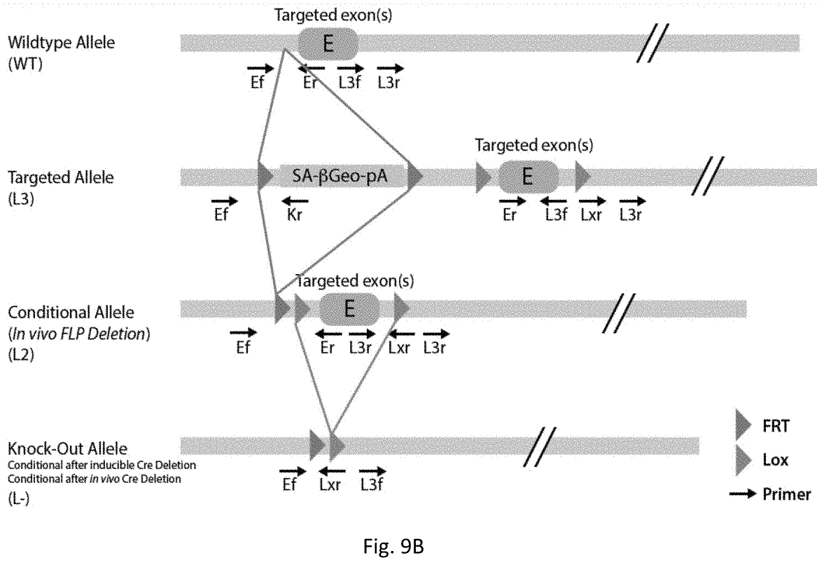

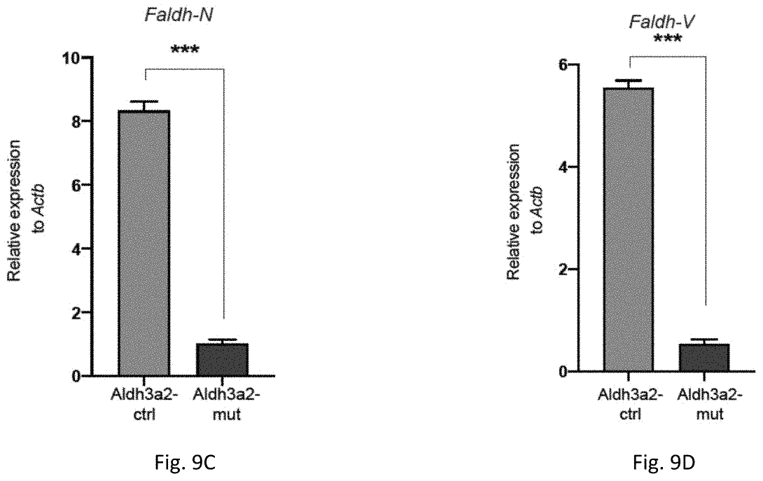

However, its functional role in normal and malignant hematopoiesis is unclear. To validate the screening results, Aldh3a2 expression was confirmed in primary L-GMPs (FIG. 2A) and evaluated two distinct shRNAs (Aldh3a2-sh-1, GCAGGAAATGCTGTGATTATA (SEQ ID NO: 1) and Aldh3a2-sh-2, CCTCTGGCTCTTTATGTATTT (SEQ ID NO: 2)) which decreased Aldh3a2 expression in L-GMPs (FIG. 2B). Aldh3a2 knockdown in L-GMPs decreased the number of cells in a methylcellulose assay (FIG. 2C and FIG. 9A) that quantifies growth on a per cell basis.

To evaluate Aldh3a2 knockdown in vivo, ten thousand L-GMPs expressing Aldh3a2-sh-1, Aldh3a2-sh-2 or control shRNA were transplanted into sublethally irradiated C57BL/6J recipient mice and followed over time. Animals receiving cells in which Aldh3a2 expression was reduced survive significantly longer than control animals (FIG. 2D).

To exclude off-target effects of shRNAs, a conditional mouse model was used in which exon 4 of the Aldh3a2 gene was flanked by loxP sites (FIG. 9B), and crossed these mice with mice expressing the Cre recombinase under control of the inducible Mx1 promoter (Mx1-cre) to allow deletion of Aldh3a2 in the hematopoietic system. Cells from FU treated Aldh3a2^{flx/flx}-Mx1-cre⁻ (Aldh3a2-control) and Aldh3a2^{flx/flx}-Mx1-cre⁺ (Aldh3a2-mutant) mice were infected with MLL-AF9 retrovirus expressing GFP downstream of an Internal Ribosomal Entry Site (IRES). Cells were transplanted into lethally irradiated C57BL/6J recipients. When mice became moribund, primary BM leukemic cells were isolated, expanded and transplanted into sublethally irradiated C57BL/6J mice. Two days after transplant cre-recombinase expression and Aldh3a2 deletion was induced by Polylnosinic-Polycytidylic acid (Poly(I)-Poly(C)). It was confirmed that this strategy deletes both splice variants of the Aldh3a2 gene by qPCR for Aldh3a2 (Faldh)-N and Aldh3a2 (Faldh)-V (FIGS. 9C-9D). Deletion of Aldh3a2 in the conditional mouse model led to a significant increased overall survival (FIG. 2E).

These data indicate that Aldh3a2 is important for L-GMPs and therefore constitutes a previously unrecognized vulnerability in MLL-AF9 driven leukemias.

### Example 3-Aldh3a2 is Dispensable for Normal Hematopoiesis

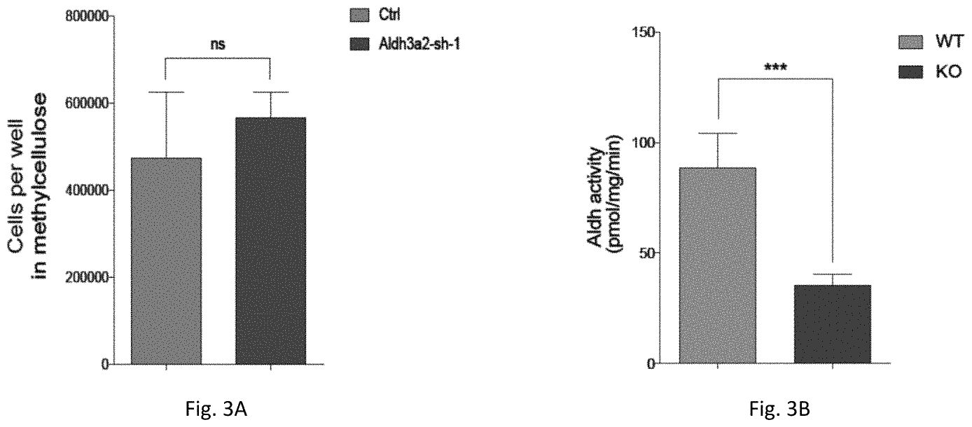

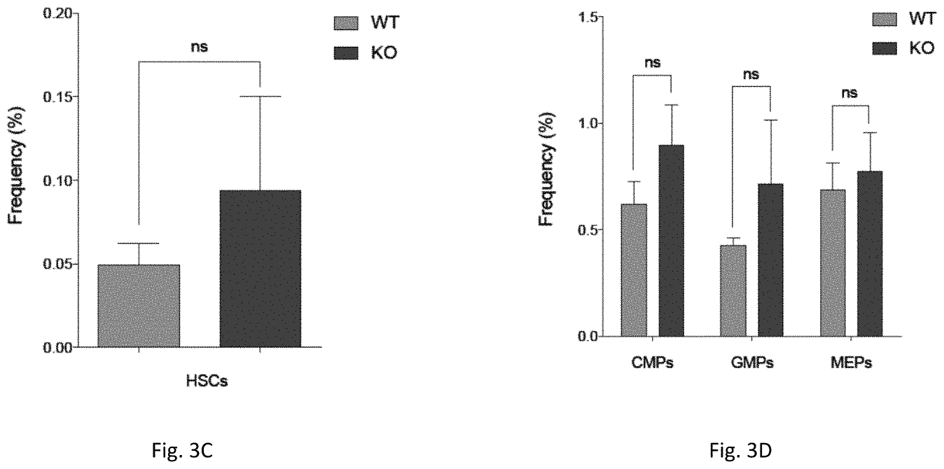

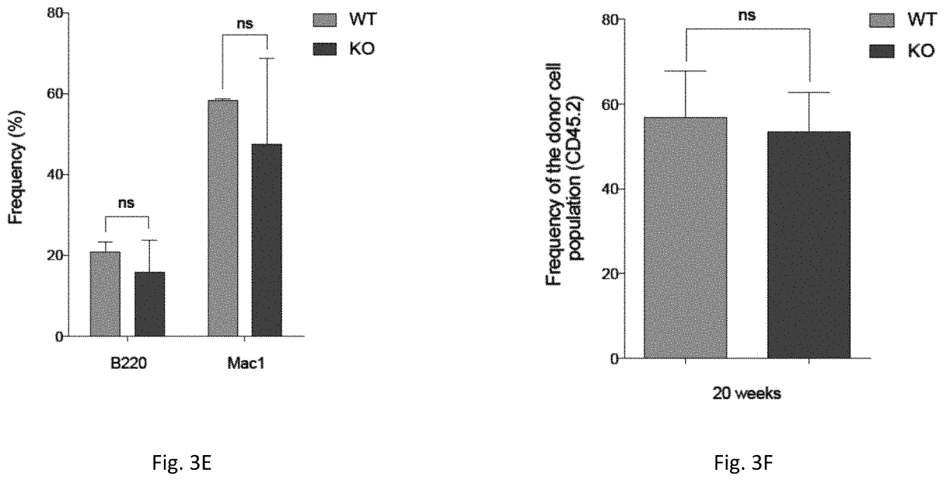

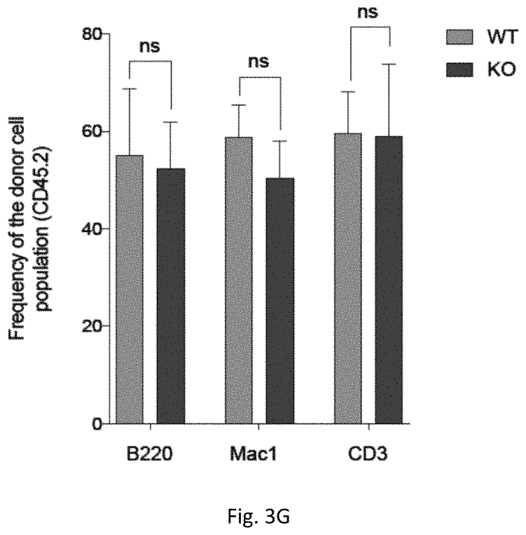

It was investigated whether Aldh3a2 is dispensable for normal hematopoiesis, as suggested from the screen. Aldh3a2 knockdown in N-GMPs did not decrease cell growth in a methylcellulose assay (FIG. 3A). Then a constitutive knockout mouse model (KO) was utilized where Aldh3a2 enzyme activity was decreased to less than 50 percent of wild-type (WT) control mice (FIG. 3B). (This model consists of a deletion of exon 4 of the Aldh3a2 gene which completely destroys Aldh3a2 enzyme activity but assays of cell homogenates measure total fatty aldehyde oxidizing activity including aldehyde dehydrogenases other than Aldh3a2). Immunophenotypic analysis in these mice revealed no significant differences in the percentage of hematopoietic stem cells or HSCs (Lin^{low}, cKit⁺, Scal⁺, CD48⁻, CD150⁺), progenitor cells (common myeloid progenitors: Lin^{low}, cKit⁺, Scal⁻, CD34⁺, CD16/32⁻; granulocyte monocyte precursor: Lin^{low}, cKit⁺, Scal⁻, CD34⁺, CD16/32⁺, and megakaryocyte erythroid precursors: Lin^{low}, cKit⁺, Scal⁻, CD34⁻, CD16/32⁻), as well as mature lymphoid (B220+ or CD3+) and myeloid cells (Mac-1+) in Aldh3a2 KO bone marrow (FIGS. 3C-3E). Furthermore, Aldh3a2 KO HSCs showed an equivalent capacity to reconstitute hematopoiesis and give rise to all blood cell lineages in a 1:1 competitive BM transplantation assay compared to Aldh3a2 WT cells (FIGS. 3F-3G).

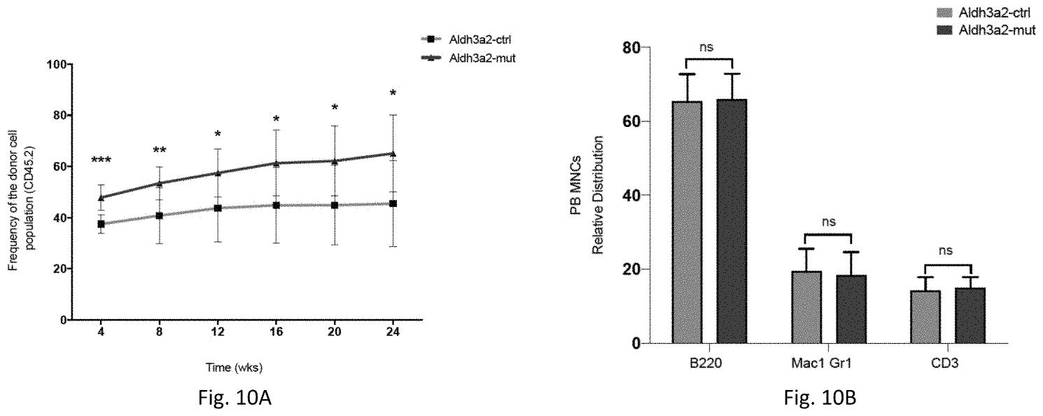

In transplantation assays using cells from the Aldh3a2 conditional mutant and control mice competing with equal numbers of congenic CD45.1 cells, a long term increase in the contribution to normal hematopoiesis from mutant cells was observed compared to control cells (FIG. 10A). Contribution of mutant and control cells to differentiated lineages was equivalent (FIG. 10B).

Taken together these results indicate that Aldh3a2 is dispensable for normal hematopoiesis.

### Example 4-Human Leukemia is Sensitive to ALDH3A2 Depletion

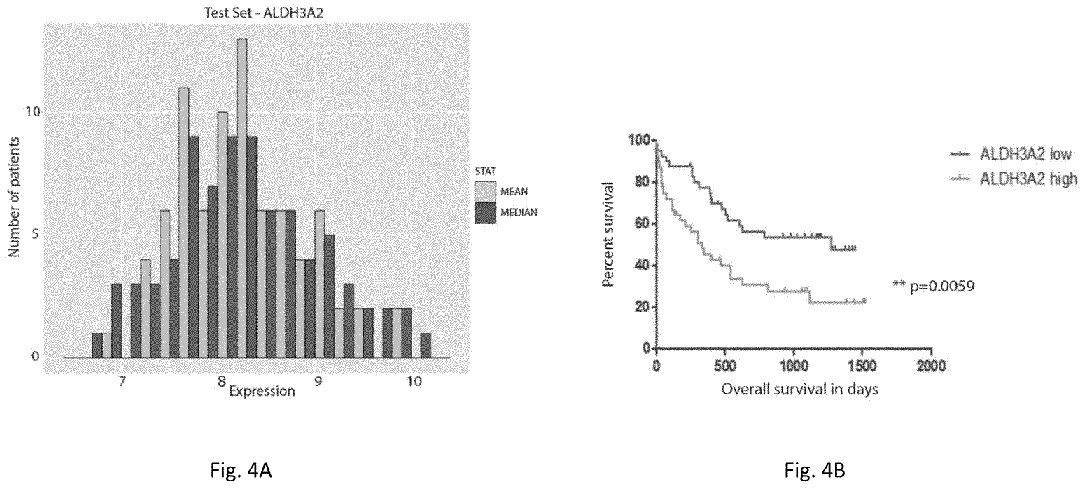

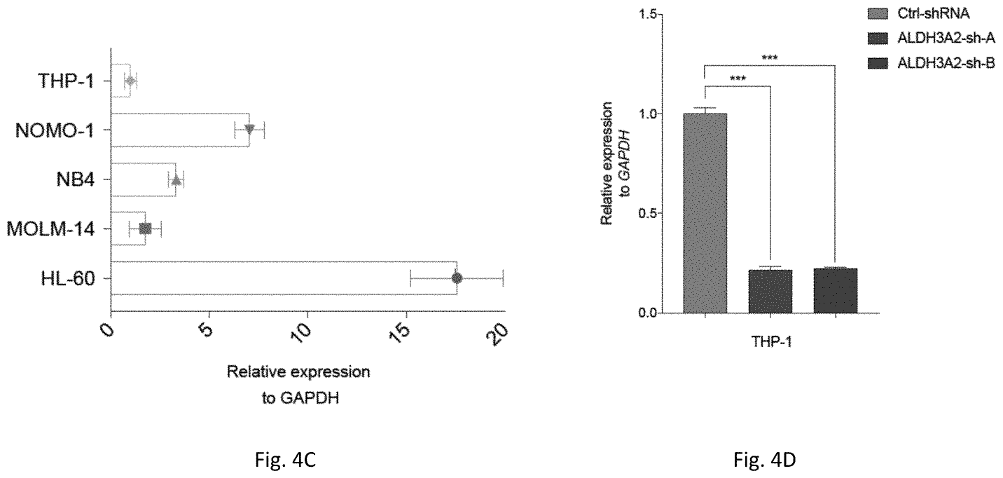

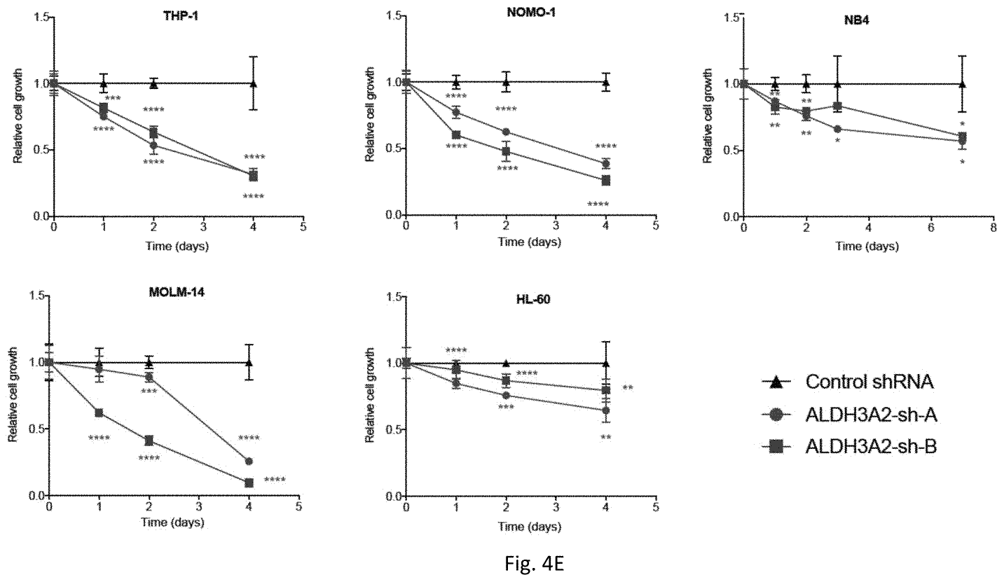

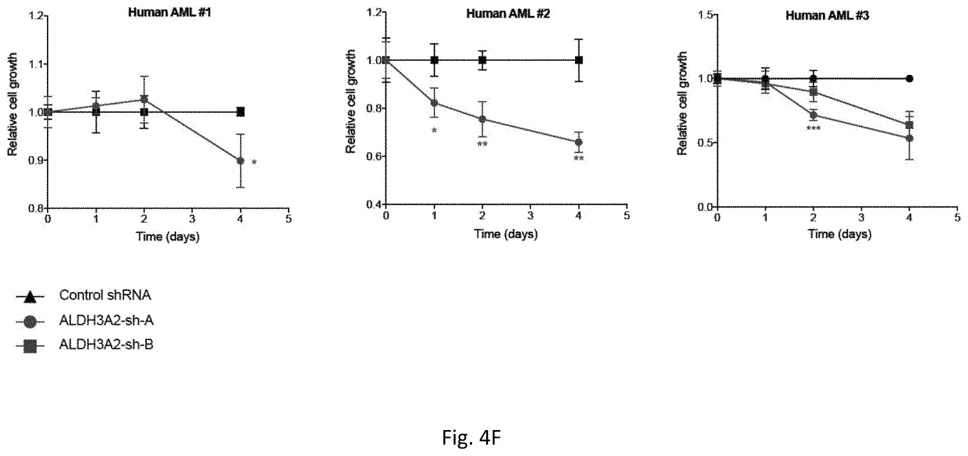

In order to determine whether ALDH3A2 is essential in human AML, the expression of ALDH3A2 in public gene expression databases was first investigated. Patients with karyotypically normal AML express a range of ALDH3A2 as measured by transcript levels in AML samples obtained at diagnosis (FIG. 4A). High levels of ALDH3A2 expression in this dataset was associated with a reduced overall survival compared to patients with lower ALDH3A2 expression (FIG. 4B).

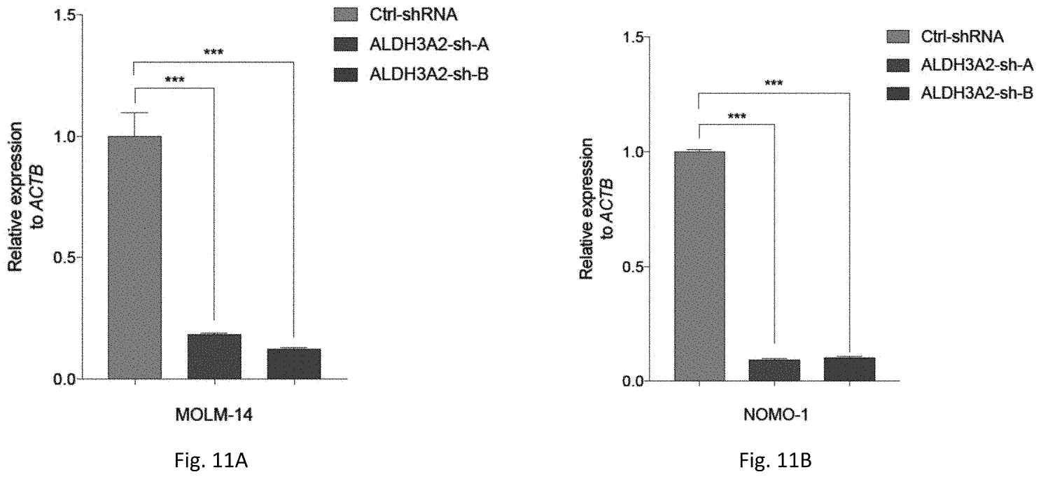

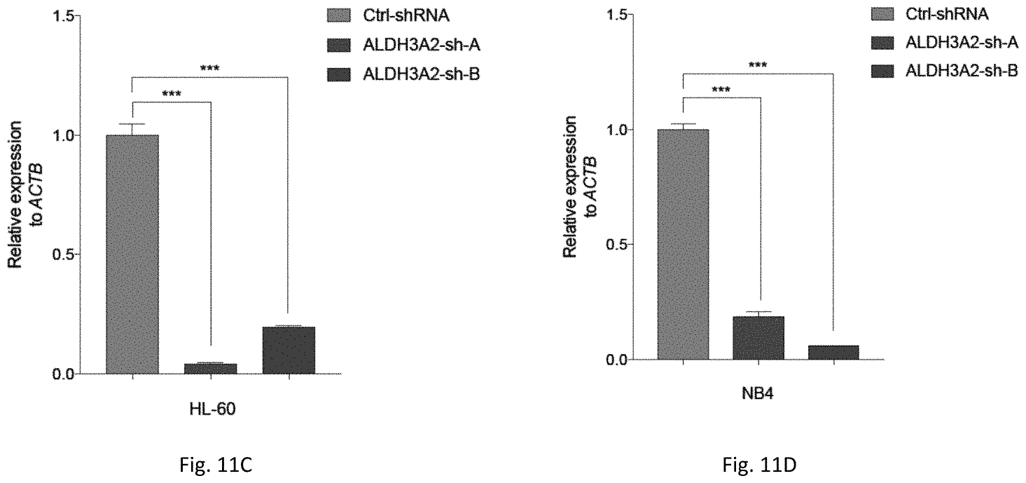

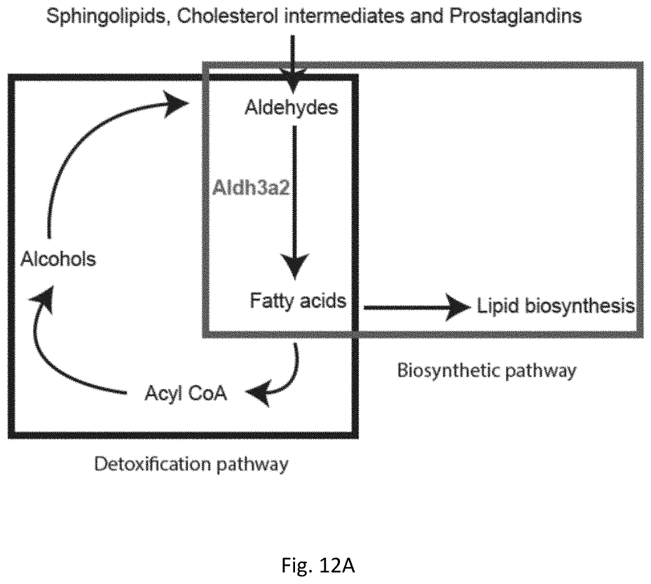

To evaluate the importance of ALDH3A2 in human leukemia five different AML cell lines were utilized with a range of ALDH3A2 expression (FIG. 4C); 3 cell lines with MLL-AF9 translocations (MOLM-14, THP1 and NOMO-1) and two acute promyelocytic cell lines (HL60 and NB4). Two independent shRNAs (ALDH3A2-sh-A and ALDH3A2-sh-B) that efficiently silence ALDH3A2 (FIG. 4D and FIG. 11) were used. All five cell lines were sensitive to ALDH3A2 knockdown (FIG. 4E) regardless of the presence of an MLL-AF9 fusion oncoprotein. In addition, primary human AML samples were also sensitive to ALDH3A2 knockdown (FIG. 4F).

Taken together these results demonstrate that human AML is sensitive to ALDH3A2 deletion and that targeting aldehyde metabolism represents a therapeutic opportunity.

### Example 5-ALDH3A2 Depletion Alters the Redox State of Cells

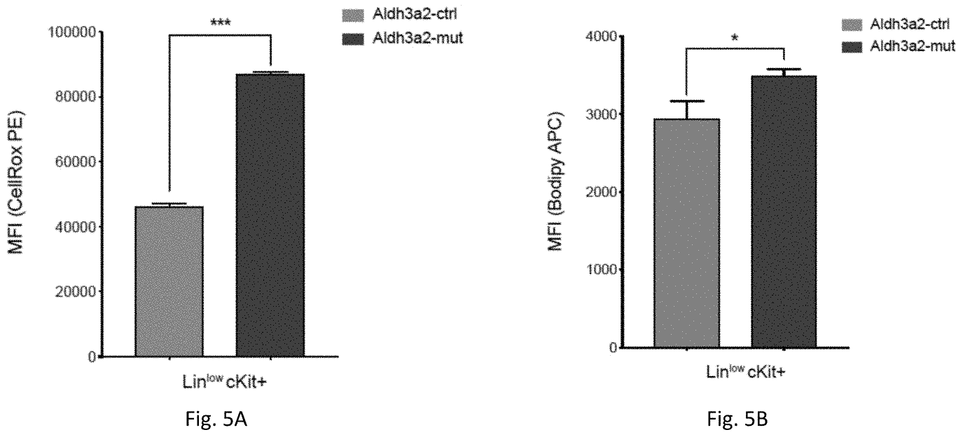

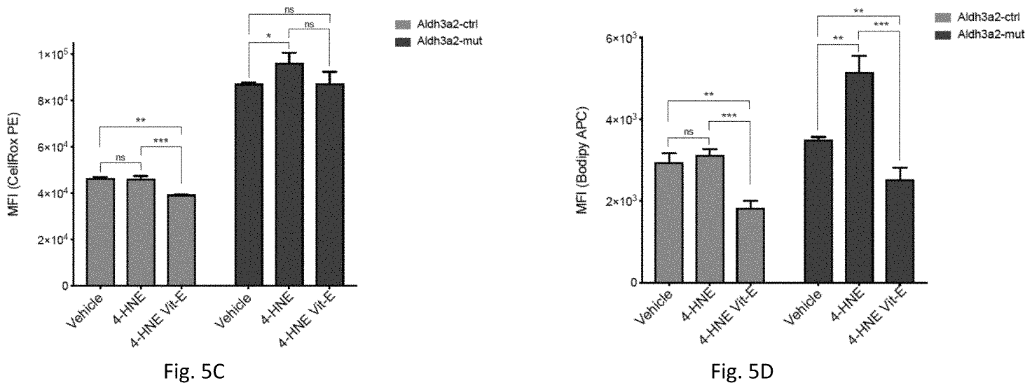

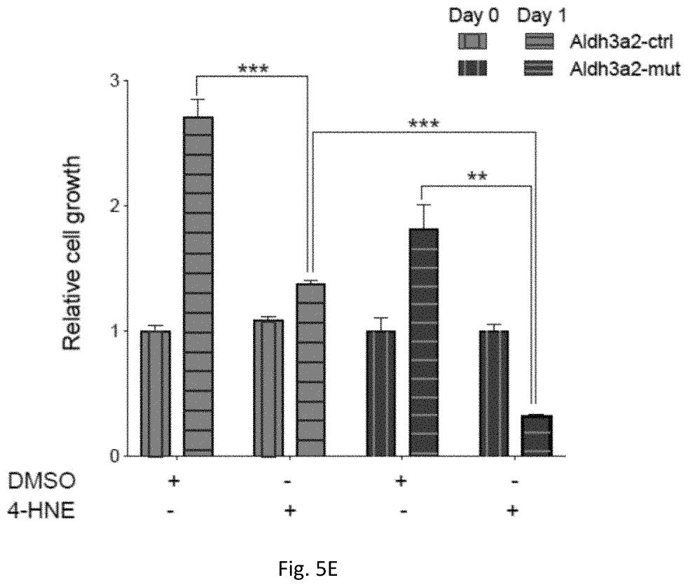

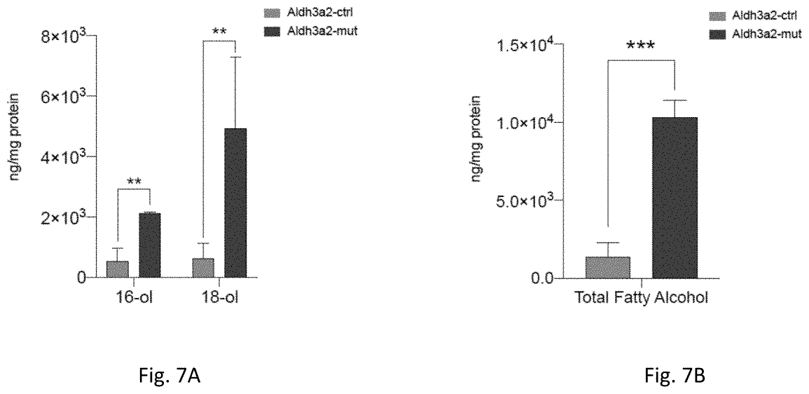

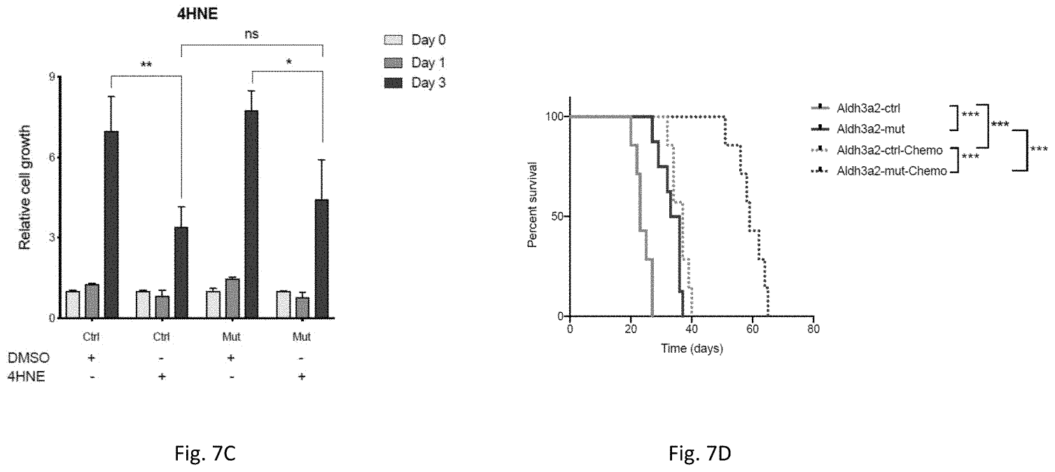

Aldehyde dehydrogenases metabolize aldehydes to mitigate oxidative stress and prevent lipid peroxidation in the cell. Since fatty aldehydes are highly unstable, the levels of fatty alcohols were measured, precursors of fatty aldehydes and shown to accumulate in Aldh3a2 deficient cells Sjögren-Larsson syndrome cells. Indeed, endogenous levels of 16- and 18-carbon fatty alcohols together with total fatty alcohol levels were greatly increased in Aldh3a2 mutant leukemia compared to control leukemia cells (FIG. 7A-B). Next, oxidative damage in AML cells lacking Aldh3a2 was examined. Measurement of cellular reactive oxygen species (ROS) and lipid peroxidation by flow cytometry showed increased levels in mutant versus Aldh3a2-control leukemia cells (FIGS. 5A-5B). To further assess deficits in detoxification capacity, the effects of 4-hydroxynonenal (4-HNE) was studied next, a commonly investigated fatty aldehyde and a byproduct of arachidonic acid catabolism, on cellular ROS, lipid peroxidation and proliferation in Aldh3a2-deficient leukemia stem and progenitor cells. Exposure to 4-HNE for 24 hours increased cellular ROS in mutant but not control cells (FIG. 5C). Vitamin E, a quencher of lipid phase ROS, decreased total ROS in control cells but was unable to do so in mutant cells (FIG. 5C). Likewise, exposure to 4-HNE increased levels of lipid peroxidation in mutant but not in control cells (FIG. 5D). In contrast to its effects on ROS, Vitamin E treatment significantly decreased lipid peroxidation in both populations likely quenching lipid peroxides present at baseline in control cells (FIG. 5D). Finally, although both control and mutant cells show reduced cell growth in response to 4-HNE exposure, sensitivity to 4HNE was significantly greater in mutant than in control cells (FIG. 5E). Lastly, normal granulocyte monocyte precursors (N-GMPs) were investigated. Treatment with 4-HNE both of control and mutant N-GMPs resulted in similar decreases in cell proliferation with the mutant being no more sensitive than the control cells (FIG. 7C).

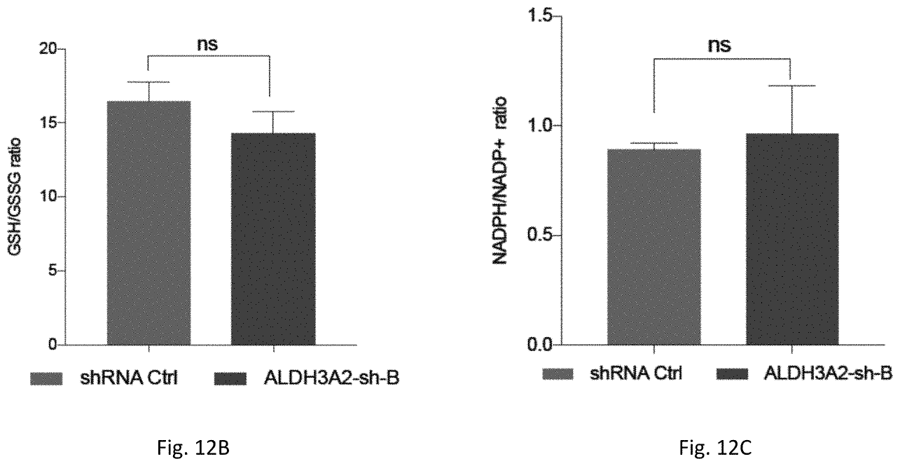

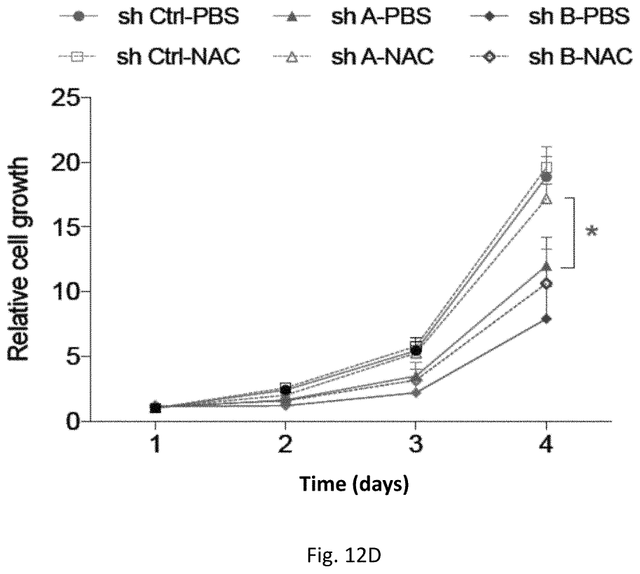

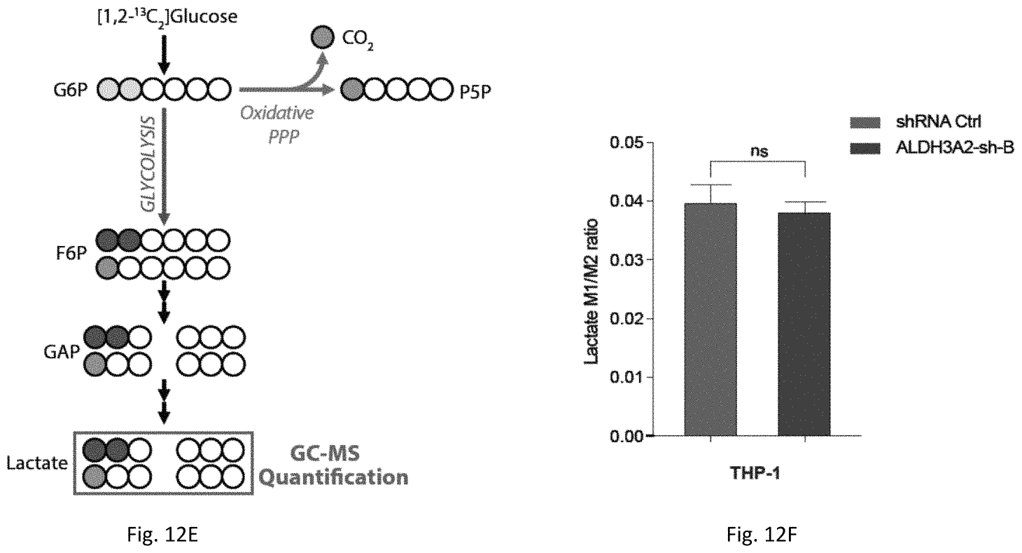

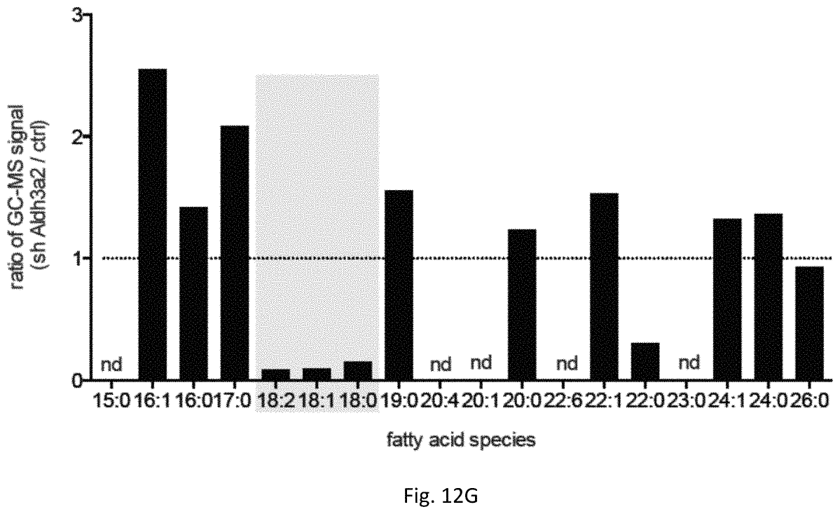

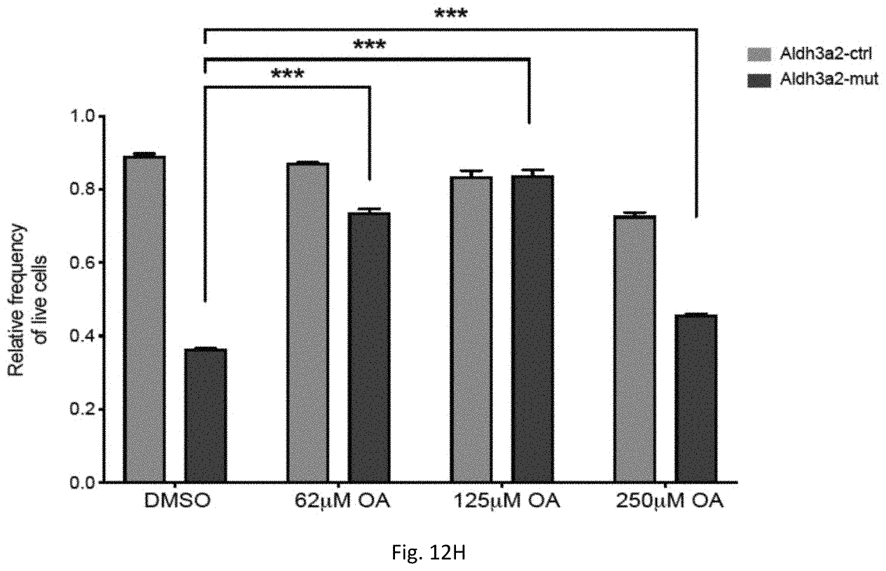

Leukemic cells with knockdown of ALDH3A2 failed to increase common cellular antioxidant defenses, as we did not detect any differences in the reduced/oxidized glutathione ratio or NADPH/NADP⁺ ratio (FIGS. 12B-12C). Rescue of ALDH3A2 depleted THP-1 cells with N-Acetyl Cysteine (NAC) resulted in partial rescue in the population of cells with moderate (sh-A) but not severe (sh-B) ALDH3A2 depletion (FIG. 12D). NAC does not rescue lipid ROS species. Leukemic cells with ALDH3A2 depletion did not increase activity of the oxidative pentose-phosphate pathway (oxPPP), one of the main metabolic pathways generating NADPH. In cells cultured in [1,2-¹³C2]glucose, the oxPPP generates singly ¹³C labeled (M1) intermediates, glycolysis generates doubly 13C labeled (M2) intermediates, and the M1/M2 ratio reflects the oxPPP flux relative to glycolysis (FIG. 12E). However, the M1/M2 ratio was unchanged in THP-1 cells following ALDH3A2 knockdown, suggesting a limited NADPH production response to oxidative stress (FIG. 12F). Cells lacking ALDH3A2 thus fail to increase antioxidant defense mechanisms to mitigate the increased oxidative stress and prevent lipid peroxidation.

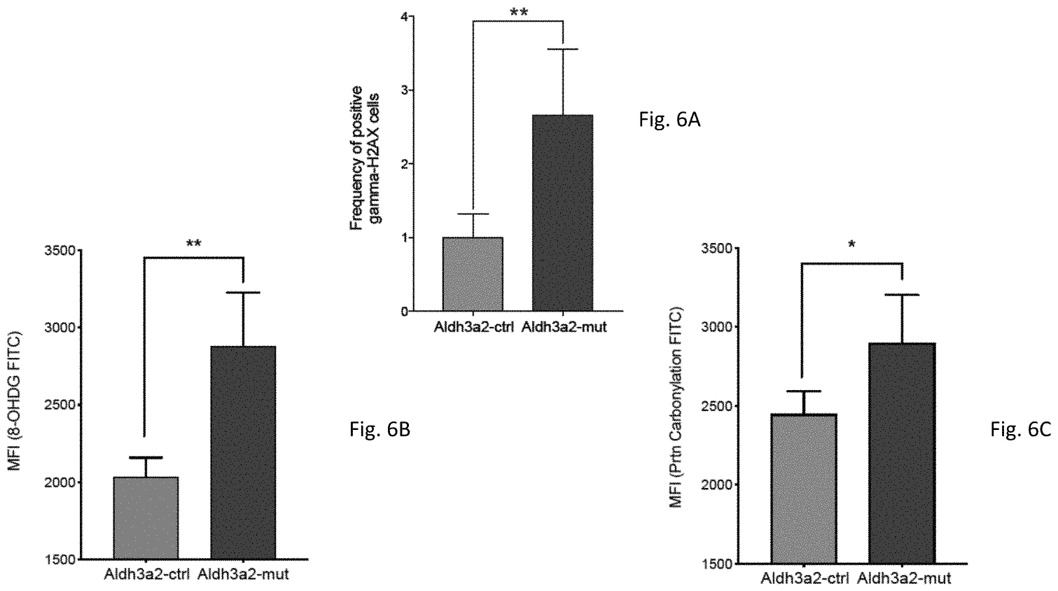

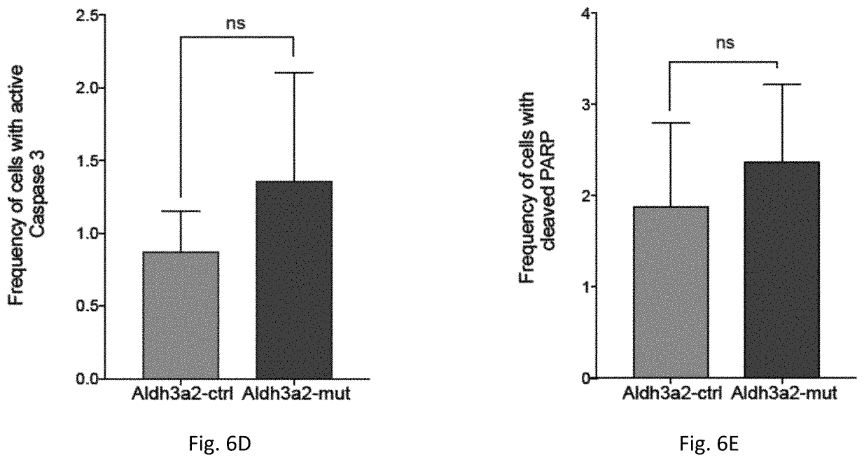

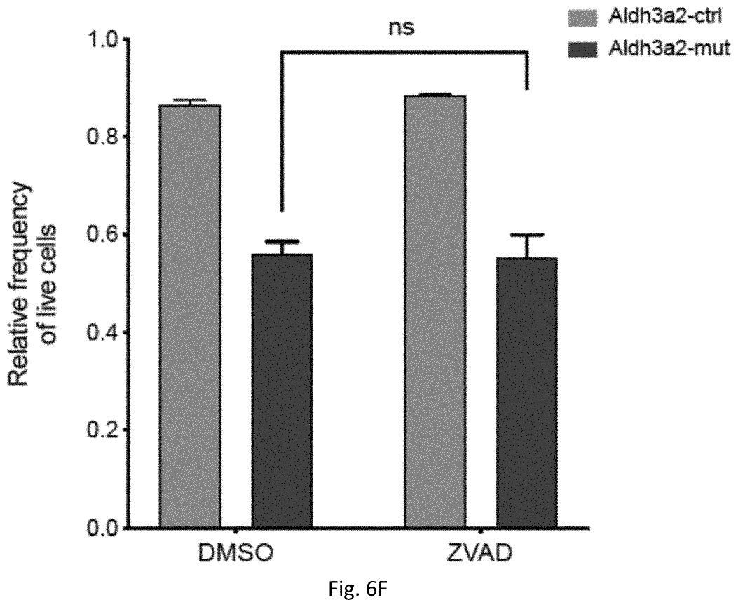

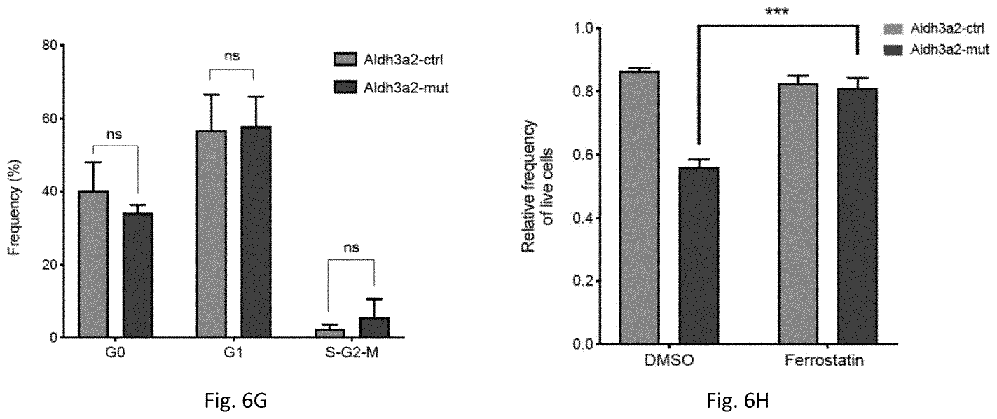

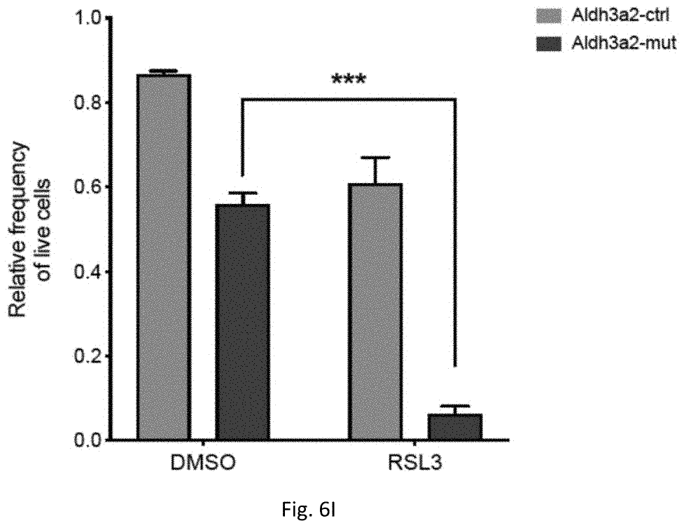

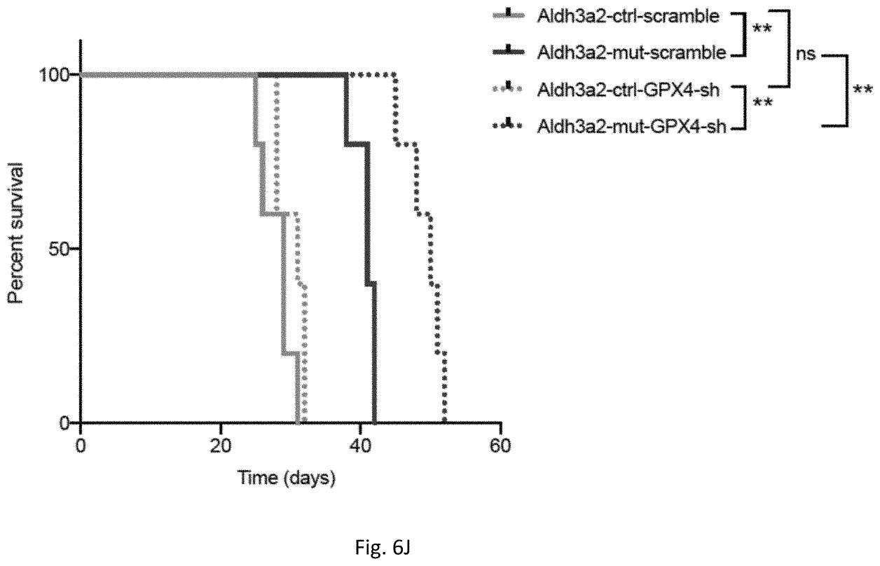

Aldehydes and lipid peroxides are highly reactive and lead to oxidation of other molecules such as DNA and proteins. Oxidative DNA damage, as measured by gamma-H2AX and 8-OHDG levels, was increased in Aldh3a2 mutant compared to control leukemic cells (FIGS. 6A and 6B). Levels of protein carbonylation, representing another consequence of oxidative stress, were also increased in mutant leukemic cells (FIG. 6C).

Taken together, these data show that deficiency of Aldh3a2 in leukemia progenitor cell populations, but not normal hematopoietic progenitor cells, leads to increased oxidative damage.

### Example 6-Loss of Aldh3a2 Impacts Lipid Metabolism

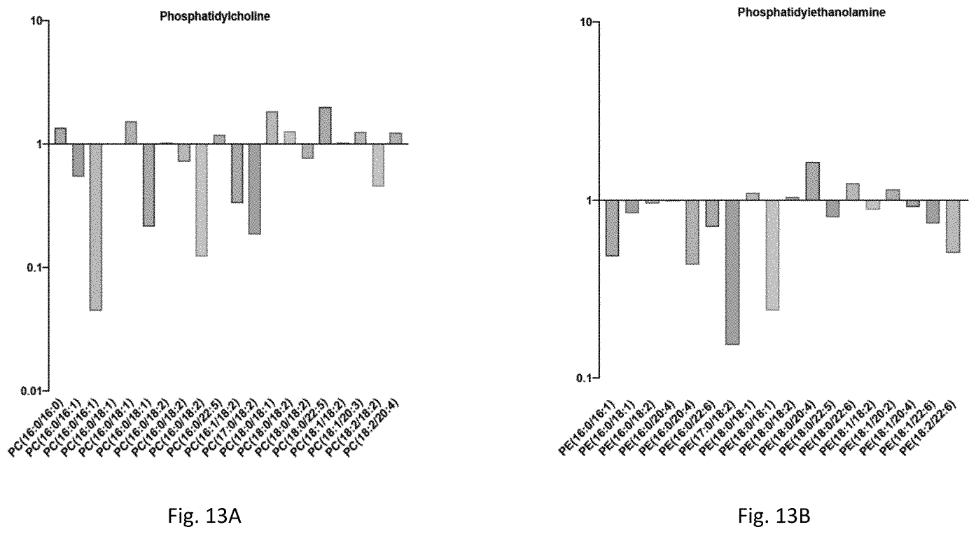

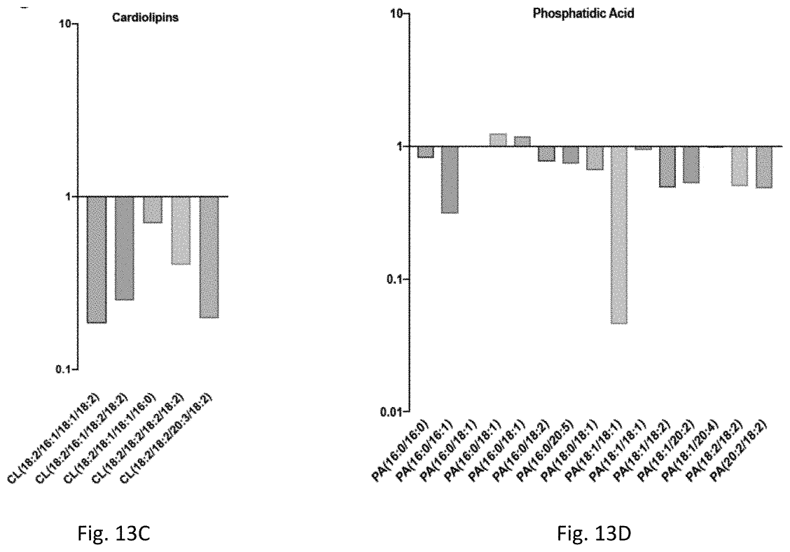

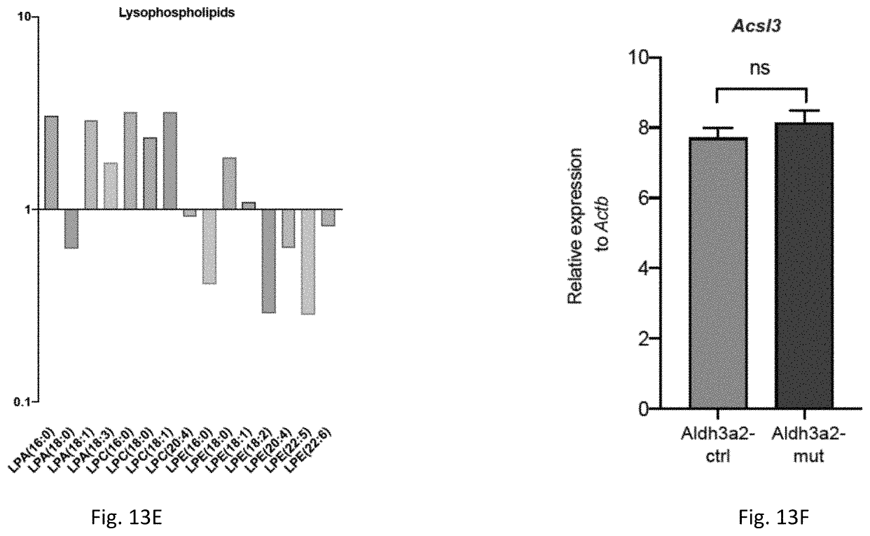

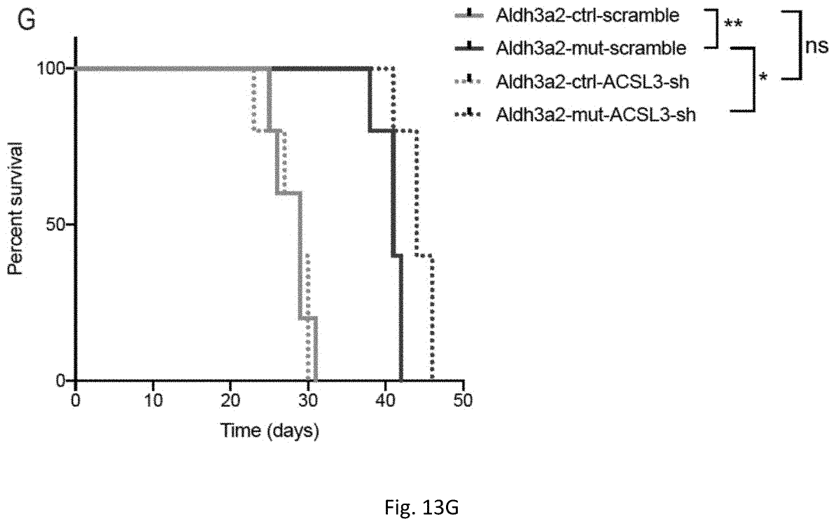

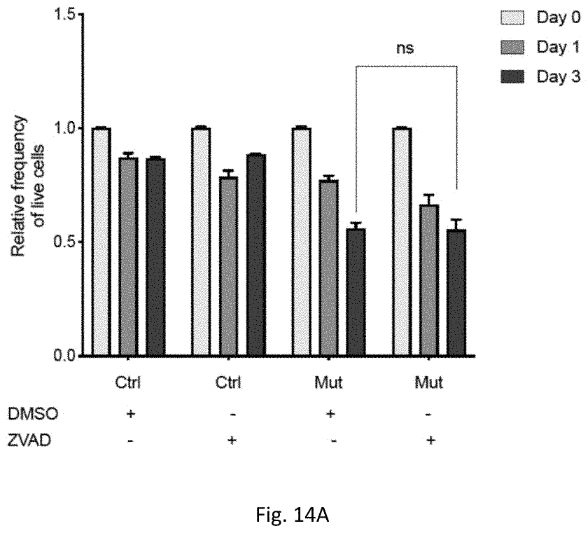

By detoxifying fatty aldehydes, Aldh3a2 generates fatty acids that can be used for biosynthetic purposes (FIG. 12A). Fatty acid species with a length of 18 carbons were strongly decreased in L-GMPs lacking Aldh3a2 (FIG. 12G). Supplying leukemic cells with exogenous oleic acid (18:1) rescued the effects of Aldh3a2 depletion in leukemic cells from conditional mutant mice (FIG. 12H). To further investigate the consequences of this depletion of 18-carbon fatty acids in Aldh3a2 knockout cells lipidomics analysis was performed on leukemic cells derived from conditional knockout mice (FIGS. 13A-13E). Several, but not all, phospholipid species containing 18-carbon fatty acid tails were decreased in mutant cells across lipid classes, including phosphatidylcholines, phosphatidylethanolamines, cardiolipins and phosphatidic acids (FIGS. 13A-13D). Interestingly, changes in several lysophospholipid species were also observed, with an increase in lysophospholipids with a saturated fatty acid (SFA) or monounsaturated fatty acid (MUFA) as remaining tail, but a decrease in lysophospholipids with a long chain polyunsaturated fatty acid (PUFA) tail (FIG. 13E).

These results show that besides increasing oxidative damage, loss of Aldh3a2 also alters biosynthetic pathways and cellular lipid composition in leukemic cells.

### Example 7-Aldh3a2 Depletion Causes Ferroptosis and is Synergistic with GPX4 Inhibition

The specific mechanism for the reduced number of leukemic cells upon Aldh3a2 deletion was next investigated. Cell cycle profiles were similar in leukemic cells from both control and mutant Aldh3a2 mice (FIG. 6G), indicating that the change is likely due to cell death rather than reduced proliferation. Aldh3a2-mutant leukemic cells however did not show activation of caspase-3 (FIG. 6D) nor cleavage of the caspase-3 target PARP (FIG. 6E), which characterize the normal apoptotic cascade. Furthermore, treatment of primary L-GMP-enriched (Lin⁻, cKit⁺) cells with the pan-caspase inhibitor Z-VAD did not rescue the cell death observed in Aldh3a2 deficient cells (FIG. 6F and FIG. 13H), arguing against an apoptotic cell death program in primary leukemic cells.

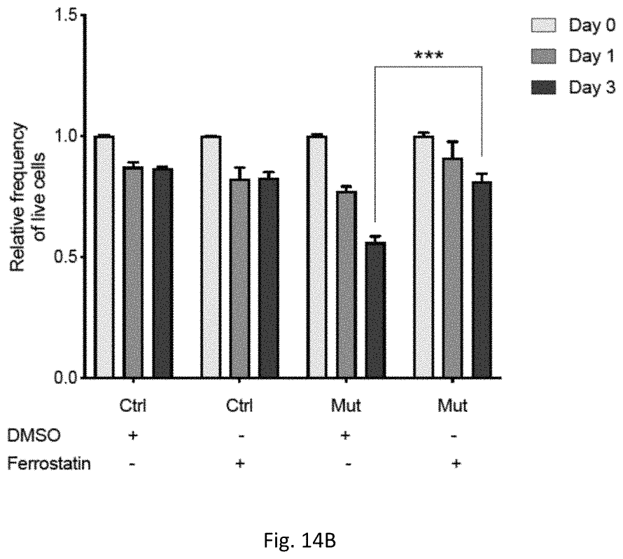

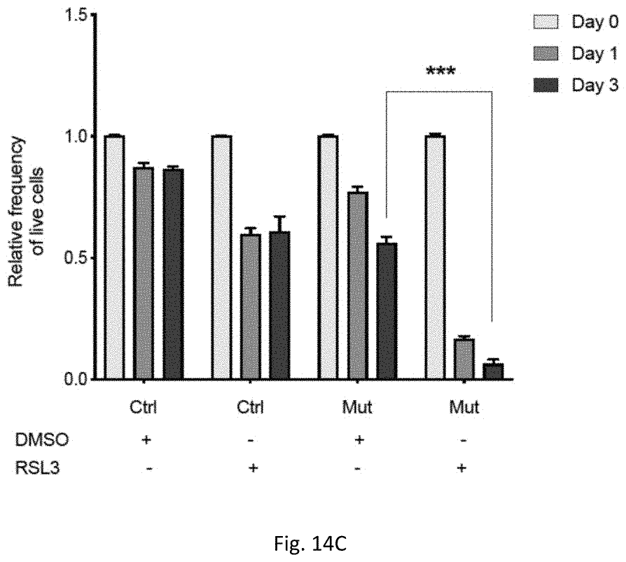

Lipid peroxidation has been linked to the induction of ferroptosis, a non-apoptotic, iron-dependent form of cell death. An increase in lysophospholipids, especially those lacking PUFA tails as was observed (FIG. 13E), is a hallmark of ferroptosis. In addition, the ability to rescue cell death by providing exogenous MUFAs, as was found (FIG. 12H), is another feature of ferroptotic cell death. To further confirm these findings cells were treated with ferrostatin, an inhibitor of iron-dependent oxidative damage. Ferrostatin completely prevented the cell death observed in Aldh3a2 mutant leukemia cells (FIG. 6H and FIG. 14B).

To further assess the relationship to ferroptosis, a key enzyme protecting cells from ferroptotic cell death, glutathione peroxidase 4 (Gpx4) was genetically and pharmacologically targeted. Aldh3a2 deficient Lin⁻cKit⁺ AML cells showed increased sensitivity to the Gpx4-inhibitor RSL3 (FIG. 6I and FIG. 14C). Moreover Gpx4 knockdown significantly prolonged survival in animals receiving Aldh3a2-mutant cells but not control cells (FIG. 6J), demonstrating a superadditive effect of Aldh3a2 deficiency and ferroptosis activation by Gpx4 inhibition. Recent findings have shown that activation of MUFAs by Acs13 can also promote resistance to ferroptosis. Acs13 expression was not different between Aldh3a2 control and mutant leukemic cells (FIG. 13F). Acs13 knockdown modestly prolonged survival in animals receiving Aldh3a2-mutant cells (FIG. 13G), but this effect was much less pronounced than that of Gpx4 depletion.

Thus, the results show that loss of Aldh3a2 induces ferroptotic cell death in leukemic cells, and is synthetically lethal with Gpx4 inhibition.

### Example 8- Therapeutic Implications of Aldh3a2 Depletion

Lastly, in order to test the therapeutic potential of Aldh3a2 depletion in combination with conventional chemotherapy, Aldh3a2 control and mutant leukemic mice were given cytarabine and doxorubicin. Mice receiving either chemotherapy or Aldh3a2 depletion survived longer than those receiving placebo. This prolongation was much more pronounced when chemotherapy and Aldh3a2 depletion was combined. Therefore, combining Aldh3a2 inhibition and standard cytotoxic chemotherapy may have therapeutic implications (FIG. 7D).

### Discussion

To identify pathways critical for leukemia maintenance, an shRNA screen was designed that focused on regulators of metabolism. Aldh3a2, an enzyme responsible for detoxification of fatty aldehydes and generation of C16-18 fatty acids, was identified as a specific metabolic vulnerability of leukemic stem and progenitor cells.

Fatty aldehydes accumulate in cells as a result of alcohol metabolism, pyrimidine and purine synthesis, amino acid metabolism and lipid peroxidation. Aldehydes are highly reactive compounds that can damage, DNA, proteins and lipids. Aldh3a2 is a metabolic enzyme responsible for detoxification of fatty aldehyde, especially those with a medium to long carbon chain (primary substrates are thought to be fatty aldehydes of 16 or 18 carbon length), resulting in the generation of C16-18 fatty acids.

It is shown here that Aldh3a2 is critical in protecting leukemic stem and progenitor cells from ferroptotic cell death. Ferroptosis is a non-apoptotic, iron-dependent form of cell death that is driven by excessive lipid peroxidation. Because AML and leukemic stem cells have higher iron requirements and oxidative metabolism than normal cells, they are more vulnerable to ferroptosis induction. In accordance, it was observed that loss of Aldh3a2 increases oxidative stress and lipid peroxidation in AML cells, leading to ferroptotic cell death. In contrast, it was found that normal murine stem and progenitor cells are tolerant of the loss of Aldh3a2. In addition to exhibiting a normal oxidative metabolism, non-cancer GMPs express lower levels of Aldh3a2 than AML cells. Thus, loss of Aldh3a2 in normal hematopoietic stem and progenitor cells likely does not reach the critical threshold of oxidative damage required for ferroptosis induction.

Increasing oxidative stress by inhibiting the glutathione peroxidase Gpx4 further augments the damaging, death-inducing effects of Aldh3a2 depletion. While both Aldh3a2 and Gpx4 depletion can be rescued by ferrostatin, they have distinctive functions acting respectively on aldehydes and peroxides. Since aldehydes and peroxides can contribute to formation of the other, it is reasonable to view Aldh3a2 and Gpx4 as serving in parallel, detoxifying oxidative or peroxidative products that result from an increased or disregulated metabolic activity in malignancy. In AML, inhibition of Gpx4 alone had limited ability to enhance cell death. Yet, when inhibiting both Aldh3a2 and GPX4, a synthesis of effect is evident with superadditive increased cell killing, an oxidative product synthetic lethality.

Further distinguishing Aldh3a2 from Gpx4 is its participation in fatty acid synthesis. By contributing to the de novo generation of long chain fatty acids, Aldh3a2 can contribute to both the replenishment of damaged fatty acids and limiting the damage of existing fatty acids through its enzymatic activity. In contrast, Gpx4 acts only on peroxide damaged lipids. These distinctions may contribute to the greater potency of Aldh3a2 compared with Gpx4 inhibition on AML.

Therapeutic index is a critical feature in considering a target for drug development and it was found that normal murine stem and progenitor cells are tolerant of the loss of Aldh3a2. The enzyme is expressed in N-GMPs though at a lower level than L-GMPs, potentially accounting for leukemia's greater dependency on it. A greater dependency is also anticipated in AML given the well established upregulation of oxidative metabolism in AML and leukemic stem cells. Targeting Aldh3a2 may provide a distinctive means of selectively killing malignant over normal hematopoietic cells, particularly primitive L-GMPs, and merits consideration in therapeutic development.

Depletion of Aldh3a2 has been thought to result in cell death by causing an increase in toxic C16-C18 aldehydes and alcohols and incomplete lipid metabolism in the inherited pediatric Sjögren-Larsson Syndrome. Sjögren-Larsson Syndrome is an autosomal recessive disease characterized by ichthyosis, neurodevelopmental delay and photophobia. Genetic carriers are not affected and the disease in homozygotes generally does not progress beyond childhood. These features suggest that targeting Aldh3a2 may be tolerated in extra-hematopoietic tissues at least for intervals that would be anticipated in anti-leukemic therapy.

The target (Aldh3a2) has been identified and validated through genetic approaches in mouse and human AML cell lines, both in vitro and in mouse models in vivo. The next steps may include assay development and validation for high-throughput screening (HTS) to identify a small molecule inhibitor of Aldh3a2.

The target patient population may include newly diagnosed AML patients but will be first tested in patients that progressed on standard of care treatment. A rapid screening assay may be developed that would allow identification of patients that will most likely respond to an Aldh3a2 inhibitor therapy prior to enrollment. In brief, patient cells will be incubated overnight with the Aldh3a2 inhibitor and dilutions of lipid peroxidation inducers, after which the level of lipid peroxidation is measured fluorometrically to assess treatment response.

It is proposed that targeting this locked-in metabolic addiction can show efficacy as a single agent, or sensitize the cancer cells to standard chemotherapy. In preclinical mouse models of AML, it was found that synergistic elimination of AML cells by combining Aldh3a2 deletion with a standard chemotherapy regimen. Combination with targeted therapies may also be explored. The kinase inhibitor Sorafenib, which is effective in inducing remission in AML patients that carry the Flt3 mutation, is reported to induce ferroptosis. Using mouse models that carry the Flt3-ITD mutation, the potential of combining Aldh3a2 inhibition with Sorafenib may be explored.

Preliminary studies highlight the potential of targeting Aldh3a2 for AML therapy, either alone or in specific combinations. While Aldh3a2 is dispensable for normal HSCs and GMPs by the in vivo knock-out studies, other members of the ALDH family play important roles in normal hematopoiesis. An inhibitor specific to Aldh3a2 would thus be ideal with regards to therapeutic development. High-throughput screening (HTS) may be performed to identify small molecule inhibitors of Aldh3a2, and validate and optimize hits with the goal of developing a potent and selective inhibitor of Aldh3a2 that can be carried to further (pre)clinical testing.

There is currently no approved therapy for cancer targeting Aldh3a2 or inducing lipid peroxidation in other ways. Targeting GPX4 is being explored preclinically for cancer therapy, but results indicate that this is not a viable target in AML. It is proposed here that targeting Aldh3a2 will have broader effects than the targeting of GPX4 in AML and in cancer in general, with possible synergism through combined inhibition of Aldh3a2 and GPX4.

In summary, Aldh3a2 depletion results in an iron-dependent oxidative cell death of leukemia cells while sparing normal hematopoiesis. The combination of Aldh3a2 inhibition with ferroptosis inducers or with standard AML induction chemotherapy deserves further consideration as a cancer therapy.

### Reagent and Assay Development and Optimization

In future work, a set of assays will be developed for primary and secondary screening of Aldh3a2 inhibitors. For primary HTS, a biochemical enzyme activity assay will be developed, and for secondary screening, several cell based-assays will be optimized.

First, this includes a HTS-compatible primary screening assay. An Aldh3a2 biochemical assay will be optimized. Aldh3a2 activity is assayed fluorometrically by measuring the fatty aldehyde-dependent production of NADH. The reaction mixture consists of a 50 mM Tris-HCl buffer (pH 8.5) with 150 mM NaCl, 10% glycerol, 0.1% Triton X-100, 500 μM NAD and 100 μM fatty aldehyde (hexadecanal or octadecanal). As enzyme, the use of either affinity-purified Aldh3a2 protein (5-10 ng for a 25 μl reaction) or cell lysate obtained from a human AML cell line (0.2-2 μg for a 25 μl reaction will be tested; obtained from THP-1 cell line or similar). The reaction is incubated at 37° C. and monitored by measuring the fluorescence of the NADH product (excitation at 356 nm and emission at 460 nm), or using bioluminescence after NADH conversion (NADH-GLO assay). If needed, the reaction can be stopped by the addition of p-chloromercuribenzoate or by changing the pH. The substrate type and amount, enzyme source and amount, the reaction incubation time, reaction stopping conditions and read-out will be optimized. Disulfiram, a pan-ALDH inhibitor, will be used as positive control to calculate the Z′ score. A Z′ score of >0.5 is desired to proceed to HTS.

Second, cell-based secondary screening assays will be performed. Several cell-based secondary screening assays will be developed, that use ALDH activity, lipid peroxidation and/or cell viability as readouts. (i) Using CRISPR/Cas9 an Aldh3a2-knockout BJ human fibroblast cell line will be created. The ALDEFLUOR™ reagent will be used to measure ALDH activity (flow cytometry). This assay will allow to test for compounds that decrease ALDH activity in wildtype but not Aldh3a2-knockout BJ cells. Addition of 4-HNE may be used to increase sensitivity. (ii) Using CRISPR/Cas9 an Aldh3a2-knockout persister human AML cell line (THP-1 cell line or similar) will be created. The ALDEFLUOR™ reagent will be used to measure ALDH activity (flow cytometry) and BODIPY™ 581/591 C11 reagent will be used to measure lipid peroxidation (flow cytometry). This assay will allow to test for compounds that decrease ALDH activity or increase lipid peroxidation in wildtype but not Aldh3a2-knockout AML cells. Addition of 4-HNE may be used to increase sensitivity. (iii) Sensitization of a human AML cell line (THP-1 cell line or similar) to dilutions of the GPX4 inhibitor RSL3 will be analyzed. Measurement of cellular ATP levels (cellTiter-GLO assay) will be used as a read-out of cell viability (bioluminescence). This assay will allow to test for compounds that sensitize human AML cells to GPX4 inhibition. Addition of 4-HNE may be used to increase sensitivity.

### Hit Generation

### Primary HTS:

In future work, after validation of the biochemical assay, HTS will be performed to identify small molecule inhibitors of Aldh3a2. The initial screen will be performed on the ChemDiv 6 library, a select set of 44,000 public domain compounds. This fully annotated, high quality collection was selected by drug discovery experts to provide drug-like qualities, chemical diversity, and potential for structure-activity relationship (SAR) follow-up experiments. Compounds that are 3 Z-scores away from the average of all compounds will be considered hits.

### Confirmation & Validation of Hits:

This future work will involve (i) re-testing of hits in the primary screening assay(s): depending on the assay used for the primary HTS, either or both the affinity-purified protein-based and cell lysate-based assays will be used to re-test hits and confirm their activity. (ii) Generation of dose-response curves: hits that pass re-testing will used to generate dose-response curves. Only compounds with activity in the nM to low μM range will be carried to the hit-to-lead phase.

### Hit-to-Lead

### Characterization of Hits:

This future work will involve (i) confirm key structure of hits, (ii) affinity for target (Ki), inhibitory activity (enzymatic IC₅₀), (iii) cellular permeability using PAMPA assay, solubility and metabolic stability, and (iv) early ADME assays: e.g. hERG inhibition, CYP inhibition/induction, glutathione trapping.

### Secondary Screening Assays:

Validated hits will next undergo secondary screening in live cell-based assays to determine potency. The use of Aldh3a2 knock-out versus wildtype cells in several of the described assays will allow to determining specificity and off-target activity of the hits.

Test for selectivity: Further testing for selectivity against related ALDH enzymes will be performed using biochemical assays.
Structure optimization: Analogous compounds will be tested to determine a structure-activity relationship (SAR), with re-iterative compound characterization. This will include computer modeling and crystallography/structure-based design.
Pharmacokinetics (PK) discovery: A first round of in vivo testing (mouse/rat) will be performed in order to select compounds with reasonable in vivo PK properties.
Pharmacodynamics (PD) discovery: In vivo target engagement biomarkers (mouse/rat) will be evaluated in order to select compounds with reasonable in vivo PD properties. Using mass spectrometry, cellular read-outs of target engagement both in vitro and in vivo (using blood plasma or circulating AML cells as input) will be defined.

### Future Lead Optimization

Optimization of molecular and cellular properties: IC₅₀, subtype selectivity, SAR information.
Optimization of ADME properties: lipophilicity, aqueous solubility, hepatic microsome stability, hERG inhibition, CYP inhibition/induction, glutathione trapping, plasma stability, plasma protein binding, hepatotoxicity and gastrointestinal tract permeability.
In vivo studies: Different in vivo studies will be performed in mouse and/or rat to demonstrate a therapeutic index. These will include in vivo PK/PD, efficacy, tolerability and toxicology studies. (i) Mouse models: a. mouse AML models: MLL-AF9 (retroviral and transgenic; both available), HoxA9-Meis 1 (retroviral; available), MLL-ENL (retroviral; in development), AML1-ETO (retroviral; in development), Tet2/Flt3-ITD (transgenic; in development), NPM1/Dnmt3a (transgenic; in development). All lines are labeled with a fluorescent protein (GFP, mTomato) and/or luciferase and are engrafted (i.v. injection) in wildtype recipient mice, allowing easy disease monitoring. b. Human AML patient-derived xenografts (PDXs) or cell line xenografts: AML PDXs include pediatric and adult primary AML cells engrafted in NSG mice (2 lines available; 4 additional lines in development), 4 AML cell line xenografts models are available (THP-1, NOMO1, MONOMAC6, MOLM-14 cell lines engrafted in NSG mice). (ii) Read-outs: PK/PD studies, AML cell elimination, mouse survival, expected cellular effects (lipid peroxidation, DNA damage), synthetic lethality with GPX4 inhibition or induction chemotherapy.
Other cancer types: The activity of the most advanced molecules on other cancer types (in cell culture and/or mouse models) will be evaluated to identify other potential cancer patient populations that could benefit from an Aldh3a2 inhibitor therapy. The focus will be on cancers that are known to have a dysregulated oxidative metabolism, including other blood cancers and solid tumors harboring K-Ras, B-Raf mutations or Myc activation. Screening of lead compounds on the NCI60 cancer cell line encyclopedia can be used to identify other tumor types of interest.
Development of an overnight ALDH3A2 inhibitor sensitivity assay: An overnight assay will be developed to select patients that would respond to an Aldh3a2 inhibitor. For assay optimization cells from human PDXs and mouse AML models will be used, allowing correlation between response in the overnight assay and in vivo response. Cells will be cultured for 16 hours in the presence of dilutions of the lead ALDH3A2 inhibitor, either alone or together with the fatty aldehyde 4-HNE or the GPX4 inhibitor RSL3. Lipid peroxidation will be used as read-out (flow cytometry or fluorometry). Next, AML patient samples will be obtained (>300 different AML patient samples available). Response will be correlated in the overnight assay to patient properties, including mutational background and prior treatment.

### Materials and Methods

### Mice

C57BL/6J, B6.SJL-Ptprca Pep3b/BoyJ (abbreviated as B6.SJL), B6.Cg-Tg[Mx1-cre] 1Cgn/J (abbreviated as Mx1-Cre), C57BL/6-Tg(CAG-EGFP)1Osb/J mice (abbreviated as actin-GFP), B6.Cg-Tg(CAG-DsRed*MST)1Nagy/J (abbreviated as actin-DsRed mice) and B6.129S4-Gt(ROSA)26Sor^{tml(FLP1)Dym}/RainJ (abbreviated as FLP recombinase mice) were purchased from Jackson Laboratory. Frozen embryos for C57BL/6NTac-Aldh3a2^{tmla(EUCOMM)Wtsi}/IcsOrl were obtained from EMMA mouse repository and grown into adult mice. Constitutional Aldh3a2 deficient mice (on a C57BL/6J background) were made and obtained from the laboratory of Dr. William Rizzo at University of Nebraska Medical Center.

For the development of the Aldh3a2 conditional knockout model, C57BL/6NTac-Aldh3a2^{tmla(EUCOMM)Wtsi}/ICsOrl mice were crossed with FLP recombinase-expressing mice. The mice thus obtained designated Aldh3a2 loxp/loxp mice were crossed with Mx1-Cre mice to obtain Aldh3a2 loxp/loxp; MxCre1 −/− (abbreviated Aldh3a2-Ctrl mice) or Aldh3a2 −/−; MxCre1 +/− mice (Abbreviated Aldh3a2-mut mice). The Harvard University Institutional Animal Care and Use Committee and the Subcommittee on Research Animal Care of the Massachusetts General Hospital approved all animal work.

### Screen

The screen was carried out in 384 well plates arrayed with pLKO.1 based lentiviruses expressing shRNAs against the genes selected. Control wells contained lentiviruses expressing empty pLKO.1 vector. Eight hundred Ds-Red positive L-GMPs or N-GMPs were plated into wells. Polybrene (Millipore Sigma) and spinfection was used for infection and puromycin (Invivogen) was used for selection of infected cells.

After 36 hours of selection, 2200 GFP positive stroma cells were added to the wells in conditioned media. Conditioned media is media in which the fourth passage of CD45- and CD105+ positive cells expressing GFP under a beta-actin promoter have been maintained for four days prior. This co-culture proceeded for 96 hours and the plates were then imaged using an Image Xpress Microscope (Molecular Devices, Sunnyvale, Calif.). Data was analyzed using MetaXPress Software (Molecular Devices) and CellProfiler.

Schema for scoring of hits is defined in FIG. 8.

### Survival Analysis

Six- to 12-week-old Aldh-mut and Aldh-Ctrl mice were used to generate MLL-AF9 leukemia through retroviral transduction. This was transplanted into lethally irradiated (9 Gy) “primary leukemic” C57BL/6J mice and then into sub-lethally irradiated (4.5 Gy) “secondary leukemic” C57BL/6J mice (4.5Gy). Forty-eight hours after injection into secondary recipients these mice received three doses of Polylnosinic-PolyCytidylic acid (Poly(I).Poly(C)) (GE Healthcare) on alternate days. These mice were followed out to moribund status.

### Characterization of Normal Hematopoiesis

Constitutional as well as conditional Aldh3a2 mice were utilized. Enumeration of normal cell types and transplant assays were performed as previously described.

### Measurement of Aldh3a2 Enzyme Activity

FALDH was assayed in homogenates of 0.5 million mononuclear bone marrow cells from Aldh3a2 WT and Aldh3a2 KO mice using octadecanol (Sigma) as substrate as previously described.

### Measurement of Alcohols

Alcohols were measured in bulk leukemic cells as described.

### Lipidomics

Sorted GFP⁺ leukemic cells obtained from the bone marrow of Aldh3a2 WT and Aldh3a2 KO mice were lysed in ice-cold methanol and non-polar metabolites were extracted using methanol-chloroform phase separation (methanol:water:chloroform in 2:1:4 ratio). Lipidomic profiling was then performed on the chloroform fractions on a ThermoFisher Q-exactive equipped with HESI source and BioBond column (50×4.6 mm, 5 μm C4, with guard column; Dikmatech). A volume of 15 μl was injected and the full mass spectrum was obtained in both positive and negative mode (0 to 60 minutes, resolution 70,000, AGC target 1e6, m/z range 150 to 2000; dd-MS2: resolution 35,000, AGC target 1e5). For positive mode mobile phase A consisted of 5 mM ammonium formate, 0.1% formic acid, 5% methanol, in water and mobile phase B of 5 mM ammonium formate, 0.1% formic acid, 5% water, 35% methanol, 60% isopropanol. For negative mode mobile phase A consisted of 0.03% ammonium hydroxide, 5% methanol, in water and mobile phase B of 0.03% ammonium hydroxide, 5% water, 35% methanol, 60% isopropanol. Analysis was performed in an untargeted manner using the LipidSearch software (Thermo Fisher Scientific).

### Lipid Peroxidation Assay

Spleens from Aldh3a2-Ctrl and Aldh3a2-mut mice were utilized to obtain single cell suspensions and were incubated with C11 Bodipy lipid peroxidation sensor (ThermoFisher Cat #C10445) or CellRox reagent (ThermoFisher Cat #C10491) for 30 minutes at 370 C. Cells were stained with biotinylated antibodies against lineage markers, SA-Pacific Orange conjugate (ThermoFisher) and APC Rat Anti-Mouse CD117 (BD Biosciences) and read on the LSRII flow cytometer. Lipid peroxidation and cellular ROS was assessed in Lineage-, c-Kit⁺ and Lineage⁻, c-Kit^{hi} populations.

### Human Cell Line Experiments

AML cell lines, HL60, MOLM-14, MONOMAC-6, NB4, NOMO-1, THP1 were obtained from the American Type Culture Collection (ATCC) and the Leibniz Institute DSMZ-German Collection of Microorganisms and Cell Cultures (DSMZ). Primary human AML Samples were obtained from the University of Pennsylvania Tumor Tissue and Biospecimen Bank and the Massachusetts General Hospital Leukemia Bank. Cells were infected with plko.1 based lentivirus expressing Aldh3a2 shRNA A or B or control shRNA, selected with puromycin for infection and plated in white polystyrene 96 well plates. Celltiter GloR 2.0 reagent (Promega) was reconstituted per manufacturer's instructions and 50 ul was added to each well. The plate was imaged using Gen5 software (BioTek) on the BioTek Synergy HTX Machine (BioTek).

### Drug Experiments

RSL3 (at 5 uM), 4-HNE (5 uM) and ZVAD (at 10 uM) was added to Lin-Kit- or Lin-Kit+ leukemic cells in RPMI containing 10 ng/ul of murine IL3, IL6 and SCF. Ferrostatin (10 uM) was added to bulk leukemic cells in RPMI containing 10 ng/ul of murine IL3, TPO and FLT3L. Number of cells were measured by Celltiter GloR 2.0 assay at days 0, 1 and 3. 10 mM of NAC was added to RPMI with 10 percent FBS and 1 percent P/S for relevant rescue experiments. Drugs were purchased from Sigma.

### Statistics

Unpaired, 2-tailed Student t-test was used. Data have been plotted as average +/−standard deviation (SD) for samples following a normal (Gaussian) distribution. Alternatively, Mann-Whitney U test was used and data have been plotted as median==/−interquartile range. The Kaplan Meyer statistic was utilized for the comparison of survival curves. 5-7 mice were utilized in each group in each experiment. Experiments were repeated at least twice. For analysis of luminescence in 96 well plates, plate maps were created, the average of the luminescence was calculated for each set of wells carrying cells infected with a particular virus. This average was normalized to the average of the luminescence for wells carrying cells infected with control shRNA per day. Plates were read on days 0, 1, 2 and 4 and normalized to day 0 values. Statistical significance is indicated as follows: *P, 0.05, **P, 0.01, and ***P, 0.001.

### Generation of N-GMPs, L-GMPs and Murine Bone Marrow Stroma

### L-GMPS

In order to generate L-GMPs six to eight-week old male mice carrying the DsRed florescent protein under the beta-actin promoter, henceforth referred to as DsRed mice, were treated with 5-flourouracil (5-FU) (Millipore Sigma). Single cell suspension of red blood cell lysed bone marrow was infected with MSCV based retrovirus carrying the MLL-AF9 oncogene in tandem with a neomycin resistance gene (gift from Dr. Scott Armstrong) using polybrene (Millipore Sigma). After two rounds of infection, the infected cells were transplanted into lethally irradiated (9 Grays/Gy of irradiation from a ¹³⁷Cesium gamma source) C57BL/6J mice. These recipient mice designated “primary leukemic mice” became moribund at 8-12 weeks after transplant and were euthanized at the first signs of illness. Mononuclear bone marrow cells from these mice were transplanted into sub-lethally irradiated (4.5 Gy) C57BL/6J mice. These recipients, designated “secondary leukemic mice”, became moribund at four weeks and were sacrificed at the first signs of illness. Mononuclear bone marrow from secondary leukemic mice was subjected to flow sorting in order to obtain DsRed positive L-GMPs according to the scheme of Kristov et al.

### NGMPs

N-GMPs were flow sorted from red blood cell lysed bone marrow from Ds-Red mice as described.

### Primary Murine Bone Marrow Stroma

Each batch of stroma was made from four actin-GFP mice. Mononuclear whole bone marrow was plated in alpha-MEM (Gibco) with 20 percent Fetal Bovine Serum (FBS) (Gibco) and 1 percent Penicillin Streptomycin (P/S) (ThermoFisher Scientific) for 28 days (media changes after 2, 14 and 28 days). Cells were selected for CD105+ cells using biotinylated CD105 antibody and Streptavidin magnetic beads (Dynabeads Biotin Binder, ThermoFisher Scientific) and columns and passaged four times. This fourth passage was maintained in media for 4 days and these cells and media was used for all experiments. The media in which these cells were maintained is referred to as “conditioned media”.

### Production of Retroviruses and Lentiviruses

The MLL-AF9 construct in an MSCV-neomycin vector was a gift from Dr. Scott Armstrong and retroviral production was carried out in HEK293-T cells (American Type Tissue Collection or ATCC) using the pCL-Eco plasmid (Addgene) and Fugene HD transfection reagent (Promega) according to the manufacturer's instructions.

Lentiviruses were made according to The Broad Institute protocol for shRNA/sgRNA/ORF low throughput viral production (10 cm dish/6 well plate) found here: portals.broadinstitute.org/gpp/public/resources/protocols.

### Infection of L-GMPs and N-GMPs

Bone marrow mononuclear cells from 5 secondary leukemia mice (for L-GMPs) or 10 DsRed mice (for N-GMPs) were plated in transfection media which contained 10 ng/ml of Stem Cell Factor (SCF, Peprotech) 10 ng/ml of Interleukin 6 (IL-6, Peprotech) and 6 ng/ml of Interleukin 3 (IL-3, Peprotech) and infected with retrovirus using polybrene and spinfection (X2, 24 hours apart). Cells were harvested 3 hours after spinfection.

### RT-QPCR

RNA was extracted from cells using the RNeasy Micro Kit (Qiagen) and RT-PCR was performed using the SuperScript™ IV One-Step RT-PCR System according to the manufacturer's instructions. Taqman probes were used to perform Q-PCR for murine and human Aldh3a2.

### Flow Cytometry

The cocktail of antibodies staining for lineage antigens for N-GMPs consisted of Biotin labelled anti-mouse antibodies against, CD3e, CD4, CD8, CD19, B220, Gr-1, Mac-1 (from BD Biosciences) and CD127 antigens (from Biolegend). The cocktail of antibodies staining for lineage antigens for L-GMPs consisted of Biotin labelled anti-mouse antibodies against, CD3e, CD4, CD8, CD19, B220, Gr-1 (from BD Biosciences) and CD127 antigens (from Biolegend). Flow sorting was performed using the BD FACSAria II. Analysis was performed using FlowJo Software (TreeStar Software). Secondary stains were performed as previously described.

Cell viability was analyzed using Annexin V-APC (BioLegend) and 7-AAD (ThermoFisher Scientific) with Annexin V⁻7-AAD⁻ cells considered as viable cells. Analysis was performed on A BD LSR II flow cytometer.

### Methylcellulose Assays

Ten thousand N-GMPs or L-GMPs infected with lentiviruses expressing relevant shRNAs were added to 3 cc of methylcellulose (StemCell Technologies) plated per well of a 6 well plate. Number of cells were counted on day 7.

### Metzeler Database Analysis

Raw CEL file data was downloaded from the Metzeler database from GEO using the Bioconductor package, GEOquery and processed using the ‘affy’ BioConductor package. Arrays were quality-controlled with arrayQualityMetrics and RMA normalized. Median probe intensities for genes of interest were calculated and combined with survival data and subjected to survival analysis.

### Estimation of Oxidative Pentose Phosphate Pathway Activity

Thp1 cells transduced with lentivirus expressing Aldh3a2 shRNA B or Control shRNA were cultured in glucose-free Roswell Park Memorial Institute medium (RPMI, ThermoFisher Scientific) supplemented with 10 mM 1,2-¹³C2-glucose (Cambridge Isotope Laboratories) for 24 hours. Polar metabolites were isolated through methanol-chloroform extraction and derivatized by a two-step process. First, 15 μl of methoxyamine in pyridine (MOX Reagent, Thermo Pierce) was added before incubation at 40° C. for 1.5 h. Next, 20 μl N-(tert-butyldimethylsilyl)-N-methyl-trifluoroacetamide, with 1% tert-Butyldimethylchlorosilane (TBDMS) (Sigma) was added, and samples were incubated at 60° C. for 1 h. The reaction mixtures were quickly vortexed, centrifuged at 21,000×g for 1 min, and supernatant was transferred to GC-MS vials for analysis.

Polar metabolites were analyzed on a 6890N GC with a DB-35 ms Ultra Inert capillary column coupled to a 5975B Inert XL MS (Agilent). The flow rate of the helium carrier gas (Airgas) was maintained at 1 ml/min. The inlet temperature was held at 270° C. Injection volumes and split ratios ranged from 2 μl splitless to 1 μl with a 1:10 split, depending on sample concentration and detected ion abundances. Both scan and selected ion monitoring (SIM) modes were used to detect measured ions (with SIM parameters identical to previously published values. The instrument was operated in electron ionization mode with an energy of 70 eV. For polar metabolite samples, the GC oven was first held at 100° C. for 3 min, then ramped at 2.5° C./min to 300° C.; masses were profiled from 150 to 625 amu when in scan mode. Raw abundance data was converted to mass isotopomer distributions (MIDs) and corrected for natural abundance using an in-house software operating in Matlab (MathWorks).

Activity of the oxidative pentose phosphate pathway was estimated by making the ratio of M+1 lactate over M+2 lactate as detailed in FIG. 12F.

### Fatty Acid Analysis

Long chain fatty acids were quantified in 100000 L-GMPs infected with control shRNA or shRNA 1 as previously described.

### Fatty Acid Rescue Experiments

Bulk GFP+ cells sorted from wildtype or mutant mice were plated in a 24 well plate at 50,000 cells per well. Oleic acid (Sigma), complexed to fatty acid-free bovine serum albumin, was added at concentrations of 62.5, 125 or 250 μM. Ethanol/albumin was used as control. 72 hours later cell viability was analyzed by flow cytometry.

### REFERENCES

1. Vander Heiden M G, Cantley L C, Thompson C B. Understanding the Warburg effect: the metabolic requirements of cell proliferation. Science 2009; 324(5930): 1029-1033. doi: 10.1126/science.1160809
2. Sancho P, Barneda D, Heeschen C. Hallmarks of cancer stem cell metabolism. British journal of cancer 2016; 114(12): 1305-1312. e-pub ahead of print 2016/05/25; doi: 10.1038/bjc.2016.152
3. Grubovikj R M, Alavi A, Koppel A, Territo M, Schiller G J. Minimal residual disease as a predictive factor for relapse after allogeneic hematopoietic stem cell transplant in adult patients with acute myeloid leukemia in first and second complete remission. Cancers (Basel) 2012; 4(2): 601-617. e-pub ahead of print 2012/01/01; doi: 10.3390/cancers4020601
4. Ho T C, LaMere M, Stevens B M, Ashton J M, Myers J R, O'Dwyer K M et al. Evolution of acute myelogenous leukemia stem cell properties after treatment and progression. Blood 2016; 128(13): 1671-1678. e-pub ahead of print 2016/07/17; doi: 10.1182/blood-2016-02-695312
5. Eppert K, Takenaka K, Lechman E R, Waldron L, Nilsson B, van Galen P et al. Stem cell gene expression programs influence clinical outcome in human leukemia. Nature medicine 2011; 17(9): 1086-1093. e-pub ahead of print 2011/08/30; doi: 10.1038/nm.2415
6. Gentles A J, Plevritis S K, Majeti R, Alizadeh A A. Association of a leukemic stem cell gene expression signature with clinical outcomes in acute myeloid leukemia. JAMA 2010; 304(24): 2706-2715. e-pub ahead of print 2010/12/24; doi: 10.1001/jama.2010.1862
7. Pollyea D A, Jordan C T. Therapeutic targeting of acute myeloid leukemia stem cells. Blood 2017; 129(12): 1627-1635. e-pub ahead of print 2017/02/06; doi: 10.1182/blood-2016-10-696039
8. Jones C L, Stevens B M, D'Alessandro A, Reisz J A, Culp-Hill R, Nemkov T et al. Inhibition of Amino Acid Metabolism Selectively Targets Human Leukemia Stem Cells. Cancer cell 2018; 34(5): 724-740 e724. e-pub ahead of print 2018/11/14; doi: 10.1016/j.ccell.2018.10.005
9. Wang Y H, Israelsen W J, Lee D, Yu V W, Jeanson N T, Clish C B et al. Cell-state-specific metabolic dependency in hematopoiesis and leukemogenesis. Cell 2014; 158(6): 1309-1323. doi: 10.1016/j.cell.2014.07.048
10. Pei S, Minhajuddin M, Adane B, Khan N, Stevens B M, Mack S C et al. AMPK/FIS1-Mediated Mitophagy Is Required for Self-Renewal of Human AML Stem Cells. Cell stem cell 2018; 23(1): 86-100 e106. e-pub ahead of print 2018/06/19; doi: 10.1016/j.stem.2018.05.021
11. Kuntz E M, Baquero P, Michie A M, Dunn K, Tardito S, Holyoake T L et al. Targeting mitochondrial oxidative phosphorylation eradicates therapy-resistant chronic myeloid leukemia stem cells. Nature medicine 2017; 23(10): 1234-1240. e-pub ahead of print 2017/09/19; doi: 10.1038/nm.4399
12. Lagadinou E D, Sach A, Callahan K, Rossi R M, Neering S J, Minhajuddin M et al. BCL-2 inhibition targets oxidative phosphorylation and selectively eradicates quiescent human leukemia stem cells. Cell stem cell 2013; 12(3): 329-341. e-pub ahead of print 2013/01/22; doi: 10.1016/j.stem.2012.12.013
13. Sykes D B, Kfoury Y S, Mercier F E, Wawer M J, Law J M, Haynes M K et al. Inhibition of Dihydroorotate Dehydrogenase Overcomes Differentiation Blockade in Acute Myeloid Leukemia. Cell 2016; 167(1): 171-186 e115. e-pub ahead of print 2016/09/20; doi: 10.1016/j.cell.2016.08.057
14. Adane B, Ye H, Khan N, Pei S, Minhajuddin M, Stevens B M et al. The Hematopoietic Oxidase NOX2 Regulates Self-Renewal of Leukemic Stem Cells. Cell reports 2019; 27(1): 238-254 e236. e-pub ahead of print 2019/04/04; doi: 10.1016/j.celrep.2019.03.009
15. Vangapandu H V, Ayres M L, Bristow C A, Wierda W G, Keating M J, Balakrishnan K et al. The Stromal Microenvironment Modulates Mitochondrial Oxidative Phosphorylation in Chronic Lymphocytic Leukemia Cells. Neoplasia 2017; 19(10): 762-771. e-pub ahead of print 2017/09/02; doi: 10.1016/j.neo.2017.07.004
16. Hangauer M J, Viswanathan V S, Ryan M J, Bole D, Eaton J K, Matov A et al. Drug-tolerant persister cancer cells are vulnerable to GPX4 inhibition. Nature 2017; 551(7679): 247-250. e-pub ahead of print 2017/11/02; doi: 10.1038/nature24297
17. Viswanathan V S, Ryan M J, Dhruv H D, Gill S, Eichhoff O M, Seashore-Ludlow B et al. Dependency of a therapy-resistant state of cancer cells on a lipid peroxidase pathway. Nature 2017; 547(7664): 453-457. e-pub ahead of print 2017/07/06; doi: 10.1038/nature23007
18. Kastrinaki M C, Andreakou I, Charbord P, Papadaki H A. Isolation of human bone marrow mesenchymal stem cells using different membrane markers: comparison of colony/cloning efficiency, differentiation potential, and molecular profile. Tissue Eng Part C Methods 2008; 14(4): 333-339. e-pub ahead of print 2008/09/20; doi: 10.1089/ten.tec.2008.0173
19. Kamentsky L, Jones T R, Fraser A, Bray M A, Logan D J, Madden K L et al. Improved structure, function and compatibility for CellProfiler: modular high-throughput image analysis software. Bioinformatics 2011; 27(8): 1179-1180. e-pub ahead of print 2011/02/26; doi: 10.1093/bioinformatics/btr095
20. Saez B, Ferraro F, Yusuf R Z, Cook C M, Yu V W, Pardo-Saganta A et al. Inhibiting stromal cell heparan sulfate synthesis improves stem cell mobilization and enables engraftment without cytotoxic conditioning. Blood 2014; 124(19): 2937-2947. e-pub ahead of print 2014/09/10; doi: 10.1182/blood-2014-08-593426
21. Kelson T L, Secor McVoy J R, Rizzo W B. Human liver fatty aldehyde dehydrogenase: microsomal localization, purification, and biochemical characterization. Biochimica et biophysica acta 1997; 1335(1-2): 99-110. e-pub ahead of print 1997/04/17;
22. Rizzo W B, Craft D A. Sjogren-Larsson syndrome. Deficient activity of the fatty aldehyde dehydrogenase component of fatty alcohol:NAD+ oxidoreductase in cultured fibroblasts. The Journal of clinical investigation 1991; 88(5): 1643-1648. e-pub ahead of print 1991/11/01; doi: 10.1172/JC1115478
23. Krivtsov A V, Twomey D, Feng Z, Stubbs M C, Wang Y, Faber J et al. Transformation from committed progenitor to leukaemia stem cell initiated by MLL-AF9. Nature 2006; 442(7104): 818-822. e-pub ahead of print 2006/07/25; doi: 10.1038/nature04980
24. Nelson D L, Cox M M. Lehninger Principles of Biochemistry, 5th Edition edn W H Freeman and Co, 2008.
25. Moffat J, Grueneberg D A, Yang X, Kim S Y, Kloepfer A M, Hinkle G et al. A lentiviral RNAi library for human and mouse genes applied to an arrayed viral high-content screen. Cell 2006; 124(6): 1283-1298. e-pub ahead of print 2006/03/28; doi: 10.1016/j.cell.2006.01.040
26. German N J, Yoon H, Yusuf R Z, Murphy J P, Finley L W, Laurent G et al. PHD3 Loss in Cancer Enables Metabolic Reliance on Fatty Acid Oxidation via Deactivation of ACC2. Molecular cell 2016; 63(6): 1006-1020. e-pub ahead of print 2016/09/17; doi: 10.1016/j.molcel.2016.08.014
27. Ito K, Carracedo A, Weiss D, Arai F, Ala U, Avigan D E et al. A PML-PPAR-delta pathway for fatty acid oxidation regulates hematopoietic stem cell maintenance. Nature medicine 2012; 18(9): 1350-1358. e-pub ahead of print 2012/08/21; doi: 10.1038/nm.2882
28. Muramoto G G, Russell J L, Safi R, Salter A B, Himburg H A, Daher P et al. Inhibition of aldehyde dehydrogenase expands hematopoietic stem cells with radioprotective capacity. Stem Cells 2010; 28(3): 523-534. e-pub ahead of print 2010/01/08; doi: 10.1002/stem.299
29. Gasparetto M, Smith C A. ALDHs in normal and malignant hematopoietic cells: Potential new avenues for treatment of AML and other blood cancers. Chemico-biological interactions 2017; 276: 46-51. e-pub ahead of print 2017/06/25; doi: 10.1016/j.cbi.2017.06.020
30. Metzeler K H, Hummel M, Bloomfield C D, Spiekermann K, Braess J, Sauerland M C et al. An 86-probe-set gene-expression signature predicts survival in cytogenetically normal acute myeloid leukemia. Blood 2008; 112(10): 4193-4201. e-pub ahead of print 2008/08/22; doi: 10.1182/blood-2008-02-134411
31. Dixon S J, Lemberg K M, Lamprecht M R, Skouta R, Zaitsev E M, Gleason C E et al. Ferroptosis: an iron-dependent form of nonapoptotic cell death. Cell 2012; 149(5): 1060-1072. e-pub ahead of print 2012/05/29; doi: 10.1016/j.cell.2012.03.042
32. Jones C L, Stevens B M, D'Alessandro A, Reisz J A, Culp-Hill R, Nemkov T et al. Inhibition of Amino Acid Metabolism Selectively Targets Human Leukemia Stem Cells. Cancer cell 2019; 35(2): 333-335. e-pub ahead of print 2019/02/13; doi: 10.1016/j.ccell.2019.01.013
33. Yu Y, Xie Y, Cao L, Yang L, Yang M, Lotze M T et al. The ferroptosis inducer erastin enhances sensitivity of acute myeloid leukemia cells to chemotherapeutic agents. Mol Cell Oncol 2015; 2(4): e1054549. e-pub ahead of print 2016/06/17; doi: 10.1080/23723556.2015.1054549
34. Rizzo W B, Craft D A. Sjogren-Larsson syndrome: accumulation of free fatty alcohols in cultured fibroblasts and plasma. Journal of lipid research 2000; 41(7): 1077-1081. e-pub ahead of print 2000/07/07;
35. Singh S, Brocker C, Koppaka V, Chen Y, Jackson B C, Matsumoto A, Thompson D C, Vasiliou V., Aldehyde dehydrogenases in cellular responses to oxidative/electrophilic stress. Free Radic Biol Med. 2013 March; 56:89-101. doi: 10.1016/j.freeradbiomed.2012.11.010. Epub 2012 Nov. 27.
36. Yang W S, Kim K J, Gaschler M M, Patel M, Shchepinov M S, Stockwell B R., Peroxidation of polyunsaturated fatty acids by lipoxygenases drives ferroptosis. Proc Natl Acad Sci USA. 2016 Aug. 23; 113(34):E4966-75. doi: 10.1073/pnas.1603244113. Epub 2016 Aug. 9.
37. Magtanong L, Ko P J, To M, Cao J Y, Forcina G C, Tarangelo A, Ward C C, Cho K, Patti G J, Nomura D K, Olzmann J A, Dixon S J., Exogenous Monounsaturated Fatty Acids Promote a Ferroptosis-Resistant Cell State. Cell Chem Biol. 2019 Mar. 21; 26(3):420-432.e9. doi: 10.1016/j.chembiol.2018.11.016. Epub 2019 Jan. 24.
38. Akashi K, Traver D, Miyamoto T, Weissman I L. A clonogenic common myeloid progenitor that gives rise to all myeloid lineages. Nature 2000; 404(6774): 193-197. e-pub ahead of print 2000/03/21; doi: 10.1038/35004599
39. Davis S, Meltzer P S. GEOquery: a bridge between the Gene Expression Omnibus (GEO) and BioConductor. Bioinformatics 2007; 23(14): 1846-1847. e-pub ahead of print 2007/05/15; doi: 10.1093/bioinformatics/btm254
40. Gautier L, Cope L, Bolstad B M, Irizarry R A. affy—analysis of Affymetrix GeneChip data at the probe level. Bioinformatics 2004; 20(3): 307-315. e-pub ahead of print 2004/02/13; doi: 10.1093/bioinformatics/btg405
41. Kauffmann A, Gentleman R, Huber W. arrayQualityMetrics—a bioconductor package for quality assessment of microarray data. Bioinformatics 2009; 25(3): 415-416. e-pub ahead of print 2008/12/25; doi: 10.1093/bioinformatics/btn647
42. Irizarry R A, Bolstad B M, Collin F, Cope L M, Hobbs B, Speed T P. Summaries of Affymetrix GeneChip probe level data. Nucleic acids research 2003; 31(4): e15. e-pub ahead of print 2003/02/13;
43. Ahn W S, Antoniewicz M R. Metabolic flux analysis of CHO cells at growth and non-growth phases using isotopic tracers and mass spectrometry. Metabolic engineering 2011; 13(5): 598-609. e-pub ahead of print 2011/08/09; doi: 10.1016/j.ymben.2011.07.002
44. Fernandez C A, Des Rosiers C, Previs S F, David F, Brunengraber H. Correction of 13C mass isotopomer distributions for natural stable isotope abundance. J Mass Spectrom 1996; 31(3): 255-262. e-pub ahead of print 1996/03/01; doi: 10.1002/(SICI)1096-9888(199603)31:3<255::AID-JMS290>3.0.CO;2-3
45. Rizzo W B, Watkins P A, Phillips M W, Cranin D, Campbell B, Avigan J. Adrenoleukodystrophy: oleic acid lowers fibroblast saturated C22-26 fatty acids. Neurology 1986; 36(3): 357-361. e-pub ahead of print 1986/03/01.

1. A method of inducing cell death of cancer cells in a population of cells comprising contacting the population of cells with an effective amount of a first agent that inhibits Aldh3a2 activity or expression and an effective amount of a second agent that induces ferroptosis.

2. The method of claim 1, wherein the method also inhibits the growth of the cancer cells.

3. The method of claim 1, wherein the method does not induce or does not substantially induce cell death of non-cancer cells.

4. The method of claim 1, wherein the uncontacted cancer cells exhibit increased redox stress or excessive production of reactive oxygen species.

5. The method of claim 1, wherein the cancer cells are leukemia cells.

6. The method of claim 5, wherein the cancer cells are acute myeloid leukemia (AML) cells.

7. (canceled)

8. The method of claim 1, wherein the first agent is a short hairpin RNA (shRNA) inhibiting Aldh3a2 expression.

9. (canceled)

10. The method of claim 1, wherein the second agent is a glutathione peroxidase 4 (GPX4) inhibitor.

11. The method of claim 10, wherein the GPX4 inhibitor is erastin, RSL3, ML162, or DPI10.

12. (canceled)

13. The method of claim 1, wherein the cancer cells are human cancer cells.

14. (canceled)

15. An anti-cancer composition comprising a first agent which inhibits Aldh3a2 activity or expression, and a second agent which induces ferroptosis.

16. (canceled)

17. The composition of claim 15, wherein the first agent is a short hairpin RNA (shRNA) inhibiting Aldh3a2 expression.

18. (canceled)

19. The composition of claim 15, wherein the second agent is a glutathione peroxidase 4 (GPX4) inhibitor.

20. The composition of claim 19, wherein the GPX4 inhibitor is erastin, RSL3, ML162, or DPI10.

21. The composition of claim 15, wherein the composition is formulated for administration by a mode selected from the group consisting of: topically, by injection, by intravenous injection, by inhalation, continuous release by depot or pump, and a combination thereof.

22. A method of treating cancer in a subject in need thereof, comprising administering to the subject an effective amount of a first agent that inhibits Aldh3a2 activity or expression and a chemotherapeutic regimen.

23. The method of claim 22, wherein the chemotherapeutic regimen is an induction chemotherapy treatment regimen.

24. The method of claim 23, wherein the induction chemotherapy regimen comprises administering an antimetabolite agent and an anthracycline agent to the subject.

25. The method of claim 22, wherein the cancer is leukemia.

26. The method of claim 22, wherein the subject is human.


SEQUENCE LISTING (2 sequences)

SEQ ID NO: 1 - DNA, 21 bp, Artificial Sequence
  shRNA DNA vector sequence
gcaggaaatgctgtgattata

SEQ ID NO: 2 - DNA, 21 bp, Artificial Sequence
  shRNA DNA vector sequence
cctctggctctttatgtattt